(19) United States
(12) Patent Application Publication
(10) Pub. No.: US 2023/0383249 A1
(43) Pub. Date: Nov. 30, 2023

(54) ALLOGENEIC THERAPEUTIC CELLS

(71) Applicant: Kite Pharma, Inc., Foster City, CA (US)

(72) Inventors: Jun Feng, San Diego, CA (US); Chloe Knights Krishnamurty, Berkeley, CA (US); Kimberly Sue Schluns, Oakland, CA (US)

(21) Appl. No.: 18/175,049

(22) Filed: Feb. 27, 2023

Related U.S. Application Data
(60) Provisional application No. 63/314,848, filed on Feb. 28, 2022.

(51) Int. Cl. C12N 5/078; A61P 35/00; C12N 9/22; C07K 14/725

(52) U.S. Cl. CPC C12N 5/0634; A61P 35/00; C12N 9/22; C07K 14/7051; C12N 2510/00; C12N 2310/20

(57) ABSTRACT

The present disclosure provides allogeneic cells that cause reduced, minimal, or no risks of graft-versus-host disease (GVHD) and reduced, minimal, or no risk of CD4, CD8 and NK cell rejections, for example, when used to treat diseases in a patient. The allogeneic cells may be genetically engineered to reduce the expression or activity of MHC class I and/or MHC class II molecules, such as knocking out the RFX5 gene. Methods of preparing and using such allogeneic cells are also provided.

### CROSS-REFRENCE TO RELATED APPLICATIONS

The present application claims the priority benefit of U.S. Provisional Application No. 63/314,848, filed Feb. 28, 2022, which is hereby incorporated by reference in its entirety.

### SEQUENCE LISTING

The instant application contains a Sequence Listing which has been submitted electronically in XML file format and is hereby incorporated by reference in its entirety. Said XML copy, created on Feb. 22, 2023, is named K-1138-US-NP_SL.xml and is 147,558 bytes in size.

### TECHNICAL FIELD

The present disclosure relates to the field of cell therapy, and more specifically, genetically engineered immune cells.

### BACKGROUND

T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. Chimeric antigen receptors (CARs) and engineered T cell receptors (TCRs), which comprise binding domains capable of interacting with a specific tumor antigen, allow T cells to target and kill cancer cells that express the specific tumor antigen.

CD19-directed chimeric antigen receptor T cells (CAR T cells) have demonstrated potent anti-tumor efficacy in treating a range of B-cell malignancies. However, the autologous CAR T therapy presents technical, manufacturing, and commercial constraints, which may limit its clinical application to the full potential. Allogeneic CAR T therapy is an alternative strategy to overcome the inherent limitations of autologous therapy and provide an “off-the-shelf” approach for clinical use.

In principle, an allogeneic CAR T therapy employs T cells from healthy human donors that subsequently undergo gene modifications to confer specificity against tumor antigens. Additionally, gene edits are also introduced to prevent graft-versus-host disease (GVHD) and the rejection of allogeneic CAR T cells by the patient's immune system. GVHD is mainly attributed to the interactions between the T-cell receptor (TCR)αβ protein on donor T cells and the mismatched human leukocyte antigen (HLA) molecules on recipient patient cells. Inversely, the host's endogenous CD8⁺ T cells can interact and eliminate donor T-cell grafts bearing mismatched major histocompatibility complex (MHC) class I molecules.

### SUMMARY

Efforts have been made to develop hypoimmunogenic cells, in particular immune cells such as T and NK cells, suitable for off-shelf use. In one example, beta-2-microglobulin (B2M), a major component of MHC class I molecules, is inactivated in an allogeneic T cell. Inactivation of B2M can eliminate MHC class I, which is believed to reduce or prevent rejection by mismatched CD8 T cells in the host. It was observed that, however, B2M knockout CAR T cells are susceptible to killing by the host's NK cells, as NK cells become stimulated and kill allogenic T cells that lacked MHC class I activity.

The present disclosure, in various embodiments, provides cell engineering approaches that are superior to B2M knockout. The approaches can achieve minimal or no risk of GVHD and minimal or no risk of CD4, CD8 and NK cell rejections, while retaining comparable or even improved therapeutic activities.

In accordance with one embodiment of the present disclosure, therefore, provided is an allogeneic cell that is genetically engineered to reduce the expression or activity of MHC class I or MHC class II. In some embodiments, the MHC class I expression or activity is reduced but not eliminated. In some embodiments, the MHC class II expression and/or activity may be reduced or even eliminated. Also provided are methods for preparing such cells.

One embodiment of the present disclosure provides an isolated human immune cell engineered to have MHC class I activity or expression that is from 10% to 80% lower as compared to a corresponding non-engineered immune cell. In some embodiments, the cell has MHC class II activity or expression that is at least 20% lower, or at least 40% lower, as compared a corresponding non-engineered immune cell. In some embodiments, the cell has MHC class II activity or expression that is at least 75% lower as compared a corresponding non-engineered immune cell or reference cell.

In some embodiments, the corresponding or reference immune cell is an immune cell that has not been engineered to have reduced expression of MHC class I and/or MHC class II molecules. In some embodiments, the corresponding or reference immune cell is an immune cell that has been engineered to have reduced expression of MHC class I, but not reduced expression of MHC class II.

In some embodiments, the cell is a T cell or a NK cell, or any other immune cell such as monocyte or macrophage, or a cell derived from a stem cell such as iPSC. In some embodiments, the cell comprises an exogenous polynucleotide encoding a chimeric antigen receptor (CAR) or a T-cell receptor (TCR). In some embodiments, the CAR recognizes CD19 and/or CD20. In some embodiments, the CAR comprises the amino acid sequence of SEQ ID NO:26 or 27 or a sequence having at least about 90% sequence identity to SEQ ID NOS: 26 or 27.

In some embodiments, the CAR recognizes CLL-1. In some embodiments, the CAR comprises the amino acid sequence of any of SEQ ID NOS:65-76 or 78-83 or a sequence having at least about 90% sequence identity to any of SEQ ID NOS:65-76 or 78-83.

In some embodiments, the endogenous gene of at least one of RFX5 (regulatory factor X5), TAP1 (Transporter associated with antigen processing 1), TAP2 (Antigen peptide transporter 2), or CIITA (class II, major histocompatibility complex, transactivator) is inactivated or reduced. In some embodiments, both alleles of the endogenous gene are inactivated. In some embodiments, RFX5 is inactivated in the cell. In some embodiments, the endogenous genes of TAP1, TAP2, and CIITA are not engineered.

In some embodiments, the endogenous gene of TRAC (T Cell Receptor Alpha Constant) is further inactivated or reduced. In some embodiments, the endogenous gene of B2M (Beta-2-microglobulin) is not engineered, or wherein the cell has normal activity of B2M.

In some embodiments, the inactivation is achieved with (a) editing of the endogenous gene, (b) expression of an inhibitory RNA, or (c) an inhibitor, preferably an antibody. In some embodiments, the editing is by CRISPR/Cas9, a zinc finger nuclease (ZFN), a TALEN, a MegaTAL, a meganuclease, Cpf1, homologous recombination, a single stranded oligodeoxynucleotide (ssODN), or base editing.

Methods for the cells are also provided, without limitation. In some embodiments, the CAR or TCR is introduced to the cell prior to editing of the gene such as RFX5. Also provided are methods of using the cells for treating a disease.

### DETAILED DESCRIPTION OF THE DISCLOSURE

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Administering” refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “activated” and “activation” refer to the state of a T cell that has been sufficiently stimulated to induce detectable cellular proliferation. In one embodiment, activation may also be associated with induced cytokine production, and detectable effector functions. The term “activated T cells” refers to, among other things, T cells that are proliferating. Signals generated through the TCR alone may be insufficient for full activation of the T cell and one or more secondary or costimulatory signals may also be required. Thus, T cell activation comprises a primary stimulation signal through the TCR/CD3 complex and one or more secondary costimulatory signals. Costimulation may be evidenced by proliferation and/or cytokine production by T cells that have received a primary activation signal, such as stimulation through the TCR/CD3 complex.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecule. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR). In certain embodiments, the antigen binding molecule binds to 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), β2-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DR5, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor α, Folate receptor β, Fos-related antigen 1, Fucosyl, Fucosyl GM1; GM2, ganglioside G2 (GD2), ganglioside GD3 (aNeu5Ac(2-8)aNeu5Ac(2-3)bDGalp(1-4)bDG1cp(1-1)Cer), o-acetyl-GD2 ganglioside (OAcGD2), GITR (TNFRSF 18), GM1, ganglioside GM3 (aNeu5Ac(2-3)bDGalp(1-4)bDG1cp(1-1)Cer), GP 100, hexasaccharide portion of globoH glycoceramide (GloboH), glycoprotein 75, Glypican-3 (GPC3), glycoprotein 100 (gplOO), GPNMB, G protein-coupled receptor 20 (GPR20), G protein-coupled receptor class C group 5, member D (GPRCSD), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgGl, immunoglobulin lambda-like polypeptide 1 (IGLL1), IL-6, Interleukin 11 receptor alpha (IL-llRa), IL-13, Interleukin-13 receptor subunit alpha-2 (IL-13Ra2 or CD213A2), insulin-like growth factor I receptor (IGF1-R), integrin α5β1, integrin αvβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-la, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis(Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTl, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUC1), MUC2, MUC3, MUC4, MUCSAC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA, NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PR1), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHE), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1). Amino acid sequences that specifically bind to said antigens are known in the art or may be prepared using methods known in the art; examples include immunoglobulins, variable regions of immunoglobulins (e.g., variable fragment (“Fv”) or bivalent variable fragment (“Fab”)), single chain antibodies, etc. In certain embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

An “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of a TCR or CAR contemplated herein, is designed to bind. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens. In one particular embodiment, the antigen is all or a fragment of 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), β2-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DR5, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor α, Folate receptor β, Fos-related antigen 1, Fucosyl, Fucosyl GM1; GM2, ganglioside G2 (GD2), ganglioside GD3 (aNeu5Ac(2-8)aNeu5Ac(2-3)bDGalp(1-4)bDG1cp(1-1)Cer), o-acetyl-GD2 ganglioside (OAcGD2), GITR (TNFRSF 18), GM1, ganglioside GM3 (aNeu5Ac(2-3)bDGalp(1-4)bDG1cp(1-1)Cer), GP 100, hexasaccharide portion of globoH glycoceramide (GloboH), glycoprotein 75, Glypican-3 (GPC3), glycoprotein 100 (gp100), GPNMB, G protein-coupled receptor 20 (GPR20), G protein-coupled receptor class C group 5, member D (GPRCSD), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgGl, immunoglobulin lambda-like polypeptide 1 (IGLL1), IL-6, Interleukin 11 receptor alpha (IL-l lRa), IL-13, Interleukin-13 receptor subunit alpha-2 (IL-13Ra2 or CD213A2), insulin-like growth factor I receptor (IGF1-R), integrin α5β1, integrin αvβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-la, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis(Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTl, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUC1), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA, NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PR1), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog

(SHE), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1). A “target” is any molecule bound by a binding motif, antigen binding system, CAR or antigen binding agent, e.g., an antibody.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

The term “extracellular ligand-binding domain,” as used herein, refers to an oligo- or polypeptide that is capable of binding a ligand, e.g., a cell surface molecule. For example, the extracellular ligand-binding domain may be chosen to recognize a ligand that acts as a cell surface marker on target cells associated with a particular disease state (e.g., cancer). Examples of cell surface markers that may act as ligands include those associated with viral, bacterial and parasitic infections, autoimmune disease and cancer cells.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered.

The “transmembrane” region or domain is the portion of the CAR that anchors the extracellular binding portion to the plasma membrane of the immune effector cell, and facilitates binding of the binding domain to the target antigen. The transmembrane domain may be a CD3 zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In one embodiment, the transmembrane domain is the transmembrane domain of CD137. In certain embodiments, the transmembrane domain is synthetic in which case it would comprise predominantly hydrophobic residues such as leucine and valine.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22, CD79a, CD79b and CD66d.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1 BB (CD137), 0X40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD3zeta and 4-1 BB, other costimulatory domains are contemplated for use with the CARs described herein. The inclusion of one or more co stimulatory signaling domains may enhance the efficacy and expansion of T cells expressing CAR receptors. The intracellular signaling and costimulatory signaling domains may be linked in any order in tandem to the carboxyl terminus of the transmembrane domain.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

A “costimulatory signal” refers to a signal, which in combination with a primary signal, such as TCR/CD3 ligation, leads to a T cell response, such as, but not limited to, proliferation and/or upregulation or down regulation of key molecules.

A “costimulatory ligand” includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS- L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDl la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

A “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof can be replaced with an amino acid residue with a similar side chain. In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution. Exemplary amino acid categorizations are summarized in Table 1 below:

TABLE 1
Amino acid categorization

| | | | | Hydropathy |
|---|---|---|---|---|
| Amino Acid | 3-Letter | Property | Property | Index |
| Alanine | Ala | nonpolar | neutral | 1.8 |
| Arginine | Arg | polar | positive | −4.5 |
| Asparagine | Asn | polar | neutral | −3.5 |
| Aspartic acid | Asp | polar | negative | −3.5 |
| Cysteine | Cys | nonpolar | neutral | 2.5 |
| Glutamic acid | Glu | polar | negative | −3.5 |
| Glutamine | Gln | polar | neutral | −3.5 |
| Glycine | Gly | nonpolar | neutral | −0.4 |
| Histidine | His | polar | positive | −3.2 |
| Isoleucine | Ile | nonpolar | neutral | 4.5 |
| Leucine | Leu | nonpolar | neutral | 3.8 |
| Lysine | Lys | polar | positive | −3.9 |
| Methionine | Met | nonpolar | neutral | 1.9 |
| Phenylalanine | Phe | nonpolar | neutral | 2.8 |
| Proline | Pro | nonpolar | neutral | −1.6 |
| Serine | Ser | polar | neutral | −0.8 |
| Threonine | Thr | polar | neutral | −0.7 |
| Tryptophan | Trp | nonpolar | neutral | −0.9 |
| Tyrosine | Tyr | polar | neutral | −1.3 |
| Valine | Val | nonpolar | neutral | 4.2 |

A “T cell receptor” or “TCR” refers to antigen-recognition molecules present on the surface of T cells. During normal T cell development, each of the four TCR genes, α, β, γ, and δ, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

A “patient” includes any human who is afflicted with a cancer (e.g., a lymphoma or a leukemia). The terms “subject” and “patient” are used interchangeably herein.

The terms “subject” and “patient” include human and non-human animal subjects as well as those with formally diagnosed disorders, those without formally recognized disorders, those receiving medical attention, and those at risk of developing the disorders.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

“Regulatory T cells” (“Treg”, “Treg cells”, or “Tregs”) refer to a lineage of CD4+ T lymphocytes that participate in controlling certain immune activities, e.g., autoimmunity, allergy, and response to infection. Regulatory T cells may regulate the activities of T cell populations, and may also influence certain innate immune system cell types. Tregs may be identified by the expression of the biomarkers CD4, CD25 and Foxp3, and low expression of CD127. Naturally occurring Treg cells normally constitute about 5-10% of the peripheral CD4+ T lymphocytes. However, Treg cells within a tumor microenvironment (i.e., tumor-infiltrating Treg cells), may make up as much as 20-30% of the total CD4+ T lymphocyte population.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. Thus, each zinc finger of a multi-finger ZFP includes a recognition helix region for binding to DNA within a backbone. The term zinc finger DNA binding protein is often abbreviated as zinc finger protein or ZFP. The term “zinc finger nuclease” includes one ZFN as well as a pair of ZFNs (the members of the pair are referred to as “left and right” or “first and second” or “pair”) that dimerize to cleave the target gene.

A “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205. Zinc finger and TALE DNA-binding domains can be “engineered” to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also International Patent Publication Nos. WO 98/53058; WO 98/53059; WO 98/53060; WO 02/016536; and WO 03/016496. The term “TALEN” includes one TALEN as well as a pair of TALENs (the members of the pair are referred to as “left and right” or “first and second” or “pair”) that dimerize to cleave the target gene.

CRISPR/Cas (Clustered regularly interspaced short palindromic repeats/CRISPR-associated protein) system has been the most powerful genomic editing tool since its conception for its unparalleled editing efficiency, convenience, and the potential applications in living organism. Directed by guide RNA (gRNA), a Cas nuclease can generate DNA double strand breaks (DSBs) at the targeted genomic sites in various cells (both cell lines and cells from living organisms). These DSBs are then repaired by the endogenous DNA repair system, which could be utilized to perform desired genome editing.

Base editors (BE), which integrate the CRISPR/Cas system with the APOBEC (apolipoprotein B mRNA editing enzyme, catalytic polypeptide-like) cytosine deaminase family, were recently developed that greatly enhanced the efficiency of CRISPR/Cas9-mediated gene correction. Through fusion with Cas9 nickase (nCas9) or catalytically dead Cas9 (dCas9), the cytosine (C) deamination activity of rat APOBEC1 (rA1) can be purposely directed to the target bases in genome and to catalyze C to Thymine (T) substitutions at these bases.

Prime editing (PE) is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

### RFX5 Knockout Cells

Allogeneic donor cells from healthy donors have the potential to offer off-the-shelf cell products that can be applied on demand, at much lower costs as compared to autologous ones. With the advancement in gene editing technologies, attempts have been made to knock out or knock certain genes in order to develop hypoimmunogenic cells suitable for off-shelf use.

Beta-2-microglobulin (β2M or B2M) is a critical component of MHC class I molecules. Deletion of B2M can eliminate MHC class I, which has been demonstrated to reduce or prevent rejection by mismatched CD8 T cells in the host.

The instant inventors have developed an allogeneic anti-CD19 CAR T-cell product, in which portions of both the TCR alpha constant (TRAC) locus and B2M were deleted by zinc finger nucleases (ZFN), resulting in reduced expression of the proteins on the cell surface. It was observed that, however, these edited CAR T cells were susceptible to the host's NK cells, as NK cells became stimulated and killed the allogenic T cells that lacked MHC class I expression.

The instant inventors, therefore, looked for alternative gene editing approaches that are superior to B2M knockout and can achieve minimal or no risk of GVHD and minimal or no risk of CD4, CD8 and NK cell rejections, while retaining comparable or even improved therapeutic activities.

In accordance with one embodiment of the present disclosure, therefore, provided is an allogeneic cell that is genetically engineered to reduce the expression or activity of MHC class I or MHC class II. In some embodiments, the MHC class I expression or activity is reduced but not eliminated. In some embodiments, the MHC class II expression and/or activity may be reduced or even eliminated. Also provided are methods for preparing such cells.

In one embodiment, the MHC class I expression or activity is decreased for at least 10%, or at least 20%, 30%, 40%, 50%, 60%, 70%, 80% or 90% as compared to a reference allogeneic cell (e.g., a cell not so engineered, such as T cell with only TRAC knockout). In one embodiment, the MHC class I expression or activity is retained at a level that is at least 5%, or at least 10%, 15%, 20%, 30%, 40%, 50%, 60%, 70%, 80% or 90% as compared to the reference allogeneic cell. In one embodiment, the MHC class I expression or activity is about 5%-90%, 10%-80%, 20%-80%, 20%-70%, 30%-70%, 30%-60%, 40%-60%, 10%-60%, 10%-50%, 20%-60%, 20%-50%, 20%-40%, 10%-40%, or 10%-30%, as compared to the reference allogeneic cell.

In one embodiment, the MHC class II expression or activity is decreased for at least 10%, or at least 20%, 30%, 40%, 50%, 60%, 70%, 80% or 90% as compared to a reference allogeneic cell. In one embodiment, the MHC class II expression or activity is retained at a level that is at least 5%, or at least 10%, 15%, 20%, 30%, 40%, 50%, 60%, 70%, 80% or 90% as compared to the reference allogeneic cell. In one embodiment, the MHC class II expression or activity is about 5%-90%, 10%-80%, 20%-80%, 20%-70%, 30%-70%, 30%-60%, 40%-60%, 10%-60%, 10%-50%, 20%-60%, 20%-50%, 20%-40%, 10%-40%, or 10%-30%, as compared to the reference allogeneic cell.

In one embodiment, the expression or activity of both MHC class I and II is decreased for at least 10%, or at least 20%, 30%, 40%, 50%, 60%, 70%, 80% or 90% as compared to a reference allogeneic cell. In one embodiment, the expression or activity of both MHC class I and II is retained at a level that is at least 5%, or at least 10%, 15%, 20%, 30%, 40%, 50%, 60%, 70%, 80% or 90% as compared to the reference allogeneic cell.

In some embodiments, the reference immune cell has not been engineered to have reduced expression of MHC class I and/or MHC class II molecules. In some embodiments, the reference immune cell has been engineered to have reduced MHC class I expression, but not MHC class II expression (e.g., TRAC and B2M knockout). In some embodiments, the reference immune cell has been engineered by introducing an exogenous construct that expresses a CAR or TCR, but has not been engineered to have reduced expression of an MHC class I or MHC class II molecule. In some embodiments, the reference immune cell is a non-transduced (NTD) cell from a healthy donor that has not been engineered to have reduced expression of an MHC class I or MHC class II molecule.

In some embodiments, the expression or activity of MHC class I is reduced or knocked down while the expression of activity of MHC class II is knocked out or eliminated.

The term MHC class I also refers to human leukocyte antigen (HLA) class I. Examples of MHC class I molecules include, but are not limited to, B2M, individual HLA molecules (e.g., HLA-A, -B, -C, -E, -G), TAP1, TAP2, and/or genes associated with Bare Lymphocyte Syndrome I (BLSI).

The term MHC class II also refers to human leukocyte antigen (HLA) class II. Examples of MHC class II molecules include, but are not limited to transcription factors (e.g., RFXANK, RFXS, RFXAP, or RFXS) or transactivators (CHTA), genes associated with BLS II, and/or individual HLA molecules (e.g., HLA-DP, -DQ, -DR, -DiVI, -DO -alpha and beta chains).

Multiple candidate genes were investigated, including single knockouts of RFX5 (regulatory factor X5), TAP1 (Transporter associated with antigen processing 1), TAP2 (Antigen peptide transporter 2), and CIITA (class II, major histocompatibility complex, transactivator). It was discovered that RFX5 single knockout achieved optimal downregulation of MHC class I and elimination of MHC class II expression in the engineered cells. The MHC class I downregulation was sufficient to reduce rejection by mismatched CD8 T cells but did not promote killing by NK cells. The elimination of MHC class II expression also reduces responses by mismatched CD4 T cells. Also important, as demonstrated in the experimental examples, CAR cells with RFX5 knocked out had excellent manufacturability and CAR functionality.

Accordingly, one embodiment of the present disclosure provides a method for preparing an allogeneic cell with reduced activity in inducing graft-versus-host disease (GVHD) or host rejection. In some embodiment, the method entails reducing (or completely eliminating) the expression/activity of a gene involved in expression of MHC class I and/or II.

In some embodiments, the gene is selected from RFX5 (regulatory factor X5), TAP1 (Transporter associated with antigen processing 1), TAP2 (Antigen peptide transporter 2), or CIITA (class II, major histocompatibility complex, transactivator). In some embodiments, the gene is RFX5, and at least one of TAP1, TAP2 and CIITA is also inactivated or inhibited, completely or partially. In some embodiments, only the expression or activity of RFX5 is reduced in the cell, while no genetic change is made to TAP1, TAP2 and CIITA.

In some embodiments, the endogenous gene of B2M (Beta-2-microglobulin) in the cell is not engineered. That is, no gene editing is conducted to the B2M locus and no inhibitory agent is introduced to the cell.

In some embodiments, the cell is further engineered to reduce (or completely eliminate) the expression/activity of TRAC (T Cell Receptor Alpha Constant).

In some embodiments, the cell is derived from a healthy donor. In some embodiments, the cell is derived/differentiated from a stem cell, such as an induced pluripotent stem cell (iPSC).

In some embodiments, the cell is a T cell derived from a healthy donor.

### Techniques for Reducing the Expression/Activity of a Gene in A Cell

Methods of reducing or eliminating the expression or activity of a gene are known in the art. In some embodiments, such reduction or elimination includes any detectable decrease in the production of a gene (e.g., RFX5). In certain examples, detectable RFX5 in a cell decreases by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of RFX5 detected in a corresponding cell in which the RFX5 has not been inhibited).

In certain embodiments, detectable TCR in a cell decreases by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of TCR detected in a corresponding cell in which the TCR has not been inhibited).

In certain embodiments, detectable B2M in a cell decreases by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of B2M detected in a corresponding cell in which the B2M has not been inhibited).

In certain embodiments, reduction or elimination of gene expression occurs by direct inhibition of the gene (e.g., knocking down or knocking out the RFXS gene may reduce or eliminate expression or activity of RFX5). In other embodiments, reduction or elimination of gene expression occurs by indirect inhibition of the gene (e.g., knocking down or knocking out the RFX5 gene may reduce expression or activity of MHC class I molecules).

Percent decrease and percent increases can be calculated by methods known in the art. As a non-liming example, a percent reduction or decrease in expression or activity of a molecule in an edited cell (e.g., a cell comprising an RFX5 KO) relative to a reference or corresponding cell (e.g., a cell that does not comprise an RFX5 KO) may be calculated by subtracting the reference/corresponding cell value minus the edited cell value, dividing that amount the reference value, and then multiplying by 100 to get a percent decrease. If the percent is negative, that may mean that there was an increase and not a decrease.

In some embodiments, the expression or activity of a gene can be reduced with a suitable inhibiting agent, such as a small molecule inhibitor, an inhibitory RNA (e.g., siRNA, shRNA), or an antibody. In some embodiments, this can be achieved by genetic editing of the target gene, at one or both of the alleles of the target gene.

In certain embodiments, the expression of one or more of RFXS is reduced using a DNA-binding domain, for example coupled to a nuclease domain, that specifically binds to a target site in the RFX5 gene and mediates mutation at the target site thereby decreasing expression of functional RFX5. Any DNA-binding domain can be used in the compositions and methods disclosed herein, including but not limited to a zinc finger DNA-binding domain, a TALE DNA binding domain, the DNA-binding portion (sgRNA) of a CRISPR/Cas nuclease, or a DNA-binding domain from a meganuclease.

In certain embodiments, the DNA binding domain comprises a zinc finger protein. Preferably, the zinc finger protein is non-naturally occurring in that it is engineered to bind to a target site of choice. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence.

Usually, the ZFPs include at least three fingers. Certain of the ZFPs include four, five or six fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, which domains can be transcriptional activation or repression domains.

In some embodiments, the DNA-binding domain may be derived from a nuclease. For example, the recognition sequences of homing endonucleases and meganucleases such as I-Scel, I-CeuI, PI-PspI, PI-Sce, I-SceIV, I-CsmI, I-PanI, I-SceII, I-PpoI, I-SceIII, I-CreI, I-TevI, I-TevII and I-TevIII are known. In addition, the DNA-binding specificity of homing endonucleases and meganucleases can be engineered to bind non-natural target sites.

In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity.

In certain embodiments, the DNA-binding domain is part of a CRISPR/Cas nuclease system, including a single guide RNA (sgRNA) that binds to DNA. The CRISPR (clustered regularly interspaced short palindromic repeats) locus, which encodes RNA components of the system, and the cas (CRISPR-associated) locus, which encodes proteins make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

sgRNAs that may be suitable for use in the cells and methods of the present disclosure may be identified using CRISPR design tools. Exemplary sgRNA sequences are shown in Table 2.

TABLE 2
Exemplary guide RNAs

| Guide RNA | Sequence (5′ to 3′) |
|---|---|
| TRAC gRNA-1 | CTTCAAGAGCAACAGTGCTG (SEQ ID NO: 84) |
| TRAC gRNA-2 | CTCTCAGCTGGTACACGGCA (SEQ ID NO: 85) |
| TRAC gRNA-3 | TCTCTCAGCTGGTACACGGC (SEQ ID NO: 86) |
| TRAC gRNA-4 | GCTGGTACACGGCAGGGTCA (SEQ ID NO: 87) |
| B2M gRNA-1 | GGCCGAGATGTCTCGCTCCG (SEQ ID NO: 88) |
| B2M gRNA-2 | CGCGAGCACAGCTAAGGCCA (SEQ ID NO: 89) |
| B2M gRNA-3 | GAGTAGCGCGAGCACAGCTA (SEQ ID NO: 90) |
| B2M gRNA-4 | AAGTCAACTTCAATGTCGGA (SEQ ID NO: 91) |
| RFX5 gRNA-1 | TCGGAGCCTCTGAAGAAGGG (SEQ ID NO: 92) |
| RFX5 gRNA-2 | GCCAAGTACACTGAGCAATG (SEQ ID NO: 93) |
| RFX5 gRNA-3 | CAGGGGTGGCATAGACACCA (SEQ ID NO: 94) |
| RFX5 gRNA-4 | GCCAAGTACACTGAGCAATG (SEQ ID NO: 95) |

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs functional domain (e.g., nuclease such as Cas) to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

Non-limiting examples of nucleases include meganucleases, TALENs and zinc finger nucleases. The nuclease may comprise heterologous DNA-binding and cleavage domains (e.g., zinc finger nucleases; meganuclease DNA-binding domains with heterologous cleavage domains) or, alternatively, the DNA-binding domain of a naturally-occurring nuclease may be altered to bind to a selected target site (e.g., a meganuclease that has been engineered to bind to site different than the cognate binding site).

### Expression of Chimeric Antigen Receptor or T-Cell Receptor

The engineered cells, in particular immune cells, such as T cells, NK cells and other immune cell types, can also be genetically engineered with vectors designed to express CARs that redirect cytotoxicity toward tumor cells. CARs are molecules that combine antibody-based specificity for a target antigen (e.g., tumor antigen) with a T cell receptor-activating intracellular domain to generate a chimeric protein that exhibits a specific anti-tumor cellular immune activity.

The CARs contemplated herein comprise an extracellular domain that binds to a specific target antigen (also referred to as a binding domain or antigen-specific binding domain), a transmembrane domain and an intracellular signaling domain. A characteristic of CARs is their ability to redirect immune effector cell specificity, thereby triggering proliferation, cytokine production, phagocytosis or production of molecules that may mediate cell death of the target antigen expressing cell in a major histocompatibility (MHC) independent manner, exploiting the cell specific targeting abilities of monoclonal antibodies, soluble ligands or cell specific co-receptors.

In some embodiments, a CAR comprises an extracellular binding domain including but not limited to an antibody or antigen binding fragment thereof, a tethered ligand, or the extracellular domain of a co-receptor, that specifically binds a target antigen.

By way of non-limiting examples, target antigens may include: HPV oncoproteins, including HPV-16 E6 and HPV-16 E7, alpha folate receptor, 5T4, αᵥβᵥ integrin, BCMA, TACI, B7-H3, B7-H6, CAIX, CD19, CD20, CD22, CD28, CD30, CD33, CD44, CD44v6, CD44v7/8, CD70, CD79a, CD79b, CD123, CD137 (4-1BB), CD138, CD171, CEA, CSPG4, CLL-1, EGFR, EGFR family including ErbB2 (HERII), EGFRvIII, EGP2, EGP40, EPCAM, EphA2, EpCAM, FAP, fetal AchR, FRa, GD2, GD3, Glypican-3 (GPC3), HLA-Al+MAGEI, HLA-A2+MAGE1, HLAA3+MAGE1, HLA-Al+NY-ES0-1, HLA-A2+NY-ES0-1, HLA-A3+NY-ES0-1, IL-llRa, IL-13Ra2, Lambda, Lewis-Y, Kappa, Mesothelin, Mud, Muc16, NCAM, NKG2D Ligands, NYE-S0-1, PRAME, PSCA, PSMA, RORI, SSX, Survivin, TAG72, TEMs, and VEGFRII; one or more hinge domains or spacer domains; a transmembrane domain including, but not limited to, transmembrane domains from CD8a, CD4, CD45, PD-1, and CD152; one or more intracellular costimulatory signaling domains including but not limited to intracellular costimulatory signaling domains from CD28, CD54 (ICAM), CD134 (OX40), CD137 (41BB), CD152 (CTLA4), CD273 (PD-L2), CD274 (PD-L1), and CD278 (ICOS); and a primary signaling domain from CD3ζ or FcRγ. In one embodiment described herein, the CAR binds to a tumor antigen comprising CLL-1, CD19, CD20, CD28, CD137 (4-1BB), Glypican-3 (GPC3), PSCA or PSMA. In a certain embodiment, the CAR binds CD19. In a certain embodiment, the CAR binds CD20. In a certain embodiment, the CAR includes a first scFv that binds CD19 and a second scFv that binds CD20. Example CD19- or CD20-binding sequences are provided in Table 3.

TABLE 3
Example Antigen-Binding Sequences

| Name | Sequence |
|---|---|
| Anti-CD20 | SEQ ID NO: 1 |
| v01 VH/VL | QVQLQQWGAGLLKPSETLSLTCAVYGGSFSGYYWSWIRQPPGKGLEWIG |
| | EIDHSGSTNYNPSLKSRVTISVDTSKNQFSLKLSSVTAADTAVYYCARGG |
| | GSWYSNWFDPWGQGTMVTVSS |
| | SEQ ID NO: 2 |
| | DIQMTQSPSTLSASVGDRVTITCRASQSISSWLAWYQQKPGKAPKLLIYD |
| | ASSLESGVPSRFSGSGSGTEFTLTISSLQPDDFATYYCQQDRSLPPTFGGGT |
| | KVEIK |
| Anti-CD20 | SEQ ID NO: 3 |
| v02 VH/VL | QVQLQQWGAGLLKPSETLSLTCAVYGGSFSGIHWNWIRQPPGKGLEWIG |
| | DIDTSGSTNYNPSLKSRVTISVDTSKNQFSLKLSSVTAADTAVYYCARLGQ |
| | ESATYLGMDVWGQGTTVTVSS |
| | SEQ ID NO: 4 |
| | DIVMTQSPDSLAVSLGERATINCKSSQSVLYSSNNKNYLAWYQQKPGQPP |
| | KLLIYWASTRESGVPDRFSGSGSGTDFTLTISSLQAEDVAVYYCQQLYTYP |
| | FTFGGGTKVEIK |
| Anti-CD20 | SEQ ID NO: 5 |
| v03 VH/VL | QLQLQESGPGLVKPSETLSLTCTVSGGSISSSSYYWGWIRQPPGKGLEWIG |
| | SIYYSGSTYYNPSLKSRVTISVDTSKNQFSLKLSSVTAADTAVYYCARETD |
| | YSSGMGYGMDVWGQGTTVTVSS |
| | SEQ ID NO: 6 |
| | DIQMTQSPSSLSASVGDRVTITCRASQSINSYLNWYQQKPGKAPKLLIYAA |
| | SSLQSGVPSRFSGSGSGTDFTLTISSLQPEDFATYYCQQSLADPFTFGGGTK |
| | VEIK |
| Anti-CD20 | SEQ ID NO: 7 |
| v04 VH/VL | QVQLVQSGAEVKKPGASVKVSCKASGYTFKEYGISWVRQAPGQGLEWM |
| | GWISAYSGHTYYAQKLQGRVTMTTDTSTSTAYMELRSLRSDDTAVYYC |
| | ARGPHYDDWSGFIIWFDPWGQGTLVTVSS |
| | SEQ ID NO: 8 |
| | DIQMTQSPSSLSASVGDRVTITCRASQSISSYLNWYQQKPGKAPKLLIYAA |
| | SSLQSGVPSRFSGSGSGTDFTLTISSLQPEDFATYYCQQSYRFPPTFGQGTK |
| | VEIK |
| Anti-CD20 | SEQ ID NO: 9 |
| v05 VH/VL | QVQLQESGPGLVKPSETLSLTCTVSGGSISSPDHYWGWIRQPPGKGLEWI |
| | GSIYASGSTFYNPSLKSRVTISVDTSKNQFSLKLSSVTAADTAVYYCARET |
| | DYSSGMGYGMDVWGQGTTVTVSS |
| | SEQ ID NO: 10 |
| | DIQMTQSPSSLSASVGDRVTITCRASQSINSYLNWYQQKPGKAPKLLIYAA |
| | SSLQSGVPSRFSGSGSGTDFTLTISSLQPEDFATYYCQQSLADPFTFGGGTK |
| | VEIK |
| Anti-CD20 | SEQ ID NO: 11 |
| v06 VH/VL | QITLKESGPTLVKPTQTLTLTCTFSGFSLDTEGVGVGWIRQPPGKALEWLA |
| | LIYFNDQKRYSPSLKSRLTITKDTSKNQVVLTMTNMDPVDTAVYYCARD |
| | TGYSRWYYGMDVWGQGTTVTVSS |
| | SEQ ID NO: 12 |
| | DIQMTQSPSSVSASVGDRVTITCRASQGISSWLAWYQQKPGKAPKLLIYA |
| | ASSLQSGVPSRFSGSGSGTDFTLTISSLQPEDFATYYCQQAYAYPITFGGGT |
| | KVEIK |
| Anti-CD20 | SEQ ID NO: 13 |
| v07 VH/VL | QVQLQQWGAGLLKPSETLSLTCAVYGGSFEKYYWSWIRQPPGKGLEWIG |
| | EIYHSGLTNYNPSLKSRVTISVDTSKNQFSLKLSSVTAADTAVYYCARVR |
| | YDSSDSYYYSYDYGMDVWGQGTTVTVSS |
| | SEQ ID NO: 14 |
| | DIVLTQSPDSLAVSLGERATINCKSSQSVLYSSNNKNYLAWYQQKPGQPP |
| | KLLIYWASSRESGVPDRFSGSGSGTDFTLTISSLQAEDVAVYYCQQSYSFP |
| | WTFGGGTKVEIK |
| Anti-CD20 | SEQ ID NO: 15 |
| v08 VH/VL | QVQLQQWGAGLLKPSETLSLTCAVYGGSFSRYVWSWIRQPPGKGLEWIG |
| | EIDSSGKTNYNPSLKSRVTISVDTSKNQFSLKLSSVTAADTAVYYCARVR |
| | YDSSDSYYYSYDYGMDVWGQGTTVTVSS |
| | SEQ ID NO: 16 |
| | DIVLTQSPDSLAVSLGERATINCKSSQSVLYSSNNKNYLAWYQQKPGQPP |
| | KLLIYWASSRESGVPDRFSGSGSGTDFTLTISSLQAEDVAVYYCQQSYSFP |
| | WTFGGGTKVEIK |
| Anti-CD20 | SEQ ID NO: 17 |
| v09 VH/VL | QVQLQQWGAGLLKPSETLSLTCAVYGGSFSGYAWSWIRQPPGKGLEWIG |
| | EIDHRGFTNYNPSLKSRVTISVDTSKNQFSLKLSSVTAADTAVYYCARVR |
| | YDSSDSYYYSYDYGMDVWGQGTTVTVSS |
| | SEQ ID NO: 18 |
| | DIVLTQSPDSLAVSLGERATINCKSSQSVLYSSNNKNYLAWYQQKPGQPP |
| | KLLIYWASSRESGVPDRFSGSGSGTDFTLTISSLQAEDVAVYYCQQSYSFP |
| | WTFGGGTKVEIK |
| Anti-CD20 | SEQ ID NO: 19 |
| v10 VH/VL | QVQLQQWGAGLLKPSETLSLTCAVYGGSFQKYYWSWIRQPPGKGLEWIG |
| | EIDTSGFTNYNPSLKSRVTISVDTSKNQFSLKLSSVTAADTAVYYCARVGR |
| | YSYGYYITAFDIWGQGTTVTVSS |
| | SEQ ID NO: 20 |
| | DIVMTQSPDSLAVSLGERATINCKSSQSVLYSSNNKNYLAWYQQKPGQPP |
| | KLLIYWASTRESGVPDRFSGSGSGTDFTLTISSLQAEDVAVYYCQQHYSFP |
| | FTFGGGTKVEIK |
| Anti-CD19 | SEQ ID NO: 21 |
| VH/VL v01 | EVKLQESGPGLVAPSQSLSVTCTVSGVSLPDYGVSWIRQPPRKGLEWLGV |
| | IWGSETTYYNSALKSRLTIIKDNSKSQVFLKMNSLQTDDTAIYYCAKHYY |
| | YGGSYAMDYWGQGTSVTVSS |
| | SEQ ID NO: 22 |
| | DIQMTQTTSSLSASLGDRVTISCRASQDISKYLNWYQQKPDGTVKLLIYHT |
| | SRLHSGVPSRFSGSGSGTDYSLTISNLEQEDIATYFCQQGNTLPYTFGGGT |
| | KLEIT |
| Anti-CD19 | SEQ ID NO: 23 |
| VH/VL v02 | EVOLVESGGGLVQPGRSLRLSCTASGVSLPDYGVSWIRQPPGKGLEWIGV |
| | IWGSETTYYNSALKSRFTISRDNSKNTLYLQMNSLRAEDTAVYYCAKHY |
| | YYGGSYAMDYWGQGTLVTVSS |
| | SEQ ID NO: 24 |
| | DIQMTQSPSSLSASVGDRVTITCRASQDISKYLNWYQQKPDQAPKLLIKHT |
| | SRLHSGVPSRFSGSGSGTDYTLTISSLQPEDFATYYCQQGNTLPYTFGQGT |
| | KLEIK |
| Anti-CD19 | SEQ ID NO: 25 |
| scFv | DIQMTQTTSSLSASLGDRVTISCRASQDISKYLNWYQQKPDGTVKLLIYHT |
| | SRLHSGVPSRFSGSGSGTDYSLTISNLEQEDIATYFCQQGNTLPYTFGGGT |
| | KLEITGSTSGSGKPGSGEGSTKGEVKLQESGPGLVAPSQSLSVTCTVSGVS |
| | LPDYGVSWIRQPPRKGLEWLGVIWGSETTYYNSALKSRLTIIKDNSKSQV |
| | FLKMNSLQTDDTAIYYCAKHYYYGGSYAMDYWGQGTSVTVSS |
| Anti- | SEQ ID NO: 26 |
| CD20/anti- | MLLLVTSLLLCELPHPAFLLIPDIQMTQTTSSLSASLGDRVTISCRASQDISK |
| CD19 | YLNWYQQKPDGTVKLLIYHTSRLHSGVPSRFSGSGSGTDYSLTISNLEQE |
| bicistronic | DIATYFCQQGNTLPYTFGGGTKLEITGGGGSGGGGSGGGGSEVKLQESGP |
| CAR v01 | GLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMD |
| | YWGQGTSVTVSSAAALDNEKSNGTIIHVKGKHLCPSPLFPGPSKPFWVLV |
| | VVGGVLACYSLLVTVAFIIFWVRSKRSRLLHSDYMNMTPRRPGPTRKHY |
| | QPYAPPRDFAAYRSRVKFSRSADAPAYQQGQNQLYNELNLGRREEYDVL |
| | DKRRGRDPEMGGKPRRKNPQEGLYNELQKDKMAEAYSEIGMKGERRRG |
| | KGHDGLYQGLSTATKDTYDALHMQALPPRRAKRSGSGEGRGSLLTCGD |
| | VEENPGPMALPVTALLLPLALLLHAARPQLQLQESGPGLVKPSETLSLTCT |
| | VSGGSISSSSYYWGWIRQPPGKGLEWIGSIYYSGSTYYNPSLKSRVTISVD |
| | TSKNQFSLKLSSVTAADTAVYYCARETDYSSGMGYGMDVWGQGTTVTV |
| | SSGSTSGSGKPGSGEGSTKGDIQMTQSPSSLSASVGDRVTITCRASQSINSY |
| | LNWYQQKPGKAPKLLIYAASSLQSGVPSRFSGSGSGTDFTLTISSLQPEDF |
| | ATYYCQQSLADPFTFGGGTKVEIKAAAFVPVFLPAKPTTTPAPRPPTPAPTI |
| | ASQPLSLRPEACRPAAGGAVHTRGLDFACDIYIWAPLAGTCGVLLLSLVIT |
| | LYCNHRNRFSVVKRGRKKLLYIFKQPFMRPVQTTQEEDGCSCRFPEEEEG |
| | GCELRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQ |
| | GLSTATKDTYDALHMQALPPR |
| Anti- | SEQ ID NO: 27 |
| CD20/anti- | MLLLVTSLLLCELPHPAFLLIPDIQMTQSPSSLSASVGDRVTITCRASQSINS |
| CD19 | YLNWYQQKPGKAPKLLIYAASSLQSGVPSRFSGSGSGTDFTLTISSLQPED |
| bispecific | FATYYCQQSLADPFTFGGGTKVEIKGGGGSGKPGSGEGGSQLQLQESGPG |
| CAR v02 | LVKPSETLSLTCTVSGGSISSSSYYWGWIRQPPGKGLEWIGSIYYSGSTYY |
| | NPSLKSRVTISVDTSKNQFSLKLSSVTAADTAVYYCARETDYSSGMGYG |
| | MDVWGQGTTVTVSSGGGGSGKPGSDIQMTQSPSSLSASVGDRVTITCRAS |
| | QDISKYLNWYQQKPDQAPKLLIKHTSRLHSGVPSRFSGSGSGTDYTLTISS |
| | LQPEDFATYYCQQGNTLPYTFGQGTKLEIKGGGGSGGGGSGGGGSEVQL |
| | VESGGGLVQPGRSLRLSCTASGVSLPDYGVSWIRQPPGKGLEWIGVIWGS |
| | ETTYYNSALKSRFTISRDNSKNTLYLQMNSLRAEDTAVYYCAKHYYYGG |
| | SYAMDYWGQGTLVTVSSAAALDNEKSNGTIIHVKGKHLCPSPLFPGPSKP |
| | FWVLVVVGGVLACYSLLVTVAFIIFWVRSKRSRLLHSDYMNMTPRRPGP |
| | TRKHYQPYAPPRDFAAYRSRVKFSRSADAPAYQQGQNQLYNELNLGRRE |
| | EYDVLDKRRGRDPEMGGKPRRKNPQEGLYNELQKDKMAEAYSEIGMKG |
| | ERRRGKGHDGLYQGLSTATKDTYDALHMQALPPR |
| Anti- | SEQ ID NO: 28 |
| CD20/anti- | ATGCTGCTGCTGGTGACATCTCTGCTGCTTTGCGAGCTGCCCCACCCTG |
| CD19 | CCTTCCTGCTTATCCCCGACATTCAGATGACCCAGACCACCAGCAGCC |
| bicistronic | TGAGCGCCAGCTTAGGAGATAGAGTTACCATCAGCTGCAGAGCCAGC |
| CAR v01 | CAGGACATCAGCAAATACCTGAACTGGTATCAGCAGAAGCCCGACGG |
| | CACTGTGAAACTGCTTATTTACCACACCTCCAGACTGCACAGCGGCGT |
| | TCCCAGCAGATTCTCTGGCAGCGGATCTGGAACCGACTACAGCCTCAC |
| | CATCTCCAACCTGGAGCAGGAGGACATCGCCACCTACTTCTGCCAGCA |
| | GGGCAACACACTGCCCTACACCTTCGGAGGAGGAACCAAGCTGGAGA |
| | TCACCGGGGGAGGAGGCTCTGGAGGCGGCGGATCAGGAGGAGGGGG |
| | ATCTGAGGTTAAGCTGCAGGAGAGCGGCCCTGGCCTGGTGGCTCCTAG |
| | CCAATCTTTATCTGTGACCTGCACTGTGTCCGGCGTTAGCCTGCCCGAT |
| | TATGGCGTTTCCTGGATCAGACAGCCCCCCAGAAAGGGCCTGGAATGG |
| | CTGGGCGTTATCTGGGGCAGCGAGACCACATACTACAACAGCGCCCTG |
| | AAGAGCAGACTTACGATTATCAAGGACAACAGCAAGAGCCAGGTTTT |
| | CCTGAAGATGAACAGCCTGCAGACCGACGACACCGCCATCTACTACTG |
| | CGCTAAGCACTACTACTACGGCGGCAGCTACGCCATGGACTACTGGGG |
| | CCAGGGAACAAGCGTTACCGTTAGCAGCGCTGCTGCACTGGACAACG |
| | AGAAGAGCAACGGCACCATCATCCACGTTAAGGGCAAGCACCTGTGC |
| | CCCAGCCCTCTGTTCCCTGGACCTTCTAAGCCTTTCTGGGTTCTGGTGG |
| | TGGTCGGCGGCGTTTTAGCCTGTTACAGCCTTCTGGTGACTGTGGCCTT |
| | CATCATCTTTTGGGTTAGAAGCAAGAGAAGCAGACTGCTCCACAGCGA |
| | CTACATGAACATGACCCCCAGACGGCCTGGCCCCACCAGAAAGCATT |
| | ACCAGCCCTACGCTCCTCCCAGAGACTTCGCCGCCTACAGGAGCAGAG |
| | TTAAATTCAGCAGATCCGCCGATGCCCCCGCTTACCAACAGGGACAAA |
| | ACCAGCTGTACAATGAGCTCAACCTGGGGAGAAGAGAAGAATACGAC |
| | GTTCTGGATAAGAGAAGGGGCAGAGATCCCGAAATGGGGGGCAAGCC |
| | CAGACGCAAGAACCCTCAGGAGGGGCTTTACAACGAACTGCAGAAGG |
| | ATAAGATGGCTGAGGCTTACTCGGAGATTGGGATGAAGGGGGAGAGA |
| | AGGCGGGGCAAGGGACACGATGGCTTATACCAGGGGCTGAGCACCGC |
| | CACCAAGGACACATACGACGCTCTTCATATGCAGGCTCTGCCCCCAAG |
| | AAGGGCTAAGAGATCTGGCTCTGGCGAGGGCAGAGGCAGCTTGCTTA |
| | CATGTGGCGATGTGGAGGAGAACCCCGGGCCCATGGCTCTTCCTGTGA |
| | CAGCTCTTCTGCTGCCCCTGGCCCTGCTTCTGCATGCTGCTAGACCTCA |
| | GCTTCAGCTCCAAGAGAGCGGACCTGGCTTAGTGAAGCCCAGCGAAA |
| | CCCTGTCCCTCACCTGCACCGTTTCTGGCGGAAGCATCAGCAGCTCCA |
| | GCTATTACTGGGGATGGATCAGGCAGCCCCCTGGCAAGGGTTTAGAAT |
| | GGATCGGCTCGATATATTACTCCGGCAGCACCTACTATAACCCCAGCT |
| | TGAAGAGCCGGGTTACCATTTCTGTGGACACATCAAAGAACCAGTTCA |
| | GCCTGAAGCTGAGCTCTGTGACTGCCGCCGACACAGCTGTGTACTACT |
| | GTGCCAGAGAGACAGACTACTCCAGCGGCATGGGCTACGGCATGGAT |
| | GTGTGGGGACAAGGAACCACCGTTACTGTGAGCAGCGGTTCCACCAG |
| | CGGCTCAGGCAAGCCTGGCTCAGGAGAAGGAAGCACCAAGGGGGATA |
| | TACAGATGACACAGAGCCCCTCCAGCCTGTCCGCCAGCGTTGGCGATC |
| | GTGTAACGATCACCTGCCGGGCCTCTCAGAGCATCAACTCCTACCTCA |
| | ATTGGTATCAACAGAAGCCAGGCAAGGCCCCCAAATTACTCATCTACG |
| | CCGCCAGCAGCTTACAGAGCGGGGTTCCCTCTAGATTCTCCGGCTCCG |
| | GTTCTGGAACAGATTTCACCCTCACTATCTCCAGCTTGCAGCCCGAGG |
| | ATTTCGCCACTTATTACTGTCAGCAGAGCCTGGCCGACCCCTTCACATT |
| | CGGCGGAGGCACAAAGGTTGAGATCAAGGCAGCTGCTTTCGTGCCTGT |
| | GTTCCTGCCTGCTAAGCCCACCACCACTCCTGCTCCAAGACCTCCTACC |
| | CCCGCTCCTACAATCGCCAGCCAACCTCTGAGCCTGAGACCGGAGGCA |
| | TGCAGACCTGCGGCAGGGGGAGCAGTTCACACAAGAGGCTTGGACTT |
| | CGCTTGCGACATCTACATCTGGGCCCCTCTGGCCGGCACATGCGGAGT |
| | TCTTCTTCTTAGCCTGGTGATCACCCTGTACTGCAACCACAGAAACAG |
| | ATTCAGCGTTGTGAAGAGAGGCCGGAAGAAGCTGCTGTACATCTTCAA |
| | GCAGCCCTTCATGAGACCTGTGCAGACCACACAGGAGGAAGACGGCT |
| | GCAGCTGTAGATTCCCCGAGGAAGAGGAGGGCGGCTGTGAGCTGAGA |
| | GTTAAGTTCAGCAGGAGCGCCGACGCCCCTGCCTACCAGCAAGGACA |
| | GAATCAACTGTACAACGAGCTGAACCTGGGCAGACGGGAGGAATACG |
| | ATGTGCTGGACAAGAGGAGAGGCAGAGACCCCGAGATGGGCGGCAA |
| | ACCTAGAAGAAAGAACCCCCAGGAGGGCCTGTATAACGAGCTCCAGA |
| | AGGACAAGATGGCCGAGGCCTACAGCGAGATCGGCATGAAGGGCGAA |
| | AGAAGAAGAGGCAAGGGCCACGACGGCCTCTACCAGGGCTTAAGCAC |
| | AGCTACAAAGGACACCTACGACGCCCTGCACATGCAGGCCCTGCCCCC |
| | TAGATGA |
| Anti- | SEQ ID NO: 29 |
| CD20/anti- | ATGCTTCTCCTGGTGACAAGCCTTCTGCTCTGTGAGTTACCACACCCAG |
| CD19 | CATTCCTCCTGATCCCAGACATCCAGATGACCCAGTCTCCATCCTCCCT |
| bispecific | GTCTGCATCTGTAGGAGACAGAGTCACCATCACTTGCCGGGCAAGTCA |
| CAR v02 | GAGCATTAACAGCTATTTAAATTGGTATCAGCAGAAACCAGGGAAAG |
| | CCCCTAAGCTCCTGATCTATGCTGCATCCAGTTTGCAAAGTGGGGTCC |
| | CATCAAGGTTCAGTGGCAGTGGATCTGGGACAGATTTCACTCTCACCA |
| | TCAGCAGTCTGCAACCTGAAGATTTTGCAACTTACTACTGCCAGCAAA |
| | GCCTCGCCGACCCTTTCACTTTTGGCGGAGGGACCAAGGTTGAGATCA |
| | AAGGGGGGGGTGGAAGTGGGAAGCCTGGCAGCGGCGAGGGCGGCAG |
| | TCAGCTGCAGCTGCAGGAGTCGGGCCCAGGACTGGTGAAGCCTTCGG |
| | AGACCCTGTCCCTCACCTGCACTGTCTCTGGTGGCTCCATCAGCAGTA |
| | GTAGTTACTACTGGGGCTGGATCCGCCAGCCCCCAGGGAAGGGGCTG |
| | GAGTGGATTGGGAGTATCTATTATAGTGGGAGCACCTACTACAACCCG |
| | TCCCTCAAGAGTCGAGTCACCATATCCGTAGACACGTCCAAGAACCAG |
| | TTCTCCCTGAAGCTGAGTTCTGTGACCGCCGCAGACACGGCGGTGTAC |
| | TACTGCGCCAGAGAGACTGACTACAGCAGCGGAATGGGATACGGAAT |
| | GGACGTATGGGGCCAGGGAACAACTGTCACCGTCTCCTCAGGCGGTG |
| | GCGGCAGTGGGAAGCCTGGCAGCGATATTCAAATGACCCAGTCCCCG |
| | TCCTCCCTGAGTGCCTCCGTCGGTGACCGTGTTACGATTACCTGCCGTG |
| | CGAGCCAAGACATCTCTAAATACCTGAACTGGTATCAGCAAAAACCG |
| | GATCAGGCACCGAAACTGCTGATCAAACATACCTCACGTCTGCACTCG |
| | GGTGTGCCGAGCCGCTTTAGTGGTTCCGGCTCAGGTACCGATTACACC |
| | CTGACGATCAGCTCTCTGCAGCCGGAAGACTTTGCCACGTATTACTGC |
| | CAGCAAGGTAATACCCTGCCGTATACGTTCGGCCAAGGTACCAAACTG |
| | GAAATCAAAGGGGGGGGTGGAAGTGGGGGCGGTGGCAGCGGCGGTG |
| | GCGGCAGTGAAGTGCAGCTGGTTGAAAGCGGTGGTGGTCTGGTTCAA |
| | CCGGGTCGTTCCCTGCGTCTGTCATGTACGGCGAGTGGTGTCTCCCTGC |
| | CGGACTATGGCGTGTCCTGGATTCGTCAGCCGCCGGGTAAAGGCCTGG |
| | AATGGATTGGTGTCATCTGGGGCAGTGAAACCACGTATTACAACTCGG |
| | CCCTGAAAAGCCGTTTCACCATCTCTCGCGATAACAGTAAAAATACGC |
| | TGTACCTGCAGATGAATAGCCTGCGCGCGGAAGACACCGCCGTTTACT |
| | ACTGCGCAAAACATTACTACTACGGTGGCAGCTATGCTATGGATTACT |
| | GGGGTCAAGGCACGCTGGTCACCGTTTCGTCAGCCGCTGCCCTAGACA |
| | ATGAGAAGAGCAATGGAACCATTATCCATGTGAAAGGGAAACACCTT |
| | TGTCCAAGTCCCCTATTTCCCGGACCTTCTAAGCCCTTTTGGGTGCTGG |
| | TGGTGGTTGGGGGAGTCCTGGCTTGCTATAGCTTGCTAGTAACAGTGG |
| | CCTTTATTATTTTCTGGGTGAGGAGTAAGAGGAGCAGGCTCCTGCACA |
| | GTGACTACATGAACATGACTCCCCGCCGCCCCGGGCCCACCCGCAAGC |
| | ATTACCAGCCCTATGCCCCACCACGCGACTTCGCAGCCTATCGCTCCA |
| | GAGTGAAGTTCAGCAGGAGCGCAGACGCCCCCGCGTACCAGCAGGGC |
| | CAGAACCAGCTCTATAACGAGCTCAATCTAGGACGAAGAGAGGAGTA |
| | CGATGTTTTGGACAAGAGACGTGGCCGGGACCCTGAGATGGGGGGAA |
| | AGCCGAGAAGGAAGAACCCTCAGGAAGGCCTGTACAATGAACTGCAG |
| | AAAGATAAGATGGCGGAGGCCTACAGTGAGATTGGGATGAAAGGCGA |
| | GCGCCGGAGGGGCAAGGGGCACGATGGCCTTTACCAGGGTCTCAGTA |
| | CAGCCACCAAGGACACCTACGACGCCCTTCACATGCAGGCCCTGCCCC |
| | CTCGATGA |
| anti-CD19 | SEQ ID NO: 30 |
| CAR | DIQMTQTTSSLSASLGDRVTISCRASQDISKYLNWYQQKPDGTVKLLIYHT |
| | SRLHSGVPSRFSGSGSGTDYSLTISNLEQEDIATYFCQQGNTLPYTFGGGT |
| | KLEITGSTSGSGKPGSGEGSTKGEVKLQESGPGLVAPSQSLSVTCTVSGVS |
| | LPDYGVSWIRQPPRKGLEWLGVIWGSETTYYNSALKSRLTIIKDNSKSQV |
| | FLKMNSLQTDDTAIYYCAKHYYYGGSYAMDYWGQGTSVTVSSAAAIEV |
| | MYPPPYLDNEKSNGTIIHVKGKHLCPSPLFPGPSKPFWVLVVVGGVLACY |
| | SLLVTVAFIIFWVRSKRSRLLHSDYMNMTPRRPGPTRKHYQPYAPPRDFA |
| | AYRSRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQ |
| | GLSTATKDTYDALHMQALPPR |

In various embodiments a binding motif may comprise a heavy chain variable domain of the present disclosure (e.g., having at least 75% sequence identity to a heavy chain variable domain shown in Table 3, e.g., at least 80%, 85%, 90%, 95%, or 100% identity; e.g., 85-90%, 85-95%, 85-100%, 90-95%, 90-100%, or 95-100%), a light chain variable domain of the present disclosure (e.g., having at least 75% sequence identity to a light chain variable domain shown in Table 3 e.g., at least 80%, 85%, 90%, 95%, or 100% identity; e.g., 85-90%, 85-95%, 85-100%, 90-100%, or 95-100%), and a linker (see, e.g., Whitlow et al. Protein Eng. 1993 November; 6(8):989-95.). In various embodiments a binding motif may comprise a leader sequence or a signal sequence, a heavy chain variable domain of the present disclosure (e.g., having at least 75% sequence identity to a heavy chain variable domain shown in Table 3, e.g., at least 80%, 85%, 90%, 95%, or 100% identity; e.g., 85-90%, 85-95%, 85-100%, 90-95%, 90-100%, or 95-100%), a light chain variable domain of the present disclosure (e.g., having at least 75% sequence identity to a light chain variable domain shown in Table 3, e.g., at least 80%, 85%, 90%, 95%, or 100% identity; e.g., 85-90%, 85-95%, 85-100%, 90-95%, 90-100%, or 95-100%), and a linker.

If provided with an amino acid or nucleotide sequence of a binding motif comprising a heavy chain variable domain of the present disclosure and a light chain variable domain of the present disclosure, the linker joining the two variable domains will be apparent from the sequence in view of the present disclosure and knowledge in the art. If provided with an amino acid or nucleotide sequence of a binding motif comprising a heavy chain variable domain of the present disclosure and a light chain variable domain of the present disclosure, the leader sequence will be apparent in view of the present disclosure and knowledge in the art. For the avoidance of doubt, a heavy chain variable domain and a light chain variable domain of the present disclosure may be present in any orientation, e.g., an orientation in which the heavy chain variable domain is C terminal of the light chain variable domain or in which the heavy chain variable domain is N terminal of the light chain variable domain. Exemplary anti-CD20 binding motifs are shown in Table 4.

TABLE 4
Exemplary Anti-CD20 Binding Motif Sequences

| Name | Sequence |
|---|---|
| (Ab1) | SEQ ID NO: 31 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCAGCGGCTATTACTGGAGCTGGATCCGGCAGCCTCCTGGAAAAGGA |
| | TTAGAATGGATCGGCGAGATAGACCACAGCGGGAGCACAAACTACAACCCC |
| | AGCCTGAAATCGCGGGTTACAATCTCTGTGGACACAAGCAAGAATCAGTTC |
| | TCCCTGAAGCTGAGCAGCGTTACTGCCGCCGACACAGCTGTGTACTATTGCG |
| | CCAGAGGCGGAGGCTCCTGGTACAGCAACTGGTTCGATCCTTGGGGCCAAG |
| | GCACCATGGTGACCGTTTCCAGCGGCTCTACAAGCGGCAGCGGGAAACCTG |
| | GTTCTGGAGAGGGCAGCACAAAGGGCGACATCCAGATGACACAGAGCCCC |
| | AGCACCCTTAGCGCCTCTGTGGGAGATAGGGTTACCATTACCTGCAGGGCTT |
| | CCCAGAGCATCAGCAGCTGGCTGGCATGGTATCAACAGAAGCCTGGCAAGG |
| | CTCCCAAGCTGCTCATCTATGACGCCTCCAGCCTGGAAAGCGGGGTTCCCTC |
| | CAGATTTAGCGGCTCAGGCTCCGGAACAGAGTTCACCCTTACCATCTCTAGC |
| | CTGCAACCCGACGACTTCGCTACTTATTACTGTCAACAAGACAGAAGCTTGC |
| | CCCCCACATTCGGCGGAGGGACCAAGGTTGAGATCAAG |
| (Ab2) | SEQ ID NO: 32 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCAGCGGCATCCACTGGAACTGGATCCGGCAGCCTCCTGGCAAAGGC |
| | CTTGAATGGATCGGCGATATCGACACCAGCGGCTCCACCAACTACAACCCC |
| | AGCCTGAAATCGAGGGTTACAATCTCTGTGGACACAAGCAAGAATCAGTTC |
| | TCCCTGAAGCTGAGCAGCGTTACTGCCGCCGACACAGCTGTGTACTATTGCG |
| | CCAGACTGGGCCAGGAAAGCGCTACCTACCTTGGCATGGATGTGTGGGGGC |
| | AGGGCACCACCGTTACTGTTAGCTCTGGCTCAACAAGCGGCAGCGGCAAGC |
| | CTGGCTCAGGAGAAGGAAGCACAAAGGGCGACATTGTAATGACTCAGAGC |
| | CCCGACAGCCTGGCCGTTAGCTTAGGCGAAAGGGCTACAATCAATTGCAAG |
| | AGCAGCCAGAGCGTTCTGTACAGCAGCAACAACAAGAACTACCTCGCATGG |
| | TATCAACAGAAGCCAGGCCAGCCTCCCAAGCTGCTCATCTACTGGGCTTCCA |
| | CCAGAGAGAGCGGGGTTCCCGATAGATTCTCCGGCTCCGGTTCTGGAACAG |
| | ATTTCACGCTCACAATCAGCAGCTTACAGGCCGAGGATGTGGCTGTCTACTA |
| | TTGTCAGCAGTTGTACACCTACCCCTTCACATTCGGCGGAGGCACCAAGGTT |
| | GAGATCAAG |
| (Ab3) | SEQ ID NO: 33 |
| | ggtaccCCCGGgCCCATGGCTCTTCCTGTGACAGCTCTTCTGCTGCCCCTGGCCC |
| | TGCTTCTGCATGCTGCTAGACCTCAGCTTCAGCTCCAAGAGAGCGGACCTGG |
| | CTTAGTGAAGCCCAGCGAAACCCTGTCCCTCACCTGCACCGTTTCTGGCGGA |
| | AGCATCAGCAGCTCCAGCTATTACTGGGGATGGATCAGGCAGCCCCCTGGC |
| | AAGGGTTTAGAATGGATCGGCTCGATATATTACTCCGGCAGCACCTACTATA |
| | ACCCCAGCTTGAAGAGCCGGGTTACCATTTCTGTGGACACATCAAAGAACC |
| | AGTTCAGCCTGAAGCTGAGCTCTGTGACTGCCGCCGACACAGCTGTGTACTA |
| | CTGTGCCAGAGAGACAGACTACTCCAGCGGCATGGGCTACGGCATGGATGT |
| | GTGGGGACAAGGAACCACCGTTACTGTGAGCAGCGGTTCCACCAGCGGCTC |
| | AGGCAAGCCTGGCTCAGGAGAAGGAAGCACCAAGGGGGATATACAGATGA |
| | CACAGAGCCCCTCCAGCCTGTCCGCCAGCGTTGGCGATCGTGTAACGATCA |
| | CCTGCCGGGCCTCTCAGAGCATCAACTCCTACCTCAATTGGTATCAACAGAA |
| | GCCAGGCAAGGCCCCCAAATTACTCATCTACGCCGCCAGCAGCTTACAGAG |
| | CGGGGTTCCCTCTAGATTCTCCGGCTCCGGTTCTGGAACAGATTTCACCCTC |
| | ACTATCTCCAGCTTGCAGCCCGAGGATTTCGCCACTTATTACTGTCAGCAGA |
| | GCCTGGCCGACCCCTTCACATTCGGCGGAGGCACAAAGGTTGAGATCAAG |
| (Ab4) | SEQ ID NO: 34 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGCTTGTGCAGAGCGGAGCTGA |
| | AGTTAAGAAGCCTGGCGCCTCTGTGAAGGTTAGCTGCAAGGCCAGCGGCTA |
| | CACATTCAAGGAATATGGCATCTCCTGGGTTAGGCAGGCTCCCGGCCAAGG |
| | CTTAGAATGGATGGGCTGGATCTCCGCCTACTCCGGCCACACCTACTACGCC |
| | CAGAAGCTTCAGGGCAGGGTTACCATGACCACCGACACCAGCACCTCTACC |
| | GCCTATATGGAGCTGAGGAGCCTGAGATCGGACGACACAGCTGTGTATTAC |
| | TGCGCCAGAGGCCCCCACTACGACGACTGGTCTGGATTTATCATCTGGTTCG |
| | ACCCCTGGGGGCAGGGCACCCTGGTCACAGTTTCTTCTGGCTCCACCAGCGG |
| | AAGCGGCAAGCCAGGCTCAGGCGAAGGATCTACAAAAGGCGACATCCAAA |
| | TGACACAGAGCCCCAGCAGCTTGAGCGCCTCCGTTGGCGACAGAGTTACAA |
| | TCACCTGCAGGGCCTCTCAGAGCATCAGCAGCTATTTGAATTGGTATCAACA |
| | GAAGCCAGGAAAGGCCCCTAAGCTGCTCATCTACGCTGCCAGCTCGCTCCA |
| | ATCTGGCGTTCCTAGCAGATTTAGCGGCTCCGGCAGCGGCACAGACTTTACT |
| | CTTACCATTAGCTCCCTGCAGCCCGAGGACTTCGCTACCTACTATTGCCAGC |
| | AAAGCTACAGATTCCCTCCCACCTTTGGCCAGGGCACAAAGGTTGAGATCA |
| | AG |
| (Ab5) | SEQ ID NO: 35 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAAGAGAGCGGACCTGG |
| | CTTAGTGAAGCCCAGCGAAACCCTGTCCCTCACCTGCACCGTTTCTGGCGGA |
| | AGCATCAGCTCTCCCGACCATTACTGGGGATGGATCAGGCAGCCCCCTGGC |
| | AAGGGTTTGGAATGGATCGGCAGCATCTACGCCAGCGGCAGCACATTCTAC |
| | AACCCCTCGCTCAAAAGCAGGGTTACTATTTCTGTGGACACAAGCAAAAAT |
| | CAGTTCAGCCTGAAGCTGAGCTCTGTGACTGCCGCCGACACAGCTGTGTACT |
| | ACTGTGCCAGAGAGACAGACTACTCCAGCGGGATGGGCTACGGCATGGATG |
| | TGTGGGGACAAGGAACCACCGTTACTGTGAGCAGCGGCTCCACAAGCGGCT |
| | CAGGCAAGCCTGGCTCAGGAGAAGGAAGCACCAAGGGGGACATTCAAATG |
| | ACCCAAAGCCCCTCCAGCCTGTCCGCCAGCGTTGGCGATAGGGTTACCATTA |
| | CCTGCAGGGCCAGCCAAAGCATCAACTCCTACCTAAATTGGTATCAACAGA |
| | AGCCAGGCAAGGCCCCCAAACTACTCATTTACGCCGCCAGCAGCTTACAGA |
| | GCGGGGTTCCCTCTAGATTCTCCGGCAGCGGTTCTGGAACAGATTTCACTCT |
| | CACAATATCTTCGCTGCAGCCCGAGGATTTCGCTACCTACTATTGCCAGCAA |
| | TCCCTGGCCGACCCCTTCACATTCGGCGGAGGCACAAAGGTTGAGATCAAG |
| (Ab6) | SEQ ID NO: 36 |
| | ggtaccCCCGGgCCCATGGCTCTTCCTGTGACAGCTCTTCTGCTGCCCCTGGCCC |
| | TGCTTCTGCATGCTGCTAGACCTCAGATCACATTAAAAGAGAGCGGACCTA |
| | CACTGGTGAAGCCCACCCAAACGCTTACCCTCACCTGCACCTTTAGCGGGTT |
| | CAGCCTGGACACAGAGGGCGTTGGCGTTGGATGGATCAGGCAGCCTCCTGG |
| | CAAAGCCCTCGAATGGCTTGCCCTCATCTACTTCAACGACCAGAAGAGATA |
| | CAGCCCCTCCTTAAAATCTCGGCTCACAATCACCAAAGACACAAGCAAAAA |
| | TCAGGTTGTGCTCACCATGACCAACATGGACCCTGTGGACACCGCTGTGTAC |
| | TACTGTGCCAGAGACACCGGCTACAGCAGATGGTACTACGGGATGGACGTT |
| | TGGGGCCAAGGCACCACTGTGACCGTTTCCAGCGGCTCTACAAGCGGCAGC |
| | GGGAAACCTGGTTCTGGAGAGGGCAGCACAAAGGGCGACATCCAGATGAC |
| | GCAATCCCCCAGCTCTGTGAGCGCCTCTGTGGGAGACAGAGTTACAATCAC |
| | ATGCCGGGCCTCCCAGGGCATCAGCTCTTGGCTGGCATGGTATCAACAGAA |
| | GCCTGGCAAGGCTCCCAAGCTGCTCATCTATGCCGCCTCCTCCTTACAATCT |
| | GGAGTTCCCTCCAGGTTCAGCGGGAGCGGCTCAGGAACAGACTTCACCCTT |
| | ACCATCTCTAGCCTGCAACCCGAGGACTTCGCTACTTATTACTGTCAGCAGG |
| | CCTACGCCTACCCCATCACATTCGGCGGAGGAACAAAGGTTGAGATCAAG |
| (Ab7) | SEQ ID NO: 37 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCGAGAAATACTACTGGAGCTGGATCCGGCAGCCTCCCGGCAAAGGC |
| | TTAGAATGGATCGGCGAGATTTATCACAGCGGGCTCACCAACTACAACCCC |
| | AGCCTGAAATCTCGAGTTACAATCTCTGTGGACACAAGCAAGAATCAGTTC |
| | TCCCTGAAGCTGAGCAGCGTTACTGCCGCCGACACAGCTGTGTACTATTGCG |
| | CCAGAGTTAGATACGACAGCAGCGACAGCTATTACTACAGCTATGACTACG |
| | GCATGGATGTGTGGGGGCAGGGCACCACCGTTACTGTCTCCTCTGGATCTAC |
| | CAGCGGCAGCGGCAAGCCTGGATCTGGCGAAGGAAGCACAAAGGGCGACA |
| | TTGTGCTCACCCAGAGCCCCGACAGCCTGGCTGTGTCTTTAGGCGAAAGGG |
| | CTACCATCAACTGCAAGAGCAGCCAGAGCGTTCTGTACAGCAGCAACAACA |
| | AGAACTACCTTGCTTGGTATCAACAGAAGCCTGGCCAGCCCCCTAAGCTGCT |
| | CATCTACTGGGCCTCTAGCAGAGAGAGCGGGGTTCCCGATCGGTTTAGCGG |
| | CTCCGGCTCAGGAACCGATTTCACCCTCACTATCTCCAGCCTCCAGGCCGAG |
| | GATGTGGCTGTCTACTATTGTCAGCAGAGCTATAGCTTCCCCTGGACATTCG |
| | GCGGAGGCACCAAGGTTGAGATCAAG |
| (Ab8) | SEQ ID NO: 38 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCAGCCGCTATGTGTGGAGCTGGATCCGGCAGCCTCCTGGCAAAGGC |
| | CTTGAATGGATCGGAGAGATAGACAGCAGCGGCAAGACCAACTACAACCCC |
| | AGCCTGAAATCACGCGTTACAATCTCTGTGGACACAAGCAAGAATCAGTTC |
| | TCCCTGAAGCTGAGCAGCGTTACTGCCGCCGACACAGCTGTGTACTATTGCG |
| | CCAGAGTTAGATACGACAGCTCCGACAGCTATTACTACAGCTATGACTACG |
| | GCATGGATGTGTGGGGGCAGGGCACCACCGTTACAGTTAGCTCTGGAAGCA |
| | CCAGCGGCTCCGGCAAGCCTGGATCTGGTGAAGGAAGCACAAAGGGCGAC |
| | ATTGTGCTCACCCAGAGCCCCGACAGCCTGGCTGTGTCTTTAGGCGAAAGG |
| | GCTACCATCAACTGCAAGAGCAGCCAGAGCGTTCTGTACAGCAGCAACAAC |
| | AAGAACTACCTTGCATGGTATCAACAGAAGCCTGGCCAGCCTCCCAAGCTG |
| | CTCATCTACTGGGCCTCTAGCAGAGAGAGCGGGGTTCCCGATCGCTTTAGCG |
| | GCAGCGGTTCTGGCACCGATTTCACTCTTACAATCAGCAGCTTACAGGCCGA |
| | GGATGTGGCTGTCTACTATTGTCAGCAGAGCTATAGCTTCCCCTGGACATTC |
| | GGCGGAGGCACCAAGGTTGAGATCAAG |
| (Ab9) | SEQ ID NO: 39 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCAGCGGCTACGCTTGGAGCTGGATTAGACAGCCTCCTGGCAAAGGA |
| | CTAGAATGGATCGGAGAGATCGACCACAGAGGCTTCACCAACTACAACCCC |
| | AGCCTGAAATCCAGAGTTACAATCTCTGTGGACACAAGCAAGAATCAGTTC |
| | TCCCTGAAGCTGAGCAGCGTTACTGCCGCCGACACAGCTGTGTACTATTGCG |
| | CCAGGGTTAGATACGACAGCAGCGACAGCTATTACTACAGCTATGACTACG |
| | GCATGGATGTGTGGGGGCAGGGCACCACCGTTACGGTTAGCTCTGGATCTA |
| | CCAGCGGCAGCGGCAAGCCTGGCTCAGGAGAAGGAAGCACAAAGGGCGAC |
| | ATTGTGCTCACCCAGAGCCCCGACAGCCTGGCCGTTTCTTTAGGCGAAAGG |
| | GCTACCATCAACTGCAAGAGCAGCCAGAGCGTTCTGTACAGCAGCAACAAC |
| | AAGAACTACCTTGCATGGTATCAACAGAAGCCAGGCCAGCCTCCCAAGCTG |
| | CTCATCTACTGGGCCTCTAGCAGAGAGAGCGGGGTTCCCGATAGATTTTCGG |
| | GATCAGGCTCCGGCACCGATTTCACTCTTACGATCAGCAGCTTACAGGCCGA |
| | GGATGTGGCTGTCTACTATTGTCAGCAGAGCTATAGCTTCCCCTGGACATTC |
| | GGCGGAGGCACCAAGGTTGAGATCAAG |
| (Ab10) | SEQ ID NO: 40 |
| | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCCAGAAATACTACTGGAGCTGGATCCGGCAGCCTCCCGGCAAAGGC |
| | TTAGAATGGATCGGAGAGATAGACACCAGCGGCTTCACCAACTACAACCCC |
| | AGCCTGAAATCTAGGGTTACAATCTCTGTGGACACAAGCAAGAATCAGTTC |
| | TCCCTGAAGCTGAGCAGCGTTACTGCCGCCGACACAGCTGTGTACTATTGCG |
| | CCAGAGTTGGCAGATACAGCTACGGCTACTACATCACCGCCTTCGACATTTG |
| | GGGCCAAGGCACCACTGTGACCGTTTCCAGCGGAAGCACTAGCGGCAGCGG |
| | GAAACCTGGTTCTGGAGAGGGCTCAACCAAGGGCGACATCGTGATGACACA |
| | GAGCCCCGACTCTCTGGCTGTGTCCCTGGGAGAGAGAGCCACCATCAACTG |
| | CAAGAGCAGCCAGAGCGTTCTGTACAGCAGCAACAACAAGAACTACCTGGC |
| | ATGGTATCAACAGAAGCCTGGCCAGCCCCCTAAGCTGCTCATCTACTGGGCT |
| | TCCACCAGAGAATCAGGCGTTCCAGACAGGTTCTCCGGCTCGGGTTCAGGC |
| | ACAGACTTCACCCTTACCATCTCTTCCCTGCAGGCCGAAGATGTGGCCGTTT |
| | ACTACTGTCAGCAGCACTACAGCTTCCCTTTCACATTCGGCGGAGGCACCAA |
| | GGTTGAGATCAAG |

Exemplary nucleotide sequences encoding a binding motif, hinge, and costimulatory domain are provided in Table 5.

TABLE 5
Nucleotide sequences encoding an anti-CD20 binding motif, hinge, and
costimulatory domain

| SEQ | |
|---|---|
| ID NO | Sequence |
| 41 | ggtaccCCCGGgCCCATGGCTCTTCCTGTGACAGCTCTTCTGCTGCCCCTGGCCC |
| | TGCTTCTGCATGCTGCTAGACCTCAGCTTCAGCTCCAAGAGAGCGGACCTGG |
| | CTTAGTGAAGCCCAGCGAAACCCTGTCCCTCACCTGCACCGTTTCTGGCGGA |
| | AGCATCAGCAGCTCCAGCTATTACTGGGGATGGATCAGGCAGCCCCCTGGCA |
| | AGGGTTTAGAATGGATCGGCTCGATATATTACTCCGGCAGCACCTACTATAA |
| | CCCCAGCTTGAAGAGCCGGGTTACCATTTCTGTGGACACATCAAAGAACCAG |
| | TTCAGCCTGAAGCTGAGCTCTGTGACTGCCGCCGACACAGCTGTGTACTACT |
| | GTGCCAGAGAGACAGACTACTCCAGCGGCATGGGCTACGGCATGGATGTGT |
| | GGGGACAAGGAACCACCGTTACTGTGAGCAGCGGTTCCACCAGCGGCTCAG |
| | GCAAGCCTGGCTCAGGAGAAGGAAGCACCAAGGGGGATATACAGATGACAC |
| | AGAGCCCCTCCAGCCTGTCCGCCAGCGTTGGCGATCGTGTAACGATCACCTG |
| | CCGGGCCTCTCAGAGCATCAACTCCTACCTCAATTGGTATCAACAGAAGCCA |
| | GGCAAGGCCCCCAAATTACTCATCTACGCCGCCAGCAGCTTACAGAGCGGG |
| | GTTCCCTCTAGATTCTCCGGCTCCGGTTCTGGAACAGATTTCACCCTCACTAT |
| | CTCCAGCTTGCAGCCCGAGGATTTCGCCACTTATTACTGTCAGCAGAGCCTG |
| | GCCGACCCCTTCACATTCGGCGGAGGCACAAAGGTTGAGATCAAGGCAGCT |
| | GCTTTCGTGCCTGTGTTCCTGCCTGCTAAGCCCACCACCACTCCTGCTCCAAG |
| | ACCTCCTACCCCCGCTCCTACAATCGCCAGCCAACCTCTGAGCCTGAGACCG |
| | GAGGCATGCAGACCTGCGGCAGGGGGAGCAGTTCACACAAGAGGCTTGGAC |
| | TTCGCTTGCGACATCTACATCTGGGCCCCTCTGGCCGGCACATGCGGAGTTCT |
| | TCTTCTTAGCCTGGTGATCACCCTGTACTGCAACCACAGAAACAGATTCAGC |
| | GTTGTGAAGAGAGGCCGGAAGAAGCTGCTGTACATCTTCAAGCAGCCCTTCA |
| | TGAGACCTGTGCAGACCACACAGGAGGAAGACGGCTGCAGCTGTAGATTCC |
| | CCGAGGAAGAGGAGGGCGGCTGTGAGCTGAGAGTTAAGTTCAGCAGGAGCG |
| | CCGACGCCCCTGCCTACCAGCAAGGACAGAATCAACTGTACAACGAGCTGA |
| | ACCTGGGCAGACGGGAGGAATACGATGTGCTGGACAAGAGGAGAGGCAGA |
| | GACCCCGAGATGGGCGGCAAACCTAGAAGAAAGAACCCCCAGGAGGGCCTG |
| | TATAACGAGCTCCAGAAGGACAAGATGGCCGAGGCCTACAGCGAGATCGGC |
| | ATGAAGGGCGAAAGAAGAAGAGGCAAGGGCCACGACGGCCTCTACCAGGG |
| | CTTAAGCACAGCTACAAAGGACACCTACGACGCCCTGCACATGCAGGCCCTG |
| | CCCCCTAGATGATTAATTAAatcgat |
| 42 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAAGAGAGCGGACCTGG |
| | CTTAGTGAAGCCCAGCGAAACCCTGTCCCTCACCTGCACCGTTTCTGGCGGA |
| | AGCATCAGCTCTCCCGACCATTACTGGGGATGGATCAGGCAGCCCCCTGGCA |
| | AGGGTTTGGAATGGATCGGCAGCATCTACGCCAGCGGCAGCACATTCTACAA |
| | CCCCTCGCTCAAAAGCAGGGTTACTATTTCTGTGGACACAAGCAAAAATCAG |
| | TTCAGCCTGAAGCTGAGCTCTGTGACTGCCGCCGACACAGCTGTGTACTACT |
| | GTGCCAGAGAGACAGACTACTCCAGCGGGATGGGCTACGGCATGGATGTGT |
| | GGGGACAAGGAACCACCGTTACTGTGAGCAGCGGCTCCACAAGCGGCTCAG |
| | GCAAGCCTGGCTCAGGAGAAGGAAGCACCAAGGGGGACATTCAAATGACCC |
| | AAAGCCCCTCCAGCCTGTCCGCCAGCGTTGGCGATAGGGTTACCATTACCTG |
| | CAGGGCCAGCCAAAGCATCAACTCCTACCTAAATTGGTATCAACAGAAGCC |
| | AGGCAAGGCCCCCAAACTACTCATTTACGCCGCCAGCAGCTTACAGAGCGG |
| | GGTTCCCTCTAGATTCTCCGGCAGCGGTTCTGGAACAGATTTCACTCTCACAA |
| | TATCTTCGCTGCAGCCCGAGGATTTCGCTACCTACTATTGCCAGCAATCCCTG |
| | GCCGACCCCTTCACATTCGGCGGAGGCACAAAGGTTGAGATCAAGGCAGCT |
| | GCTTTCGTGCCTGTGTTCCTGCCTGCTAAGCCCACCACCACTCCTGCTCCAAG |
| | ACCTCCTACCCCCGCTCCTACAATCGCCAGCCAACCTCTGAGCCTGAGACCG |
| | GAGGCATGCAGACCTGCGGCAGGGGGAGCAGTTCACACAAGAGGCTTGGAC |
| | TTCGCTTGCGACATCTACATCTGGGCCCCTCTGGCCGGCACATGCGGAGTTCT |
| | TCTTCTTAGCCTGGTGATCACCCTGTACTGCAACCACAGAAACAGATTCAGC |
| | GTTGTGAAGAGAGGCCGGAAGAAGCTGCTGTACATCTTCAAGCAGCCCTTCA |
| | TGAGACCTGTGCAGACCACACAGGAGGAAGACGGCTGCAGCTGTAGATTCC |
| | CCGAGGAAGAGGAGGGCGGCTGTGAGCTGAGAGTTAAGTTCAGCAGGAGCG |
| | CCGACGCCCCTGCCTACCAGCAAGGACAGAATCAACTGTACAACGAGCTGA |
| | ACCTGGGCAGACGGGAGGAATACGATGTGCTGGACAAGAGGAGAGGCAGA |
| | GACCCCGAGATGGGCGGCAAACCTAGAAGAAAGAACCCCCAGGAGGGCCTG |
| | TATAACGAGCTCCAGAAGGACAAGATGGCCGAGGCCTACAGCGAGATCGGC |
| | ATGAAGGGCGAAAGAAGAAGAGGCAAGGGCCACGACGGCCTCTACCAGGG |
| | CTTAAGCACAGCTACAAAGGACACCTACGACGCCCTGCACATGCAGGCCCTG |
| | CCCCCTAGATGATTAATTAAatcgat |
| 43 | ggtaccCCCGGgCCCATGGCTCTTCCTGTGACAGCTCTTCTGCTGCCCCTGGCCC |
| | TGCTTCTGCATGCTGCTAGACCTCAGATCACATTAAAAGAGAGCGGACCTAC |
| | ACTGGTGAAGCCCACCCAAACGCTTACCCTCACCTGCACCTTTAGCGGGTTC |
| | AGCCTGGACACAGAGGGCGTTGGCGTTGGATGGATCAGGCAGCCTCCTGGC |
| | AAAGCCCTCGAATGGCTTGCCCTCATCTACTTCAACGACCAGAAGAGATACA |
| | GCCCCTCCTTAAAATCTCGGCTCACAATCACCAAAGACACAAGCAAAAATCA |
| | GGTTGTGCTCACCATGACCAACATGGACCCTGTGGACACCGCTGTGTACTAC |
| | TGTGCCAGAGACACCGGCTACAGCAGATGGTACTACGGGATGGACGTTTGG |
| | GGCCAAGGCACCACTGTGACCGTTTCCAGCGGCTCTACAAGCGGCAGCGGG |
| | AAACCTGGTTCTGGAGAGGGCAGCACAAAGGGCGACATCCAGATGACGCAA |
| | TCCCCCAGCTCTGTGAGCGCCTCTGTGGGAGACAGAGTTACAATCACATGCC |
| | GGGCCTCCCAGGGCATCAGCTCTTGGCTGGCATGGTATCAACAGAAGCCTGG |
| | CAAGGCTCCCAAGCTGCTCATCTATGCCGCCTCCTCCTTACAATCTGGAGTTC |
| | CCTCCAGGTTCAGCGGGAGCGGCTCAGGAACAGACTTCACCCTTACCATCTC |
| | TAGCCTGCAACCCGAGGACTTCGCTACTTATTACTGTCAGCAGGCCTACGCC |
| | TACCCCATCACATTCGGCGGAGGAACAAAGGTTGAGATCAAGGCAGCTGCTT |
| | TCGTGCCTGTGTTCCTGCCTGCTAAGCCCACCACCACTCCTGCTCCAAGACCT |
| | CCTACCCCCGCTCCTACAATCGCCAGCCAACCTCTGAGCCTGAGACCGGAGG |
| | CATGCAGACCTGCGGCAGGGGGAGCAGTTCACACAAGAGGCTTGGACTTCG |
| | CTTGCGACATCTACATCTGGGCCCCTCTGGCCGGCACATGCGGAGTTCTTCTT |
| | CTTAGCCTGGTGATCACCCTGTACTGCAACCACAGAAACAGATTCAGCGTTG |
| | TGAAGAGAGGCCGGAAGAAGCTGCTGTACATCTTCAAGCAGCCCTTCATGA |
| | GACCTGTGCAGACCACACAGGAGGAAGACGGCTGCAGCTGTAGATTCCCCG |
| | AGGAAGAGGAGGGCGGCTGTGAGCTGAGAGTTAAGTTCAGCAGGAGCGCCG |
| | ACGCCCCTGCCTACCAGCAAGGACAGAATCAACTGTACAACGAGCTGAACC |
| | TGGGCAGACGGGAGGAATACGATGTGCTGGACAAGAGGAGAGGCAGAGAC |
| | CCCGAGATGGGCGGCAAACCTAGAAGAAAGAACCCCCAGGAGGGCCTGTAT |
| | AACGAGCTCCAGAAGGACAAGATGGCCGAGGCCTACAGCGAGATCGGCATG |
| | AAGGGCGAAAGAAGAAGAGGCAAGGGCCACGACGGCCTCTACCAGGGCTTA |
| | AGCACAGCTACAAAGGACACCTACGACGCCCTGCACATGCAGGCCCTGCCC |
| | CCTAGATGATTAATTAAatcgat |
| 44 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCCAGAAATACTACTGGAGCTGGATCCGGCAGCCTCCCGGCAAAGGCT |
| | TAGAATGGATCGGAGAGATAGACACCAGCGGCTTCACCAACTACAACCCCA |
| | GCCTGAAATCTAGGGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGAGTTGGCAGATACAGCTACGGCTACTACATCACCGCCTTCGACATTTGGG |
| | GCCAAGGCACCACTGTGACCGTTTCCAGCGGAAGCACTAGCGGCAGCGGGA |
| | AACCTGGTTCTGGAGAGGGCTCAACCAAGGGCGACATCGTGATGACACAGA |
| | GCCCCGACTCTCTGGCTGTGTCCCTGGGAGAGAGAGCCACCATCAACTGCAA |
| | GAGCAGCCAGAGCGTTCTGTACAGCAGCAACAACAAGAACTACCTGGCATG |
| | GTATCAACAGAAGCCTGGCCAGCCCCCTAAGCTGCTCATCTACTGGGCTTCC |
| | ACCAGAGAATCAGGCGTTCCAGACAGGTTCTCCGGCTCGGGTTCAGGCACAG |
| | ACTTCACCCTTACCATCTCTTCCCTGCAGGCCGAAGATGTGGCCGTTTACTAC |
| | TGTCAGCAGCACTACAGCTTCCCTTTCACATTCGGCGGAGGCACCAAGGTTG |
| | AGATCAAGGCAGCTGCTTTCGTGCCTGTGTTCCTGCCTGCTAAGCCCACCAC |
| | CACTCCTGCTCCAAGACCTCCTACCCCCGCTCCTACAATCGCCAGCCAACCTC |
| | TGAGCCTGAGACCGGAGGCATGCAGACCTGCGGCAGGGGGAGCAGTTCACA |
| | CAAGAGGCTTGGACTTCGCTTGCGACATCTACATCTGGGCCCCTCTGGCCGG |
| | CACATGCGGAGTTCTTCTTCTTAGCCTGGTGATCACCCTGTACTGCAACCACA |
| | GAAACAGATTCAGCGTTGTGAAGAGAGGCCGGAAGAAGCTGCTGTACATCT |
| | TCAAGCAGCCCTTCATGAGACCTGTGCAGACCACACAGGAGGAAGACGGCT |
| | GCAGCTGTAGATTCCCCGAGGAAGAGGAGGGCGGCTGTGAGCTGAGAGTTA |
| | AGTTCAGCAGGAGCGCCGACGCCCCTGCCTACCAGCAAGGACAGAATCAAC |
| | TGTACAACGAGCTGAACCTGGGCAGACGGGAGGAATACGATGTGCTGGACA |
| | AGAGGAGAGGCAGAGACCCCGAGATGGGCGGCAAACCTAGAAGAAAGAAC |
| | CCCCAGGAGGGCCTGTATAACGAGCTCCAGAAGGACAAGATGGCCGAGGCC |
| | TACAGCGAGATCGGCATGAAGGGCGAAAGAAGAAGAGGCAAGGGCCACGA |
| | CGGCCTCTACCAGGGCTTAAGCACAGCTACAAAGGACACCTACGACGCCCTG |
| | CACATGCAGGCCCTGCCCCCTAGATGATTAATTAAatcgat |
| 45 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCGAGAAATACTACTGGAGCTGGATCCGGCAGCCTCCCGGCAAAGGCT |
| | TAGAATGGATCGGCGAGATTTATCACAGCGGGCTCACCAACTACAACCCCAG |
| | CCTGAAATCTCGAGTTACAATCTCTGTGGACACAAGCAAGAATCAGTTCTCC |
| | CTGAAGCTGAGCAGCGTTACTGCCGCCGACACAGCTGTGTACTATTGCGCCA |
| | GAGTTAGATACGACAGCAGCGACAGCTATTACTACAGCTATGACTACGGCAT |
| | GGATGTGTGGGGGCAGGGCACCACCGTTACTGTCTCCTCTGGATCTACCAGC |
| | GGCAGCGGCAAGCCTGGATCTGGCGAAGGAAGCACAAAGGGCGACATTGTG |
| | CTCACCCAGAGCCCCGACAGCCTGGCTGTGTCTTTAGGCGAAAGGGCTACCA |
| | TCAACTGCAAGAGCAGCCAGAGCGTTCTGTACAGCAGCAACAACAAGAACT |
| | ACCTTGCTTGGTATCAACAGAAGCCTGGCCAGCCCCCTAAGCTGCTCATCTA |
| | CTGGGCCTCTAGCAGAGAGAGCGGGGTTCCCGATCGGTTTAGCGGCTCCGGC |
| | TCAGGAACCGATTTCACCCTCACTATCTCCAGCCTCCAGGCCGAGGATGTGG |
| | CTGTCTACTATTGTCAGCAGAGCTATAGCTTCCCCTGGACATTCGGCGGAGG |
| | CACCAAGGTTGAGATCAAGGCAGCTGCTTTCGTGCCTGTGTTCCTGCCTGCT |
| | AAGCCCACCACCACTCCTGCTCCAAGACCTCCTACCCCCGCTCCTACAATCG |
| | CCAGCCAACCTCTGAGCCTGAGACCGGAGGCATGCAGACCTGCGGCAGGGG |
| | GAGCAGTTCACACAAGAGGCTTGGACTTCGCTTGCGACATCTACATCTGGGC |
| | CCCTCTGGCCGGCACATGCGGAGTTCTTCTTCTTAGCCTGGTGATCACCCTGT |
| | ACTGCAACCACAGAAACAGATTCAGCGTTGTGAAGAGAGGCCGGAAGAAGC |
| | TGCTGTACATCTTCAAGCAGCCCTTCATGAGACCTGTGCAGACCACACAGGA |
| | GGAAGACGGCTGCAGCTGTAGATTCCCCGAGGAAGAGGAGGGCGGCTGTGA |
| | GCTGAGAGTTAAGTTCAGCAGGAGCGCCGACGCCCCTGCCTACCAGCAAGG |
| | ACAGAATCAACTGTACAACGAGCTGAACCTGGGCAGACGGGAGGAATACGA |
| | TGTGCTGGACAAGAGGAGAGGCAGAGACCCCGAGATGGGCGGCAAACCTAG |
| | AAGAAAGAACCCCCAGGAGGGCCTGTATAACGAGCTCCAGAAGGACAAGAT |
| | GGCCGAGGCCTACAGCGAGATCGGCATGAAGGGCGAAAGAAGAAGAGGCA |
| | AGGGCCACGACGGCCTCTACCAGGGCTTAAGCACAGCTACAAAGGACACCT |
| | ACGACGCCCTGCACATGCAGGCCCTGCCCCCTAGATGATTAATTAAatcgat |
| 46 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCAGCCGCTATGTGTGGAGCTGGATCCGGCAGCCTCCTGGCAAAGGCC |
| | TTGAATGGATCGGAGAGATAGACAGCAGCGGCAAGACCAACTACAACCCCA |
| | GCCTGAAATCACGCGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGAGTTAGATACGACAGCTCCGACAGCTATTACTACAGCTATGACTACGGCA |
| | TGGATGTGTGGGGGCAGGGCACCACCGTTACAGTTAGCTCTGGAAGCACCA |
| | GCGGCTCCGGCAAGCCTGGATCTGGTGAAGGAAGCACAAAGGGCGACATTG |
| | TGCTCACCCAGAGCCCCGACAGCCTGGCTGTGTCTTTAGGCGAAAGGGCTAC |
| | CATCAACTGCAAGAGCAGCCAGAGCGTTCTGTACAGCAGCAACAACAAGAA |
| | CTACCTTGCATGGTATCAACAGAAGCCTGGCCAGCCTCCCAAGCTGCTCATC |
| | TACTGGGCCTCTAGCAGAGAGAGCGGGGTTCCCGATCGCTTTAGCGGCAGCG |
| | GTTCTGGCACCGATTTCACTCTTACAATCAGCAGCTTACAGGCCGAGGATGT |
| | GGCTGTCTACTATTGTCAGCAGAGCTATAGCTTCCCCTGGACATTCGGCGGA |
| | GGCACCAAGGTTGAGATCAAGGCAGCTGCTTTCGTGCCTGTGTTCCTGCCTG |
| | CTAAGCCCACCACCACTCCTGCTCCAAGACCTCCTACCCCCGCTCCTACAATC |
| | GCCAGCCAACCTCTGAGCCTGAGACCGGAGGCATGCAGACCTGCGGCAGGG |
| | GGAGCAGTTCACACAAGAGGCTTGGACTTCGCTTGCGACATCTACATCTGGG |
| | CCCCTCTGGCCGGCACATGCGGAGTTCTTCTTCTTAGCCTGGTGATCACCCTG |
| | TACTGCAACCACAGAAACAGATTCAGCGTTGTGAAGAGAGGCCGGAAGAAG |
| | CTGCTGTACATCTTCAAGCAGCCCTTCATGAGACCTGTGCAGACCACACAGG |
| | AGGAAGACGGCTGCAGCTGTAGATTCCCCGAGGAAGAGGAGGGCGGCTGTG |
| | AGCTGAGAGTTAAGTTCAGCAGGAGCGCCGACGCCCCTGCCTACCAGCAAG |
| | GACAGAATCAACTGTACAACGAGCTGAACCTGGGCAGACGGGAGGAATACG |
| | ATGTGCTGGACAAGAGGAGAGGCAGAGACCCCGAGATGGGCGGCAAACCTA |
| | GAAGAAAGAACCCCCAGGAGGGCCTGTATAACGAGCTCCAGAAGGACAAGA |
| | TGGCCGAGGCCTACAGCGAGATCGGCATGAAGGGCGAAAGAAGAAGAGGC |
| | AAGGGCCACGACGGCCTCTACCAGGGCTTAAGCACAGCTACAAAGGACACC |
| | TACGACGCCCTGCACATGCAGGCCCTGCCCCCTAGATGATTAATTAAatcgat |
| 47 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCAGCGGCTACGCTTGGAGCTGGATTAGACAGCCTCCTGGCAAAGGAC |
| | TAGAATGGATCGGAGAGATCGACCACAGAGGCTTCACCAACTACAACCCCA |
| | GCCTGAAATCCAGAGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGGGTTAGATACGACAGCAGCGACAGCTATTACTACAGCTATGACTACGGC |
| | ATGGATGTGTGGGGGCAGGGCACCACCGTTACGGTTAGCTCTGGATCTACCA |
| | GCGGCAGCGGCAAGCCTGGCTCAGGAGAAGGAAGCACAAAGGGCGACATTG |
| | TGCTCACCCAGAGCCCCGACAGCCTGGCCGTTTCTTTAGGCGAAAGGGCTAC |
| | CATCAACTGCAAGAGCAGCCAGAGCGTTCTGTACAGCAGCAACAACAAGAA |
| | CTACCTTGCATGGTATCAACAGAAGCCAGGCCAGCCTCCCAAGCTGCTCATC |
| | TACTGGGCCTCTAGCAGAGAGAGCGGGGTTCCCGATAGATTTTCGGGATCAG |
| | GCTCCGGCACCGATTTCACTCTTACGATCAGCAGCTTACAGGCCGAGGATGT |
| | GGCTGTCTACTATTGTCAGCAGAGCTATAGCTTCCCCTGGACATTCGGCGGA |
| | GGCACCAAGGTTGAGATCAAGGCAGCTGCTTTCGTGCCTGTGTTCCTGCCTG |
| | CTAAGCCCACCACCACTCCTGCTCCAAGACCTCCTACCCCCGCTCCTACAATC |
| | GCCAGCCAACCTCTGAGCCTGAGACCGGAGGCATGCAGACCTGCGGCAGGG |
| | GGAGCAGTTCACACAAGAGGCTTGGACTTCGCTTGCGACATCTACATCTGGG |
| | CCCCTCTGGCCGGCACATGCGGAGTTCTTCTTCTTAGCCTGGTGATCACCCTG |
| | TACTGCAACCACAGAAACAGATTCAGCGTTGTGAAGAGAGGCCGGAAGAAG |
| | CTGCTGTACATCTTCAAGCAGCCCTTCATGAGACCTGTGCAGACCACACAGG |
| | AGGAAGACGGCTGCAGCTGTAGATTCCCCGAGGAAGAGGAGGGCGGCTGTG |
| | AGCTGAGAGTTAAGTTCAGCAGGAGCGCCGACGCCCCTGCCTACCAGCAAG |
| | GACAGAATCAACTGTACAACGAGCTGAACCTGGGCAGACGGGAGGAATACG |
| | ATGTGCTGGACAAGAGGAGAGGCAGAGACCCCGAGATGGGCGGCAAACCTA |
| | GAAGAAAGAACCCCCAGGAGGGCCTGTATAACGAGCTCCAGAAGGACAAGA |
| | TGGCCGAGGCCTACAGCGAGATCGGCATGAAGGGCGAAAGAAGAAGAGGC |
| | AAGGGCCACGACGGCCTCTACCAGGGCTTAAGCACAGCTACAAAGGACACC |
| | TACGACGCCCTGCACATGCAGGCCCTGCCCCCTAGATGATTAATTAAatcgat |
| 48 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCAGCGGCTATTACTGGAGCTGGATCCGGCAGCCTCCTGGAAAAGGAT |
| | TAGAATGGATCGGCGAGATAGACCACAGCGGGAGCACAAACTACAACCCCA |
| | GCCTGAAATCGCGGGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGAGGCGGAGGCTCCTGGTACAGCAACTGGTTCGATCCTTGGGGCCAAGGC |
| | ACCATGGTGACCGTTTCCAGCGGCTCTACAAGCGGCAGCGGGAAACCTGGTT |
| | CTGGAGAGGGCAGCACAAAGGGCGACATCCAGATGACACAGAGCCCCAGCA |
| | CCCTTAGCGCCTCTGTGGGAGATAGGGTTACCATTACCTGCAGGGCTTCCCA |
| | GAGCATCAGCAGCTGGCTGGCATGGTATCAACAGAAGCCTGGCAAGGCTCC |
| | CAAGCTGCTCATCTATGACGCCTCCAGCCTGGAAAGCGGGGTTCCCTCCAGA |
| | TTTAGCGGCTCAGGCTCCGGAACAGAGTTCACCCTTACCATCTCTAGCCTGC |
| | AACCCGACGACTTCGCTACTTATTACTGTCAACAAGACAGAAGCTTGCCCCC |
| | CACATTCGGCGGAGGGACCAAGGTTGAGATCAAGGCAGCTGCTTTCGTGCCT |
| | GTGTTCCTGCCTGCTAAGCCCACCACCACTCCTGCTCCAAGACCTCCTACCCC |
| | CGCTCCTACAATCGCCAGCCAACCTCTGAGCCTGAGACCGGAGGCATGCAGA |
| | CCTGCGGCAGGGGGAGCAGTTCACACAAGAGGCTTGGACTTCGCTTGCGAC |
| | ATCTACATCTGGGCCCCTCTGGCCGGCACATGCGGAGTTCTTCTTCTTAGCCT |
| | GGTGATCACCCTGTACTGCAACCACAGAAACAGATTCAGCGTTGTGAAGAG |
| | AGGCCGGAAGAAGCTGCTGTACATCTTCAAGCAGCCCTTCATGAGACCTGTG |
| | CAGACCACACAGGAGGAAGACGGCTGCAGCTGTAGATTCCCCGAGGAAGAG |
| | GAGGGCGGCTGTGAGCTGAGAGTTAAGTTCAGCAGGAGCGCCGACGCCCCT |
| | GCCTACCAGCAAGGACAGAATCAACTGTACAACGAGCTGAACCTGGGCAGA |
| | CGGGAGGAATACGATGTGCTGGACAAGAGGAGAGGCAGAGACCCCGAGAT |
| | GGGCGGCAAACCTAGAAGAAAGAACCCCCAGGAGGGCCTGTATAACGAGCT |
| | CCAGAAGGACAAGATGGCCGAGGCCTACAGCGAGATCGGCATGAAGGGCGA |
| | AAGAAGAAGAGGCAAGGGCCACGACGGCCTCTACCAGGGCTTAAGCACAGC |
| | TACAAAGGACACCTACGACGCCCTGCACATGCAGGCCCTGCCCCCTAGATGA |
| | TTAATTAAatcgat |
| 49 | ggtaccCCCGGgCCCATGGCTCTTCCTGTGACAGCTCTTCTGCTGCCCCTGGCCC |
| | TGCTTCTGCATGCTGCTAGACCTCAGGTTCAGCTTGTGCAGAGCGGAGCTGA |
| | AGTTAAGAAGCCTGGCGCCTCTGTGAAGGTTAGCTGCAAGGCCAGCGGCTAC |
| | ACATTCAAGGAATATGGCATCTCCTGGGTTAGGCAGGCTCCCGGCCAAGGCT |
| | TAGAATGGATGGGCTGGATCTCCGCCTACTCCGGCCACACCTACTACGCCCA |
| | GAAGCTTCAGGGCAGGGTTACCATGACCACCGACACCAGCACCTCTACCGCC |
| | TATATGGAGCTGAGGAGCCTGAGATCGGACGACACAGCTGTGTATTACTGCG |
| | CCAGAGGCCCCCACTACGACGACTGGTCTGGATTTATCATCTGGTTCGACCC |
| | CTGGGGGCAGGGCACCCTGGTCACAGTTTCTTCTGGCTCCACCAGCGGAAGC |
| | GGCAAGCCAGGCTCAGGCGAAGGATCTACAAAAGGCGACATCCAAATGACA |
| | CAGAGCCCCAGCAGCTTGAGCGCCTCCGTTGGCGACAGAGTTACAATCACCT |
| | GCAGGGCCTCTCAGAGCATCAGCAGCTATTTGAATTGGTATCAACAGAAGCC |
| | AGGAAAGGCCCCTAAGCTGCTCATCTACGCTGCCAGCTCGCTCCAATCTGGC |
| | GTTCCTAGCAGATTTAGCGGCTCCGGCAGCGGCACAGACTTTACTCTTACCA |
| | TTAGCTCCCTGCAGCCCGAGGACTTCGCTACCTACTATTGCCAGCAAAGCTA |
| | CAGATTCCCTCCCACCTTTGGCCAGGGCACAAAGGTTGAGATCAAGGCAGCT |
| | GCTTTCGTGCCTGTGTTCCTGCCTGCTAAGCCCACCACCACTCCTGCTCCAAG |
| | ACCTCCTACCCCCGCTCCTACAATCGCCAGCCAACCTCTGAGCCTGAGACCG |
| | GAGGCATGCAGACCTGCGGCAGGGGGAGCAGTTCACACAAGAGGCTTGGAC |
| | TTCGCTTGCGACATCTACATCTGGGCCCCTCTGGCCGGCACATGCGGAGTTCT |
| | TCTTCTTAGCCTGGTGATCACCCTGTACTGCAACCACAGAAACAGATTCAGC |
| | GTTGTGAAGAGAGGCCGGAAGAAGCTGCTGTACATCTTCAAGCAGCCCTTCA |
| | TGAGACCTGTGCAGACCACACAGGAGGAAGACGGCTGCAGCTGTAGATTCC |
| | CCGAGGAAGAGGAGGGCGGCTGTGAGCTGAGAGTTAAGTTCAGCAGGAGCG |
| | CCGACGCCCCTGCCTACCAGCAAGGACAGAATCAACTGTACAACGAGCTGA |
| | ACCTGGGCAGACGGGAGGAATACGATGTGCTGGACAAGAGGAGAGGCAGA |
| | GACCCCGAGATGGGCGGCAAACCTAGAAGAAAGAACCCCCAGGAGGGCCTG |
| | TATAACGAGCTCCAGAAGGACAAGATGGCCGAGGCCTACAGCGAGATCGGC |
| | ATGAAGGGCGAAAGAAGAAGAGGCAAGGGCCACGACGGCCTCTACCAGGG |
| | CTTAAGCACAGCTACAAAGGACACCTACGACGCCCTGCACATGCAGGCCCTG |
| | CCCCCTAGATGATTAATTAAatcgat |
| 50 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCAGCGGCATCCACTGGAACTGGATCCGGCAGCCTCCTGGCAAAGGCC |
| | TTGAATGGATCGGCGATATCGACACCAGCGGCTCCACCAACTACAACCCCAG |
| | CCTGAAATCGAGGGTTACAATCTCTGTGGACACAAGCAAGAATCAGTTCTCC |
| | CTGAAGCTGAGCAGCGTTACTGCCGCCGACACAGCTGTGTACTATTGCGCCA |
| | GACTGGGCCAGGAAAGCGCTACCTACCTTGGCATGGATGTGTGGGGGCAGG |
| | GCACCACCGTTACTGTTAGCTCTGGCTCAACAAGCGGCAGCGGCAAGCCTGG |
| | CTCAGGAGAAGGAAGCACAAAGGGCGACATTGTAATGACTCAGAGCCCCGA |
| | CAGCCTGGCCGTTAGCTTAGGCGAAAGGGCTACAATCAATTGCAAGAGCAG |
| | CCAGAGCGTTCTGTACAGCAGCAACAACAAGAACTACCTCGCATGGTATCAA |
| | CAGAAGCCAGGCCAGCCTCCCAAGCTGCTCATCTACTGGGCTTCCACCAGAG |
| | AGAGCGGGGTTCCCGATAGATTCTCCGGCTCCGGTTCTGGAACAGATTTCAC |
| | GCTCACAATCAGCAGCTTACAGGCCGAGGATGTGGCTGTCTACTATTGTCAG |
| | CAGTTGTACACCTACCCCTTCACATTCGGCGGAGGCACCAAGGTTGAGATCA |
| | AGGCAGCTGCTTTCGTGCCTGTGTTCCTGCCTGCTAAGCCCACCACCACTCCT |
| | GCTCCAAGACCTCCTACCCCCGCTCCTACAATCGCCAGCCAACCTCTGAGCC |
| | TGAGACCGGAGGCATGCAGACCTGCGGCAGGGGGAGCAGTTCACACAAGAG |
| | GCTTGGACTTCGCTTGCGACATCTACATCTGGGCCCCTCTGGCCGGCACATG |
| | CGGAGTTCTTCTTCTTAGCCTGGTGATCACCCTGTACTGCAACCACAGAAAC |
| | AGATTCAGCGTTGTGAAGAGAGGCCGGAAGAAGCTGCTGTACATCTTCAAG |
| | CAGCCCTTCATGAGACCTGTGCAGACCACACAGGAGGAAGACGGCTGCAGC |
| | TGTAGATTCCCCGAGGAAGAGGAGGGCGGCTGTGAGCTGAGAGTTAAGTTC |
| | AGCAGGAGCGCCGACGCCCCTGCCTACCAGCAAGGACAGAATCAACTGTAC |
| | AACGAGCTGAACCTGGGCAGACGGGAGGAATACGATGTGCTGGACAAGAGG |
| | AGAGGCAGAGACCCCGAGATGGGCGGCAAACCTAGAAGAAAGAACCCCCA |
| | GGAGGGCCTGTATAACGAGCTCCAGAAGGACAAGATGGCCGAGGCCTACAG |
| | CGAGATCGGCATGAAGGGCGAAAGAAGAAGAGGCAAGGGCCACGACGGCC |
| | TCTACCAGGGCTTAAGCACAGCTACAAAGGACACCTACGACGCCCTGCACAT |
| | GCAGGCCCTGCCCCCTAGATGATTAATTAAatcgat |
| 51 | ggtaccCCCGGgCCCATGGCTCTTCCTGTGACAGCTCTTCTGCTGCCCCTGGCCC |
| | TGCTTCTGCATGCTGCTAGACCTCAGCTTCAGCTCCAAGAGAGCGGACCTGG |
| | CTTAGTGAAGCCCAGCGAAACCCTGTCCCTCACCTGCACCGTTTCTGGCGGA |
| | AGCATCAGCAGCTCCAGCTATTACTGGGGATGGATCAGGCAGCCCCCTGGCA |
| | AGGGTTTAGAATGGATCGGCTCGATATATTACTCCGGCAGCACCTACTATAA |
| | CCCCAGCTTGAAGAGCCGGGTTACCATTTCTGTGGACACATCAAAGAACCAG |
| | TTCAGCCTGAAGCTGAGCTCTGTGACTGCCGCCGACACAGCTGTGTACTACT |
| | GTGCCAGAGAGACAGACTACTCCAGCGGCATGGGCTACGGCATGGATGTGT |
| | GGGGACAAGGAACCACCGTTACTGTGAGCAGCGGTTCCACCAGCGGCTCAG |
| | GCAAGCCTGGCTCAGGAGAAGGAAGCACCAAGGGGGATATACAGATGACAC |
| | AGAGCCCCTCCAGCCTGTCCGCCAGCGTTGGCGATCGTGTAACGATCACCTG |
| | CCGGGCCTCTCAGAGCATCAACTCCTACCTCAATTGGTATCAACAGAAGCCA |
| | GGCAAGGCCCCCAAATTACTCATCTACGCCGCCAGCAGCTTACAGAGCGGG |
| | GTTCCCTCTAGATTCTCCGGCTCCGGTTCTGGAACAGATTTCACCCTCACTAT |
| | CTCCAGCTTGCAGCCCGAGGATTTCGCCACTTATTACTGTCAGCAGAGCCTG |
| | GCCGACCCCTTCACATTCGGCGGAGGCACAAAGGTTGAGATCAAGGCTGCTG |
| | CATTGGATAATGAAAAATCGAACGGCACAATCATTCATGTGAAGGGCAAAC |
| | ACCTGTGTCCCAGCCCCTTGTTCCCAGGACCTAGCAAGCCTTTTTGGGTTCTC |
| | GTGGTGGTGGGCGGCGTTCTGGCTTGCTACTCTCTACTTGTAACTGTCGCATT |
| | TATTATATTCTGGGTTAGATTCAGCGTTGTGAAGAGAGGCCGGAAGAAGCTG |
| | CTGTACATCTTCAAGCAGCCCTTCATGAGACCTGTGCAGACCACACAGGAGG |
| | AAGACGGCTGCAGCTGTAGATTCCCCGAGGAAGAGGAGGGCGGCTGTGAGC |
| | TGAGAGTTAAGTTCAGCAGGAGCGCCGACGCCCCTGCCTACCAGCAAGGAC |
| | AGAATCAACTGTACAACGAGCTGAACCTGGGCAGACGGGAGGAATACGATG |
| | TGCTGGACAAGAGGAGAGGCAGAGACCCCGAGATGGGCGGCAAACCTAGA |
| | AGAAAGAACCCCCAGGAGGGCCTGTATAACGAGCTCCAGAAGGACAAGATG |
| | GCCGAGGCCTACAGCGAGATCGGCATGAAGGGCGAAAGAAGAAGAGGCAA |
| | GGGCCACGACGGCCTCTACCAGGGCTTAAGCACAGCTACAAAGGACACCTA |
| | CGACGCCCTGCACATGCAGGCCCTGCCCCCTAGATGATTAATTAAatcgat |
| 52 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAAGAGAGCGGACCTGG |
| | CTTAGTGAAGCCCAGCGAAACCCTGTCCCTCACCTGCACCGTTTCTGGCGGA |
| | AGCATCAGCTCTCCCGACCATTACTGGGGATGGATCAGGCAGCCCCCTGGCA |
| | AGGGTTTGGAATGGATCGGCAGCATCTACGCCAGCGGCAGCACATTCTACAA |
| | CCCCTCGCTCAAAAGCAGGGTTACTATTTCTGTGGACACAAGCAAAAATCAG |
| | TTCAGCCTGAAGCTGAGCTCTGTGACTGCCGCCGACACAGCTGTGTACTACT |
| | GTGCCAGAGAGACAGACTACTCCAGCGGGATGGGCTACGGCATGGATGTGT |
| | GGGGACAAGGAACCACCGTTACTGTGAGCAGCGGCTCCACAAGCGGCTCAG |
| | GCAAGCCTGGCTCAGGAGAAGGAAGCACCAAGGGGGACATTCAAATGACCC |
| | AAAGCCCCTCCAGCCTGTCCGCCAGCGTTGGCGATAGGGTTACCATTACCTG |
| | CAGGGCCAGCCAAAGCATCAACTCCTACCTAAATTGGTATCAACAGAAGCC |
| | AGGCAAGGCCCCCAAACTACTCATTTACGCCGCCAGCAGCTTACAGAGCGG |
| | GGTTCCCTCTAGATTCTCCGGCAGCGGTTCTGGAACAGATTTCACTCTCACAA |
| | TATCTTCGCTGCAGCCCGAGGATTTCGCTACCTACTATTGCCAGCAATCCCTG |
| | GCCGACCCCTTCACATTCGGCGGAGGCACAAAGGTTGAGATCAAGGCTGCTG |
| | CATTGGATAATGAAAAATCGAACGGCACAATCATTCATGTGAAGGGCAAAC |
| | ACCTGTGTCCCAGCCCCTTGTTCCCAGGACCTAGCAAGCCTTTTTGGGTTCTC |
| | GTGGTGGTGGGCGGCGTTCTGGCTTGCTACTCTCTACTTGTAACTGTCGCATT |
| | TATTATATTCTGGGTTAGATTCAGCGTTGTGAAGAGAGGCCGGAAGAAGCTG |
| | CTGTACATCTTCAAGCAGCCCTTCATGAGACCTGTGCAGACCACACAGGAGG |
| | AAGACGGCTGCAGCTGTAGATTCCCCGAGGAAGAGGAGGGCGGCTGTGAGC |
| | TGAGAGTTAAGTTCAGCAGGAGCGCCGACGCCCCTGCCTACCAGCAAGGAC |
| | AGAATCAACTGTACAACGAGCTGAACCTGGGCAGACGGGAGGAATACGATG |
| | TGCTGGACAAGAGGAGAGGCAGAGACCCCGAGATGGGCGGCAAACCTAGA |
| | AGAAAGAACCCCCAGGAGGGCCTGTATAACGAGCTCCAGAAGGACAAGATG |
| | GCCGAGGCCTACAGCGAGATCGGCATGAAGGGCGAAAGAAGAAGAGGCAA |
| | GGGCCACGACGGCCTCTACCAGGGCTTAAGCACAGCTACAAAGGACACCTA |
| | CGACGCCCTGCACATGCAGGCCCTGCCCCCTAGATGATTAATTAAatcgat |
| 53 | ggtaccCCCGGgCCCATGGCTCTTCCTGTGACAGCTCTTCTGCTGCCCCTGGCCC |
| | TGCTTCTGCATGCTGCTAGACCTCAGATCACATTAAAAGAGAGCGGACCTAC |
| | ACTGGTGAAGCCCACCCAAACGCTTACCCTCACCTGCACCTTTAGCGGGTTC |
| | AGCCTGGACACAGAGGGCGTTGGCGTTGGATGGATCAGGCAGCCTCCTGGC |
| | AAAGCCCTCGAATGGCTTGCCCTCATCTACTTCAACGACCAGAAGAGATACA |
| | GCCCCTCCTTAAAATCTCGGCTCACAATCACCAAAGACACAAGCAAAAATCA |
| | GGTTGTGCTCACCATGACCAACATGGACCCTGTGGACACCGCTGTGTACTAC |
| | TGTGCCAGAGACACCGGCTACAGCAGATGGTACTACGGGATGGACGTTTGG |
| | GGCCAAGGCACCACTGTGACCGTTTCCAGCGGCTCTACAAGCGGCAGCGGG |
| | AAACCTGGTTCTGGAGAGGGCAGCACAAAGGGCGACATCCAGATGACGCAA |
| | TCCCCCAGCTCTGTGAGCGCCTCTGTGGGAGACAGAGTTACAATCACATGCC |
| | GGGCCTCCCAGGGCATCAGCTCTTGGCTGGCATGGTATCAACAGAAGCCTGG |
| | CAAGGCTCCCAAGCTGCTCATCTATGCCGCCTCCTCCTTACAATCTGGAGTTC |
| | CCTCCAGGTTCAGCGGGAGCGGCTCAGGAACAGACTTCACCCTTACCATCTC |
| | TAGCCTGCAACCCGAGGACTTCGCTACTTATTACTGTCAGCAGGCCTACGCC |
| | TACCCCATCACATTCGGCGGAGGAACAAAGGTTGAGATCAAGGCTGCTGCAT |
| | TGGATAATGAAAAATCGAACGGCACAATCATTCATGTGAAGGGCAAACACC |
| | TGTGTCCCAGCCCCTTGTTCCCAGGACCTAGCAAGCCTTTTTGGGTTCTCGTG |
| | GTGGTGGGCGGCGTTCTGGCTTGCTACTCTCTACTTGTAACTGTCGCATTTAT |
| | TATATTCTGGGTTAGATTCAGCGTTGTGAAGAGAGGCCGGAAGAAGCTGCTG |
| | TACATCTTCAAGCAGCCCTTCATGAGACCTGTGCAGACCACACAGGAGGAAG |
| | ACGGCTGCAGCTGTAGATTCCCCGAGGAAGAGGAGGGCGGCTGTGAGCTGA |
| | GAGTTAAGTTCAGCAGGAGCGCCGACGCCCCTGCCTACCAGCAAGGACAGA |
| | ATCAACTGTACAACGAGCTGAACCTGGGCAGACGGGAGGAATACGATGTGC |
| | TGGACAAGAGGAGAGGCAGAGACCCCGAGATGGGCGGCAAACCTAGAAGA |
| | AAGAACCCCCAGGAGGGCCTGTATAACGAGCTCCAGAAGGACAAGATGGCC |
| | GAGGCCTACAGCGAGATCGGCATGAAGGGCGAAAGAAGAAGAGGCAAGGG |
| | CCACGACGGCCTCTACCAGGGCTTAAGCACAGCTACAAAGGACACCTACGA |
| | CGCCCTGCACATGCAGGCCCTGCCCCCTAGATGATTAATTAAatcgat |
| 54 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCCAGAAATACTACTGGAGCTGGATCCGGCAGCCTCCCGGCAAAGGCT |
| | TAGAATGGATCGGAGAGATAGACACCAGCGGCTTCACCAACTACAACCCCA |
| | GCCTGAAATCTAGGGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGAGTTGGCAGATACAGCTACGGCTACTACATCACCGCCTTCGACATTTGGG |
| | GCCAAGGCACCACTGTGACCGTTTCCAGCGGAAGCACTAGCGGCAGCGGGA |
| | AACCTGGTTCTGGAGAGGGCTCAACCAAGGGCGACATCGTGATGACACAGA |
| | GCCCCGACTCTCTGGCTGTGTCCCTGGGAGAGAGAGCCACCATCAACTGCAA |
| | GAGCAGCCAGAGCGTTCTGTACAGCAGCAACAACAAGAACTACCTGGCATG |
| | GTATCAACAGAAGCCTGGCCAGCCCCCTAAGCTGCTCATCTACTGGGCTTCC |
| | ACCAGAGAATCAGGCGTTCCAGACAGGTTCTCCGGCTCGGGTTCAGGCACAG |
| | ACTTCACCCTTACCATCTCTTCCCTGCAGGCCGAAGATGTGGCCGTTTACTAC |
| | TGTCAGCAGCACTACAGCTTCCCTTTCACATTCGGCGGAGGCACCAAGGTTG |
| | AGATCAAGGCTGCTGCATTGGATAATGAAAAATCGAACGGCACAATCATTC |
| | ATGTGAAGGGCAAACACCTGTGTCCCAGCCCCTTGTTCCCAGGACCTAGCAA |
| | GCCTTTTTGGGTTCTCGTGGTGGTGGGCGGCGTTCTGGCTTGCTACTCTCTAC |
| | TTGTAACTGTCGCATTTATTATATTCTGGGTTAGATTCAGCGTTGTGAAGAGA |
| | GGCCGGAAGAAGCTGCTGTACATCTTCAAGCAGCCCTTCATGAGACCTGTGC |
| | AGACCACACAGGAGGAAGACGGCTGCAGCTGTAGATTCCCCGAGGAAGAGG |
| | AGGGCGGCTGTGAGCTGAGAGTTAAGTTCAGCAGGAGCGCCGACGCCCCTG |
| | CCTACCAGCAAGGACAGAATCAACTGTACAACGAGCTGAACCTGGGCAGAC |
| | GGGAGGAATACGATGTGCTGGACAAGAGGAGAGGCAGAGACCCCGAGATG |
| | GGCGGCAAACCTAGAAGAAAGAACCCCCAGGAGGGCCTGTATAACGAGCTC |
| | CAGAAGGACAAGATGGCCGAGGCCTACAGCGAGATCGGCATGAAGGGCGA |
| | AAGAAGAAGAGGCAAGGGCCACGACGGCCTCTACCAGGGCTTAAGCACAGC |
| | TACAAAGGACACCTACGACGCCCTGCACATGCAGGCCCTGCCCCCTAGATGA |
| | TTAATTAAatcgat |
| 55 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCGAGAAATACTACTGGAGCTGGATCCGGCAGCCTCCCGGCAAAGGCT |
| | TAGAATGGATCGGCGAGATTTATCACAGCGGGCTCACCAACTACAACCCCAG |
| | CCTGAAATCTCGAGTTACAATCTCTGTGGACACAAGCAAGAATCAGTTCTCC |
| | CTGAAGCTGAGCAGCGTTACTGCCGCCGACACAGCTGTGTACTATTGCGCCA |
| | GAGTTAGATACGACAGCAGCGACAGCTATTACTACAGCTATGACTACGGCAT |
| | GGATGTGTGGGGGCAGGGCACCACCGTTACTGTCTCCTCTGGATCTACCAGC |
| | GGCAGCGGCAAGCCTGGATCTGGCGAAGGAAGCACAAAGGGCGACATTGTG |
| | CTCACCCAGAGCCCCGACAGCCTGGCTGTGTCTTTAGGCGAAAGGGCTACCA |
| | TCAACTGCAAGAGCAGCCAGAGCGTTCTGTACAGCAGCAACAACAAGAACT |
| | ACCTTGCTTGGTATCAACAGAAGCCTGGCCAGCCCCCTAAGCTGCTCATCTA |
| | CTGGGCCTCTAGCAGAGAGAGCGGGGTTCCCGATCGGTTTAGCGGCTCCGGC |
| | TCAGGAACCGATTTCACCCTCACTATCTCCAGCCTCCAGGCCGAGGATGTGG |
| | CTGTCTACTATTGTCAGCAGAGCTATAGCTTCCCCTGGACATTCGGCGGAGG |
| | CACCAAGGTTGAGATCAAGGCTGCTGCATTGGATAATGAAAAATCGAACGG |
| | CACAATCATTCATGTGAAGGGCAAACACCTGTGTCCCAGCCCCTTGTTCCCA |
| | GGACCTAGCAAGCCTTTTTGGGTTCTCGTGGTGGTGGGCGGCGTTCTGGCTT |
| | GCTACTCTCTACTTGTAACTGTCGCATTTATTATATTCTGGGTTAGATTCAGC |
| | GTTGTGAAGAGAGGCCGGAAGAAGCTGCTGTACATCTTCAAGCAGCCCTTCA |
| | TGAGACCTGTGCAGACCACACAGGAGGAAGACGGCTGCAGCTGTAGATTCC |
| | CCGAGGAAGAGGAGGGCGGCTGTGAGCTGAGAGTTAAGTTCAGCAGGAGCG |
| | CCGACGCCCCTGCCTACCAGCAAGGACAGAATCAACTGTACAACGAGCTGA |
| | ACCTGGGCAGACGGGAGGAATACGATGTGCTGGACAAGAGGAGAGGCAGA |
| | GACCCCGAGATGGGCGGCAAACCTAGAAGAAAGAACCCCCAGGAGGGCCTG |
| | TATAACGAGCTCCAGAAGGACAAGATGGCCGAGGCCTACAGCGAGATCGGC |
| | ATGAAGGGCGAAAGAAGAAGAGGCAAGGGCCACGACGGCCTCTACCAGGG |
| | CTTAAGCACAGCTACAAAGGACACCTACGACGCCCTGCACATGCAGGCCCTG |
| | CCCCCTAGATGATTAATTAAatcgat |
| 56 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCAGCCGCTATGTGTGGAGCTGGATCCGGCAGCCTCCTGGCAAAGGCC |
| | TTGAATGGATCGGAGAGATAGACAGCAGCGGCAAGACCAACTACAACCCCA |
| | GCCTGAAATCACGCGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGAGTTAGATACGACAGCTCCGACAGCTATTACTACAGCTATGACTACGGCA |
| | TGGATGTGTGGGGGCAGGGCACCACCGTTACAGTTAGCTCTGGAAGCACCA |
| | GCGGCTCCGGCAAGCCTGGATCTGGTGAAGGAAGCACAAAGGGCGACATTG |
| | TGCTCACCCAGAGCCCCGACAGCCTGGCTGTGTCTTTAGGCGAAAGGGCTAC |
| | CATCAACTGCAAGAGCAGCCAGAGCGTTCTGTACAGCAGCAACAACAAGAA |
| | CTACCTTGCATGGTATCAACAGAAGCCTGGCCAGCCTCCCAAGCTGCTCATC |
| | TACTGGGCCTCTAGCAGAGAGAGCGGGGTTCCCGATCGCTTTAGCGGCAGCG |
| | GTTCTGGCACCGATTTCACTCTTACAATCAGCAGCTTACAGGCCGAGGATGT |
| | GGCTGTCTACTATTGTCAGCAGAGCTATAGCTTCCCCTGGACATTCGGCGGA |
| | GGCACCAAGGTTGAGATCAAGGCTGCTGCATTGGATAATGAAAAATCGAAC |
| | GGCACAATCATTCATGTGAAGGGCAAACACCTGTGTCCCAGCCCCTTGTTCC |
| | CAGGACCTAGCAAGCCTTTTTGGGTTCTCGTGGTGGTGGGCGGCGTTCTGGC |
| | TTGCTACTCTCTACTTGTAACTGTCGCATTTATTATATTCTGGGTTAGATTCA |
| | GCGTTGTGAAGAGAGGCCGGAAGAAGCTGCTGTACATCTTCAAGCAGCCCTT |
| | CATGAGACCTGTGCAGACCACACAGGAGGAAGACGGCTGCAGCTGTAGATT |
| | CCCCGAGGAAGAGGAGGGCGGCTGTGAGCTGAGAGTTAAGTTCAGCAGGAG |
| | CGCCGACGCCCCTGCCTACCAGCAAGGACAGAATCAACTGTACAACGAGCT |
| | GAACCTGGGCAGACGGGAGGAATACGATGTGCTGGACAAGAGGAGAGGCA |
| | GAGACCCCGAGATGGGCGGCAAACCTAGAAGAAAGAACCCCCAGGAGGGC |
| | CTGTATAACGAGCTCCAGAAGGACAAGATGGCCGAGGCCTACAGCGAGATC |
| | GGCATGAAGGGCGAAAGAAGAAGAGGCAAGGGCCACGACGGCCTCTACCA |
| | GGGCTTAAGCACAGCTACAAAGGACACCTACGACGCCCTGCACATGCAGGC |
| | CCTGCCCCCTAGATGATTAATTAAatcgat |
| 57 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCAGCGGCTACGCTTGGAGCTGGATTAGACAGCCTCCTGGCAAAGGAC |
| | TAGAATGGATCGGAGAGATCGACCACAGAGGCTTCACCAACTACAACCCCA |
| | GCCTGAAATCCAGAGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGGGTTAGATACGACAGCAGCGACAGCTATTACTACAGCTATGACTACGGC |
| | ATGGATGTGTGGGGGCAGGGCACCACCGTTACGGTTAGCTCTGGATCTACCA |
| | GCGGCAGCGGCAAGCCTGGCTCAGGAGAAGGAAGCACAAAGGGCGACATTG |
| | TGCTCACCCAGAGCCCCGACAGCCTGGCCGTTTCTTTAGGCGAAAGGGCTAC |
| | CATCAACTGCAAGAGCAGCCAGAGCGTTCTGTACAGCAGCAACAACAAGAA |
| | CTACCTTGCATGGTATCAACAGAAGCCAGGCCAGCCTCCCAAGCTGCTCATC |
| | TACTGGGCCTCTAGCAGAGAGAGCGGGGTTCCCGATAGATTTTCGGGATCAG |
| | GCTCCGGCACCGATTTCACTCTTACGATCAGCAGCTTACAGGCCGAGGATGT |
| | GGCTGTCTACTATTGTCAGCAGAGCTATAGCTTCCCCTGGACATTCGGCGGA |
| | GGCACCAAGGTTGAGATCAAGGCTGCTGCATTGGATAATGAAAAATCGAAC |
| | GGCACAATCATTCATGTGAAGGGCAAACACCTGTGTCCCAGCCCCTTGTTCC |
| | CAGGACCTAGCAAGCCTTTTTGGGTTCTCGTGGTGGTGGGCGGCGTTCTGGC |
| | TTGCTACTCTCTACTTGTAACTGTCGCATTTATTATATTCTGGGTTAGATTCA |
| | GCGTTGTGAAGAGAGGCCGGAAGAAGCTGCTGTACATCTTCAAGCAGCCCTT |
| | CATGAGACCTGTGCAGACCACACAGGAGGAAGACGGCTGCAGCTGTAGATT |
| | CCCCGAGGAAGAGGAGGGCGGCTGTGAGCTGAGAGTTAAGTTCAGCAGGAG |
| | CGCCGACGCCCCTGCCTACCAGCAAGGACAGAATCAACTGTACAACGAGCT |
| | GAACCTGGGCAGACGGGAGGAATACGATGTGCTGGACAAGAGGAGAGGCA |
| | GAGACCCCGAGATGGGCGGCAAACCTAGAAGAAAGAACCCCCAGGAGGGC |
| | CTGTATAACGAGCTCCAGAAGGACAAGATGGCCGAGGCCTACAGCGAGATC |
| | GGCATGAAGGGCGAAAGAAGAAGAGGCAAGGGCCACGACGGCCTCTACCA |
| | GGGCTTAAGCACAGCTACAAAGGACACCTACGACGCCCTGCACATGCAGGC |
| | CCTGCCCCCTAGATGATTAATTAAatcgat |
| 58 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCAGCGGCTATTACTGGAGCTGGATCCGGCAGCCTCCTGGAAAAGGAT |
| | TAGAATGGATCGGCGAGATAGACCACAGCGGGAGCACAAACTACAACCCCA |
| | GCCTGAAATCGCGGGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGAGGCGGAGGCTCCTGGTACAGCAACTGGTTCGATCCTTGGGGCCAAGGC |
| | ACCATGGTGACCGTTTCCAGCGGCTCTACAAGCGGCAGCGGGAAACCTGGTT |
| | CTGGAGAGGGCAGCACAAAGGGCGACATCCAGATGACACAGAGCCCCAGCA |
| | CCCTTAGCGCCTCTGTGGGAGATAGGGTTACCATTACCTGCAGGGCTTCCCA |
| | GAGCATCAGCAGCTGGCTGGCATGGTATCAACAGAAGCCTGGCAAGGCTCC |
| | CAAGCTGCTCATCTATGACGCCTCCAGCCTGGAAAGCGGGGTTCCCTCCAGA |
| | TTTAGCGGCTCAGGCTCCGGAACAGAGTTCACCCTTACCATCTCTAGCCTGC |
| | AACCCGACGACTTCGCTACTTATTACTGTCAACAAGACAGAAGCTTGCCCCC |
| | CACATTCGGCGGAGGGACCAAGGTTGAGATCAAGGCTGCTGCATTGGATAA |
| | TGAAAAATCGAACGGCACAATCATTCATGTGAAGGGCAAACACCTGTGTCCC |
| | AGCCCCTTGTTCCCAGGACCTAGCAAGCCTTTTTGGGTTCTCGTGGTGGTGGG |
| | CGGCGTTCTGGCTTGCTACTCTCTACTTGTAACTGTCGCATTTATTATATTCTG |
| | GGTTAGATTCAGCGTTGTGAAGAGAGGCCGGAAGAAGCTGCTGTACATCTTC |
| | AAGCAGCCCTTCATGAGACCTGTGCAGACCACACAGGAGGAAGACGGCTGC |
| | AGCTGTAGATTCCCCGAGGAAGAGGAGGGCGGCTGTGAGCTGAGAGTTAAG |
| | TTCAGCAGGAGCGCCGACGCCCCTGCCTACCAGCAAGGACAGAATCAACTG |
| | TACAACGAGCTGAACCTGGGCAGACGGGAGGAATACGATGTGCTGGACAAG |
| | AGGAGAGGCAGAGACCCCGAGATGGGCGGCAAACCTAGAAGAAAGAACCC |
| | CCAGGAGGGCCTGTATAACGAGCTCCAGAAGGACAAGATGGCCGAGGCCTA |
| | CAGCGAGATCGGCATGAAGGGCGAAAGAAGAAGAGGCAAGGGCCACGACG |
| | GCCTCTACCAGGGCTTAAGCACAGCTACAAAGGACACCTACGACGCCCTGCA |
| | CATGCAGGCCCTGCCCCCTAGATGATTAATTAAatcgat |
| 59 | ggtaccCCCGGgCCCATGGCTCTTCCTGTGACAGCTCTTCTGCTGCCCCTGGCCC |
| | TGCTTCTGCATGCTGCTAGACCTCAGGTTCAGCTTGTGCAGAGCGGAGCTGA |
| | AGTTAAGAAGCCTGGCGCCTCTGTGAAGGTTAGCTGCAAGGCCAGCGGCTAC |
| | ACATTCAAGGAATATGGCATCTCCTGGGTTAGGCAGGCTCCCGGCCAAGGCT |
| | TAGAATGGATGGGCTGGATCTCCGCCTACTCCGGCCACACCTACTACGCCCA |
| | GAAGCTTCAGGGCAGGGTTACCATGACCACCGACACCAGCACCTCTACCGCC |
| | TATATGGAGCTGAGGAGCCTGAGATCGGACGACACAGCTGTGTATTACTGCG |
| | CCAGAGGCCCCCACTACGACGACTGGTCTGGATTTATCATCTGGTTCGACCC |
| | CTGGGGGCAGGGCACCCTGGTCACAGTTTCTTCTGGCTCCACCAGCGGAAGC |
| | GGCAAGCCAGGCTCAGGCGAAGGATCTACAAAAGGCGACATCCAAATGACA |
| | CAGAGCCCCAGCAGCTTGAGCGCCTCCGTTGGCGACAGAGTTACAATCACCT |
| | GCAGGGCCTCTCAGAGCATCAGCAGCTATTTGAATTGGTATCAACAGAAGCC |
| | AGGAAAGGCCCCTAAGCTGCTCATCTACGCTGCCAGCTCGCTCCAATCTGGC |
| | GTTCCTAGCAGATTTAGCGGCTCCGGCAGCGGCACAGACTTTACTCTTACCA |
| | TTAGCTCCCTGCAGCCCGAGGACTTCGCTACCTACTATTGCCAGCAAAGCTA |
| | CAGATTCCCTCCCACCTTTGGCCAGGGCACAAAGGTTGAGATCAAGGCTGCT |
| | GCATTGGATAATGAAAAATCGAACGGCACAATCATTCATGTGAAGGGCAAA |
| | CACCTGTGTCCCAGCCCCTTGTTCCCAGGACCTAGCAAGCCTTTTTGGGTTCT |
| | CGTGGTGGTGGGCGGCGTTCTGGCTTGCTACTCTCTACTTGTAACTGTCGCAT |
| | TTATTATATTCTGGGTTAGATTCAGCGTTGTGAAGAGAGGCCGGAAGAAGCT |
| | GCTGTACATCTTCAAGCAGCCCTTCATGAGACCTGTGCAGACCACACAGGAG |
| | GAAGACGGCTGCAGCTGTAGATTCCCCGAGGAAGAGGAGGGCGGCTGTGAG |
| | CTGAGAGTTAAGTTCAGCAGGAGCGCCGACGCCCCTGCCTACCAGCAAGGA |
| | CAGAATCAACTGTACAACGAGCTGAACCTGGGCAGACGGGAGGAATACGAT |
| | GTGCTGGACAAGAGGAGAGGCAGAGACCCCGAGATGGGCGGCAAACCTAG |
| | AAGAAAGAACCCCCAGGAGGGCCTGTATAACGAGCTCCAGAAGGACAAGAT |
| | GGCCGAGGCCTACAGCGAGATCGGCATGAAGGGCGAAAGAAGAAGAGGCA |
| | AGGGCCACGACGGCCTCTACCAGGGCTTAAGCACAGCTACAAAGGACACCT |
| | ACGACGCCCTGCACATGCAGGCCCTGCCCCCTAGATGATTAATTAAatcgat |
| 60 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTGCAGCAATGGGGAGCTGG |
| | CCTGTTAAAGCCCAGCGAAACCCTGTCCCTCACCTGCGCTGTGTATGGCGGA |
| | AGCTTCAGCGGCATCCACTGGAACTGGATCCGGCAGCCTCCTGGCAAAGGCC |
| | TTGAATGGATCGGCGATATCGACACCAGCGGCTCCACCAACTACAACCCCAG |
| | CCTGAAATCGAGGGTTACAATCTCTGTGGACACAAGCAAGAATCAGTTCTCC |
| | CTGAAGCTGAGCAGCGTTACTGCCGCCGACACAGCTGTGTACTATTGCGCCA |
| | GACTGGGCCAGGAAAGCGCTACCTACCTTGGCATGGATGTGTGGGGGCAGG |
| | GCACCACCGTTACTGTTAGCTCTGGCTCAACAAGCGGCAGCGGCAAGCCTGG |
| | CTCAGGAGAAGGAAGCACAAAGGGCGACATTGTAATGACTCAGAGCCCCGA |
| | CAGCCTGGCCGTTAGCTTAGGCGAAAGGGCTACAATCAATTGCAAGAGCAG |
| | CCAGAGCGTTCTGTACAGCAGCAACAACAAGAACTACCTCGCATGGTATCAA |
| | CAGAAGCCAGGCCAGCCTCCCAAGCTGCTCATCTACTGGGCTTCCACCAGAG |
| | AGAGCGGGGTTCCCGATAGATTCTCCGGCTCCGGTTCTGGAACAGATTTCAC |
| | GCTCACAATCAGCAGCTTACAGGCCGAGGATGTGGCTGTCTACTATTGTCAG |
| | CAGTTGTACACCTACCCCTTCACATTCGGCGGAGGCACCAAGGTTGAGATCA |
| | AGGCTGCTGCATTGGATAATGAAAAATCGAACGGCACAATCATTCATGTGAA |
| | GGGCAAACACCTGTGTCCCAGCCCCTTGTTCCCAGGACCTAGCAAGCCTTTT |
| | TGGGTTCTCGTGGTGGTGGGCGGCGTTCTGGCTTGCTACTCTCTACTTGTAAC |
| | TGTCGCATTTATTATATTCTGGGTTAGATTCAGCGTTGTGAAGAGAGGCCGG |
| | AAGAAGCTGCTGTACATCTTCAAGCAGCCCTTCATGAGACCTGTGCAGACCA |
| | CACAGGAGGAAGACGGCTGCAGCTGTAGATTCCCCGAGGAAGAGGAGGGCG |
| | GCTGTGAGCTGAGAGTTAAGTTCAGCAGGAGCGCCGACGCCCCTGCCTACCA |
| | GCAAGGACAGAATCAACTGTACAACGAGCTGAACCTGGGCAGACGGGAGGA |
| | ATACGATGTGCTGGACAAGAGGAGAGGCAGAGACCCCGAGATGGGCGGCAA |
| | ACCTAGAAGAAAGAACCCCCAGGAGGGCCTGTATAACGAGCTCCAGAAGGA |
| | CAAGATGGCCGAGGCCTACAGCGAGATCGGCATGAAGGGCGAAAGAAGAA |
| | GAGGCAAGGGCCACGACGGCCTCTACCAGGGCTTAAGCACAGCTACAAAGG |
| | ACACCTACGACGCCCTGCACATGCAGGCCCTGCCCCCTAGATGATTAATTAA |
| | atcgat |
| 61 | ggtaccCCCGGgCCCATGGCTCTTCCTGTGACAGCTCTTCTGCTGCCCCTGGCCC |
| | TGCTTCTGCATGCTGCTAGACCTCAGCTTCAGCTCCAAGAGAGCGGACCTGG |
| | CTTAGTGAAGCCCAGCGAAACCCTGTCCCTCACCTGCACCGTTTCTGGCGGA |
| | AGCATCAGCAGCTCCAGCTATTACTGGGGATGGATCAGGCAGCCCCCTGGCA |
| | AGGGTTTAGAATGGATCGGCTCGATATATTACTCCGGCAGCACCTACTATAA |
| | CCCCAGCTTGAAGAGCCGGGTTACCATTTCTGTGGACACATCAAAGAACCAG |
| | TTCAGCCTGAAGCTGAGCTCTGTGACTGCCGCCGACACAGCTGTGTACTACT |
| | GTGCCAGAGAGACAGACTACTCCAGCGGCATGGGCTACGGCATGGATGTGT |
| | GGGGACAAGGAACCACCGTTACTGTGAGCAGCGGTTCCACCAGCGGCTCAG |
| | GCAAGCCTGGCTCAGGAGAAGGAAGCACCAAGGGGGATATACAGATGACAC |
| | AGAGCCCCTCCAGCCTGTCCGCCAGCGTTGGCGATCGTGTAACGATCACCTG |
| | CCGGGCCTCTCAGAGCATCAACTCCTACCTCAATTGGTATCAACAGAAGCCA |
| | GGCAAGGCCCCCAAATTACTCATCTACGCCGCCAGCAGCTTACAGAGCGGG |
| | GTTCCCTCTAGATTCTCCGGCTCCGGTTCTGGAACAGATTTCACCCTCACTAT |
| | CTCCAGCTTGCAGCCCGAGGATTTCGCCACTTATTACTGTCAGCAGAGCCTG |
| | GCCGACCCCTTCACATTCGGCGGAGGCACAAAGGTTGAGATCAAGGCAGCT |
| | GCTTTCGTGCCTGTGTTCCTGCCTGCTAAGCCCACCACCACTCCTGCTCCAAG |
| | ACCTCCTACCCCCGCTCCTACAATCGCCAGCCAACCTCTGAGCCTGAGACCG |
| | GAGGCATGCAGACCTGCGGCAGGGGGAGCAGTTCACACAAGAGGCTTGGAC |
| | TTCGCTTGCGACATCTACATCTGGGCCCCTCTGGCCGGCACATGCGGAGTTCT |
| | TCTTCTTAGCCTGGTGATCACCCTGTACTGCAACCACAGAAACAGATTCAGC |
| | GTTGTGAAGAGAGGCCGGAAGAAGCTGCTGTACATCTTCAAGCAGCCCTTCA |
| | TGAGACCTGTGCAGACCACACAGGAGGAAGACGGCTGCAGCTGTAGATTCC |
| | CCGAGGAAGAGGAGGGCGGCTGTGAGCTGAGAGTTAAGTTCAGCAGGAGCG |
| | CCGACGCCCCTGCCTACCAGCAAGGACAGAATCAACTGTACAACGAGCTGA |
| | ACCTGGGCAGACGGGAGGAATACGATGTGCTGGACAAGAGGAGAGGCAGA |
| | GACCCCGAGATGGGCGGCAAACCTAGAAGAAAGAACCCCCAGGAGGGCCTG |
| | TATAACGAGCTCCAGAAGGACAAGATGGCCGAGGCCTACAGCGAGATCGGC |
| | ATGAAGGGCGAAAGAAGAAGAGGCAAGGGCCACGACGGCCTCTACCAGGG |
| | CTTAAGCACAGCTACAAAGGACACCTACGACGCCCTGCACATGCAGGCCCTG |
| | CCCCCTAGATGATTAATTAAatcgat |
| 62 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAAGAGAGCGGACCTGG |
| | CTTAGTGAAGCCCAGCGAAACCCTGTCCCTCACCTGCACCGTTTCTGGCGGA |
| | AGCATCAGCTCTCCCGACCATTACTGGGGATGGATCAGGCAGCCCCCTGGCA |
| | AGGGTTTGGAATGGATCGGCAGCATCTACGCCAGCGGCAGCACATTCTACAA |
| | CCCCTCGCTCAAAAGCAGGGTTACTATTTCTGTGGACACAAGCAAAAATCAG |
| | TTCAGCCTGAAGCTGAGCTCTGTGACTGCCGCCGACACAGCTGTGTACTACT |
| | GTGCCAGAGAGACAGACTACTCCAGCGGGATGGGCTACGGCATGGATGTGT |
| | GGGGACAAGGAACCACCGTTACTGTGAGCAGCGGCTCCACAAGCGGCTCAG |
| | GCAAGCCTGGCTCAGGAGAAGGAAGCACCAAGGGGGACATTCAAATGACCC |
| | AAAGCCCCTCCAGCCTGTCCGCCAGCGTTGGCGATAGGGTTACCATTACCTG |
| | CAGGGCCAGCCAAAGCATCAACTCCTACCTAAATTGGTATCAACAGAAGCC |
| | AGGCAAGGCCCCCAAACTACTCATTTACGCCGCCAGCAGCTTACAGAGCGG |
| | GGTTCCCTCTAGATTCTCCGGCAGCGGTTCTGGAACAGATTTCACTCTCACAA |
| | TATCTTCGCTGCAGCCCGAGGATTTCGCTACCTACTATTGCCAGCAATCCCTG |
| | GCCGACCCCTTCACATTCGGCGGAGGCACAAAGGTTGAGATCAAGGCAGCT |
| | GCTTTCGTGCCTGTGTTCCTGCCTGCTAAGCCCACCACCACTCCTGCTCCAAG |
| | ACCTCCTACCCCCGCTCCTACAATCGCCAGCCAACCTCTGAGCCTGAGACCG |
| | GAGGCATGCAGACCTGCGGCAGGGGGAGCAGTTCACACAAGAGGCTTGGAC |
| | TTCGCTTGCGACATCTACATCTGGGCCCCTCTGGCCGGCACATGCGGAGTTCT |
| | TCTTCTTAGCCTGGTGATCACCCTGTACTGCAACCACAGAAACAGATTCAGC |
| | GTTGTGAAGAGAGGCCGGAAGAAGCTGCTGTACATCTTCAAGCAGCCCTTCA |
| | TGAGACCTGTGCAGACCACACAGGAGGAAGACGGCTGCAGCTGTAGATTCC |
| | CCGAGGAAGAGGAGGGCGGCTGTGAGCTGAGAGTTAAGTTCAGCAGGAGCG |
| | CCGACGCCCCTGCCTACCAGCAAGGACAGAATCAACTGTACAACGAGCTGA |
| | ACCTGGGCAGACGGGAGGAATACGATGTGCTGGACAAGAGGAGAGGCAGA |
| | GACCCCGAGATGGGCGGCAAACCTAGAAGAAAGAACCCCCAGGAGGGCCTG |
| | TATAACGAGCTCCAGAAGGACAAGATGGCCGAGGCCTACAGCGAGATCGGC |
| | ATGAAGGGCGAAAGAAGAAGAGGCAAGGGCCACGACGGCCTCTACCAGGG |
| | CTTAAGCACAGCTACAAAGGACACCTACGACGCCCTGCACATGCAGGCCCTG |
| | CCCCCTAGATGATTAATTAAatcgat |
| 63 | ggtaccCCCGGgCCCATGGCTCTTCCTGTGACAGCTCTTCTGCTGCCCCTGGCCC |
| | TGCTTCTGCATGCTGCTAGACCTCAGATCACATTAAAAGAGAGCGGACCTAC |
| | ACTGGTGAAGCCCACCCAAACGCTTACCCTCACCTGCACCTTTAGCGGGTTC |
| | AGCCTGGACACAGAGGGCGTTGGCGTTGGATGGATCAGGCAGCCTCCTGGC |
| | AAAGCCCTCGAATGGCTTGCCCTCATCTACTTCAACGACCAGAAGAGATACA |
| | GCCCCTCCTTAAAATCTCGGCTCACAATCACCAAAGACACAAGCAAAAATCA |
| | GGTTGTGCTCACCATGACCAACATGGACCCTGTGGACACCGCTGTGTACTAC |
| | TGTGCCAGAGACACCGGCTACAGCAGATGGTACTACGGGATGGACGTTTGG |
| | GGCCAAGGCACCACTGTGACCGTTTCCAGCGGCTCTACAAGCGGCAGCGGG |
| | AAACCTGGTTCTGGAGAGGGCAGCACAAAGGGCGACATCCAGATGACGCAA |
| | TCCCCCAGCTCTGTGAGCGCCTCTGTGGGAGACAGAGTTACAATCACATGCC |
| | GGGCCTCCCAGGGCATCAGCTCTTGGCTGGCATGGTATCAACAGAAGCCTGG |
| | CAAGGCTCCCAAGCTGCTCATCTATGCCGCCTCCTCCTTACAATCTGGAGTTC |
| | CCTCCAGGTTCAGCGGGAGCGGCTCAGGAACAGACTTCACCCTTACCATCTC |
| | TAGCCTGCAACCCGAGGACTTCGCTACTTATTACTGTCAGCAGGCCTACGCC |
| | TACCCCATCACATTCGGCGGAGGAACAAAGGTTGAGATCAAGGCAGCTGCTT |
| | TCGTGCCTGTGTTCCTGCCTGCTAAGCCCACCACCACTCCTGCTCCAAGACCT |
| | CCTACCCCCGCTCCTACAATCGCCAGCCAACCTCTGAGCCTGAGACCGGAGG |
| | CATGCAGACCTGCGGCAGGGGGAGCAGTTCACACAAGAGGCTTGGACTTCG |
| | CTTGCGACATCTACATCTGGGCCCCTCTGGCCGGCACATGCGGAGTTCTTCTT |
| | CTTAGCCTGGTGATCACCCTGTACTGCAACCACAGAAACAGATTCAGCGTTG |
| | TGAAGAGAGGCCGGAAGAAGCTGCTGTACATCTTCAAGCAGCCCTTCATGA |
| | GACCTGTGCAGACCACACAGGAGGAAGACGGCTGCAGCTGTAGATTCCCCG |
| | AGGAAGAGGAGGGCGGCTGTGAGCTGAGAGTTAAGTTCAGCAGGAGCGCCG |
| | ACGCCCCTGCCTACCAGCAAGGACAGAATCAACTGTACAACGAGCTGAACC |
| | TGGGCAGACGGGAGGAATACGATGTGCTGGACAAGAGGAGAGGCAGAGAC |
| | CCCGAGATGGGCGGCAAACCTAGAAGAAAGAACCCCCAGGAGGGCCTGTAT |
| | AACGAGCTCCAGAAGGACAAGATGGCCGAGGCCTACAGCGAGATCGGCATG |
| | AAGGGCGAAAGAAGAAGAGGCAAGGGCCACGACGGCCTCTACCAGGGCTTA |
| | AGCACAGCTACAAAGGACACCTACGACGCCCTGCACATGCAGGCCCTGCCC |
| | CCTAGATGATTAATTAAatcgat |
| 64 | ggtaccCCCGGgCCCATGGCTCTTCCTGTGACAGCTCTTCTGCTGCCCCTGGCCC |
| | TGCTTCTGCATGCTGCTAGACCTCAGGTTCAGTTACAACAATGGGGAGCTGG |
| | CCTGTTAAAGCCCAGCGAAACCCTGTCCCTCACCTGCGCTGTGTATGGCGGA |
| | AGCTTCCAGAAATACTACTGGAGCTGGATCCGGCAGCCTCCCGGCAAAGGCT |
| | TAGAATGGATCGGAGAGATAGACACCAGCGGCTTCACCAACTACAACCCCA |
| | GCCTGAAATCTAGGGTTACAATCTCTGTGGACACAAGCAAGAATCAGTTCTC |
| | CCTGAAGCTGAGCAGCGTTACTGCCGCCGACACAGCTGTGTACTATTGCGCC |
| | AGAGTTGGCAGATACAGCTACGGCTACTACATCACCGCCTTCGACATTTGGG |
| | GCCAAGGCACCACTGTGACCGTTTCCAGCGGAAGCACTAGCGGCAGCGGGA |
| | AACCTGGTTCTGGAGAGGGCTCAACCAAGGGCGACATCGTGATGACACAGA |
| | GCCCCGACTCTCTGGCTGTGTCCCTGGGAGAGAGAGCCACCATCAACTGCAA |
| | GAGCAGCCAGAGCGTTCTGTACAGCAGCAACAACAAGAACTACCTGGCATG |
| | GTATCAACAGAAGCCTGGCCAGCCCCCTAAGCTGCTCATCTACTGGGCTTCC |
| | ACCAGAGAATCAGGCGTTCCAGACAGGTTCTCCGGCTCGGGTTCAGGCACAG |
| | ACTTCACCCTTACCATCTCTTCCCTGCAGGCCGAAGATGTGGCCGTTTACTAC |
| | TGTCAGCAGCACTACAGCTTCCCTTTCACATTCGGCGGAGGCACCAAGGTTG |
| | AGATCAAGGCAGCTGCTTTCGTGCCTGTGTTCCTGCCTGCTAAGCCCACCAC |
| | CACTCCTGCTCCAAGACCTCCTACCCCCGCTCCTACAATCGCCAGCCAACCTC |
| | TGAGCCTGAGACCGGAGGCATGCAGACCTGCGGCAGGGGGAGCAGTTCACA |
| | CAAGAGGCTTGGACTTCGCTTGCGACATCTACATCTGGGCCCCTCTGGCCGG |
| | CACATGCGGAGTTCTTCTTCTTAGCCTGGTGATCACCCTGTACTGCAACCACA |
| | GAAACAGATTCAGCGTTGTGAAGAGAGGCCGGAAGAAGCTGCTGTACATCT |
| | TCAAGCAGCCCTTCATGAGACCTGTGCAGACCACACAGGAGGAAGACGGCT |
| | GCAGCTGTAGATTCCCCGAGGAAGAGGAGGGCGGCTGTGAGCTGAGAGTTA |
| | AGTTCAGCAGGAGCGCCGACGCCCCTGCCTACCAGCAAGGACAGAATCAAC |
| | TGTACAACGAGCTGAACCTGGGCAGACGGGAGGAATACGATGTGCTGGACA |
| | AGAGGAGAGGCAGAGACCCCGAGATGGGCGGCAAACCTAGAAGAAAGAAC |
| | CCCCAGGAGGGCCTGTATAACGAGCTCCAGAAGGACAAGATGGCCGAGGCC |
| | TACAGCGAGATCGGCATGAAGGGCGAAAGAAGAAGAGGCAAGGGCCACGA |
| | CGGCCTCTACCAGGGCTTAAGCACAGCTACAAAGGACACCTACGACGCCCTG |
| | CACATGCAGGCCCTGCCCCCTAGATGATTAATTAAatcgat |

A hinge may be derived from a natural source or from a synthetic source. In some embodiments, an Antigen binding system of the present disclosure may comprise a hinge that is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F 1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLA1VIF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA1-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof. In certain embodiments, a CAR does not comprise a CD28 hinge.

A transmembrane domain may be derived either from a natural or from a synthetic source. Where the source is natural, a domain may be derived from any membrane-bound or transmembrane protein. Exemplary transmembrane domains may be derived from (e.g., may comprise at least a transmembrane domain of) an alpha, beta or zeta chain of a T cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof. In some embodiments, a transmembrane domain may be synthetic (and can, e.g., comprise predominantly hydrophobic residues such as leucine and valine). In some embodiments, a triplet of phenylalanine, tryptophan and valine are comprised at each end of a synthetic transmembrane domain. In some embodiments, a transmembrane domain is directly linked or connected to a cytoplasmic domain. In some embodiments, a short oligo- or polypeptide linker (e.g., between 2 and 10 amino acids in length) may form a linkage between a transmembrane domain and an intracellular domain. In some embodiments, a linker is a glycine-serine doublet.

In some embodiments, a signaling domain and/or activation domain comprises an immunoreceptor tyrosine-based activation motif (ITAM). Examples of ITAM containing cytoplasmic signaling sequences comprise those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2:a002485 (2010); Smith-Garvin et al., Annu. Rev. Immunol. 27:591-619 (2009)).

A CAR may comprise a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), OX40, CD30, CD40, PD-1, ICOS, lymphocyte function-associated antigen-1 (LFA-1), MYD88, CD2, CD7, LIGHT, NKG2C, B7-H3, and a ligand that binds with CD83.

In certain embodiments, the CARs contemplated herein may comprise linker residues between the various domains, e.g., between VH and VL domains, added for appropriate spacing conformation of the molecule. CARs contemplated herein, may comprise one, two, three, four, or five or more linkers. In some embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In some embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. In some embodiments, a signaling domain and/or activation domain comprises an immunoreceptor tyrosine-based activation motif (ITAM). Examples of ITAM containing cytoplasmic signaling sequences comprise those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2:a002485 (2010); Smith-Garvin et al., Annu. Rev. Immunol. 27:591-619 (2009)). In certain embodiments, suitable signaling domains comprise, without limitation, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

Components of a CAR may be exchanged or “swapped” using routine techniques of biotechnology for equivalent components. In various embodiments, the present disclosure provides a binding motif according to any one of SEQ ID NOs: 31-40 or a sequence having at least 75% identity to any one of SEQ ID NOs: 31-40 (e.g., at least 80%, at least 85%, at least 90%, at least 95%, at least 96%, at least 97%, at least 98%, or at least 99% identity; e.g., 85-90%, 85-95%, 85-100%, 90-95%, 90-100%, or 95-100%) in combination with (e.g., adjacently fused to) a hinge, optionally in further combination with (e.g., adjacently fused to) a costimulatory domain. A few, non-limiting examples thereof are provided in SEQ ID NOs: 42-64 or a sequence having at least 75% identity to any one of SEQ ID NOs: 42-64 (e.g., at least 80%, at least 85%, at least 90%, at least 95%, at least 96%, at least 97%, at least 98%, or at least 99% identity; e.g., 85-90%, 85-100%, 90-95%, 90-100%, or 95-100%).

A bicistronic CAR may comprise a first CAR sequence and a second CAR sequence expressed as a single polypeptide comprising a cleavable linker between the first and second CARs. Examples of cleavable linkers include, but are not limited to, Furin-GSG-T2A (see, e.g., Chng et al. MAbs. 2015 March-April; 7(2): 403-412, which is herein incorporated by reference with respect to cleavable linkers; see also Guedan et al. Mol Ther Methods Clin Dev. 2019 Mar. 15; 12: 145-156, which is incorporated herein by reference with respect to bicistronic CAR design), 2A linkers (for example T2A), 2A-like linkers or functional equivalents thereof and combinations thereof. In some embodiments, the linkers include the picornaviral 2A-like linker, CHYSEL sequences of porcine teschovirus (P2A), virus (T2A) or combinations, variants and functional equivalents thereof.

An exemplary anti-CD20/anti-CD19 bicistronic CAR may have or comprise the nucleotide and amino acid sequences set forth in SEQ ID NOs: 28 and 26, respectively, or a sequence having at least 75% identity to SEQ ID NOs: 28 and 26 (e.g., at least 80%, at least 85%, at least 90%, at least 95%, at least 96%, at least 97%, at least 98%, or at least 99% identity; e.g., 85-90%, 85-95%, 90-95%, 90-100%, or 95-100%).

A bispecific CAR may be a single polypeptide that comprises a first binding motif and a second binding motif. An exemplary anti-CD20/anti-CD19 bispecific CAR may have or comprise the nucleotide and amino acid sequences set forth in SEQ ID NOs: 29 and 27, respectively, or a sequence having at least 75% identity to SEQ ID NOs: 29 and 27 (e.g., at least 80%, at least 85%, at least 90%, at least 95%, at least 96%, at least 97%, at least 98%, or at least 99% identity; e.g., 85-95%, 85-100%, 90-95%, 90-100%, or 95-100%).

The present disclosure provides, among other things, bispecific antibodies that bind CD20 and a second target antigen, e.g., CD19. Bispecific antibodies comprise antibodies having a first binding motif that binds a first target antigen and a second binding motif that binds a second target antigen. In some embodiments, a bispecific antibody comprises an anti-CD20 binding motif of the present disclosure and an anti-CD19 binding motif of the present disclosure. In some embodiments, a bispecific antibody comprises an anti-CD20 binding motif that comprises an anti-CD20 heavy chain variable domain of the present disclosure and an anti-CD20 light chain variable domain of the present disclosure, as well as an anti-CD19 binding motif that comprises an anti-CD19 heavy chain variable domain and an anti-CD19 light chain variable domain.

The present disclosure comprises nucleic acids encoding anti-CD20 binding motifs and/or anti-CD19 binding motifs provided herein. The present disclosure comprises nucleic acids encoding antibodies, comprising, without limitation, nucleic acids encoding binding motifs (e.g., anti-CD20 binding motifs and anti-CD19 binding motifs). The present disclosure comprises nucleic acids encoding antigen binding systems provided herein, comprising without limitation nucleic acids encoding bicistronic and bispecific chimeric antigen receptors (e.g., bicistronic and bispecific chimeric antigen receptors that bind CD20 and CD19).

The present disclosure comprises vectors that comprise nucleic acids of the present disclosure and/or that encode polypeptides of the present disclosure. In various embodiments, the present disclosure comprises a vector that comprises a nucleic acid encoding an anti-CD20 binding motif and/or an anti-CD19 binding motif provided herein. In various embodiments, the present disclosure comprises a vector that comprises a nucleic acid encoding an antibody provided herein, comprising, without limitation, a nucleic acid encoding a binding motif molecule (e.g., an anti-CD20 binding motif or an anti-CD19 binding motif). In various embodiments, the present disclosure comprises a vector that comprises a nucleic acid encoding one or more antigen binding systems provided herein, comprising without limitation nucleic acids encoding a bicistronic or bispecific chimeric antigen receptor (e.g., a bicistronic and bispecific chimeric antigen receptor that bind CD20 and CD19).

In certain embodiments, the CAR of the present disclosure binds CLL-1. Example CLL-1 CAR amino acid sequences are provided in Table 6.

TABLE 6
Exemplary CLL-1 CAR Amino Acid Sequences

| CLL-1 CAR | Sequence |
|---|---|
| CLL-1 CAR 1 | QVQLQESGPGLVKPSETLSLTCTVSGGSISSYYWSWIRQPPGKGLEWIGYI |
| | YYSGSTNYNPSLKSRVTISVDTSKNQFSLKLSSVTAADTAVYYCVSLVYC |
| | GGDCYSGFDYWGQGTLVTVSSGGGGSGGGGSGGGGSDIQLTQSPSSLSAS |
| | VGDRVSFTCQASQDINNFLNWYQQKPGKAPKLLIYDASNLETGVPSRFSG |
| | SGSGTDFTFTISSLQPEDIATYYCQQYGNLPFTFGGGTKVEIKRAAALDNE |
| | KSNGTIIHVKGKHLCPSPLFPGPSKPFWVLVVVGGVLACYSLLVTVAFIIF |
| | WVRSKRSRLLHSDYMNMTPRRPGPTRKHYQPYAPPRDFAAYRSRVKFSR |
| | SADAPAYQQGQNQLYNELNLGRREEYDVLDKRRGRDPEMGGKPRRKNP |
| | QEGLYNELQKDKMAEAYSEIGMKGERRRGKGHDGLYQGLSTATKDTYD |
| | ALHMQALPPR (SEQ ID NO. 65) |
| CLL-1 CAR 2 | QVQLQESGPGLVKPSETLSLTCTVSGGSISSYYWSWIRQPPGKGLEWIGYI |
| | YYSGSTNYNPSLKSRVTISVDTSKNQFSLKLSSVTAADTAVYYCVSLVYC |
| | GGDCYSGFDYWGQGTLVTVSSGGGGSGGGGSGGGGSDIQLTQSPSSLSAS |
| | VGDRVSFTCQASQDINNFLNWYQQKPGKAPKLLIYDASNLETGVPSRFSG |
| | SGSGTDFTFTISSLQPEDIATYYCQQYGNLPFTFGGGTKVEIKRAAAIEVM |
| | YPPPYLDNEKSNGTIIHVKGKHLCPSPLFPGPSKPFWVLVVVGGVLACYSL |
| | LVTVAFIIFWVRSKRSRLLHSDYMNMTPRRPGPTRKHYQPYAPPRDFAAY |
| | RSRVKFSRSADAPAYQQGQNQLYNELNLGRREEYDVLDKRRGRDPEMG |
| | GKPRRKNPQEGLYNELQKDKMAEAYSEIGMKGERRRGKGHDGLYQGLS |
| | TATKDTYDALHMQALPPR (SEQ ID NO. 66) |
| CLL-1 CAR 3 | QVQLQESGPGLVKPSETLSLTCTVSGGSISSYYWSWIRQPPGKGLEWIGYI |
| | YYSGSTNYNPSLKSRVTISVDTSKNQFSLKLSSVTAADTAVYYCVSLVYC |
| | GGDCYSGFDYWGQGTLVTVSSGGGGSGGGGSGGGGSDIQLTQSPSSLSAS |
| | VGDRVSFTCQASQDINNFLNWYQQKPGKAPKLLIYDASNLETGVPSRFSG |
| | SGSGTDFTFTISSLQPEDIATYYCQQYGNLPFTFGGGTKVEIKRAAALSNSI |
| | MYFSHFVPVFLPAKPTTTPAPRPPTPAPTIASQPLSLRPEACRPAAGGAVHT |
| | RGLDFACDIYIWAPLAGTCGVLLLSLVITLYCNHRNRSKRSRLLHSDYMN |
| | MTPRRPGPTRKHYQPYAPPRDFAAYRSRVKFSRSADAPAYQQGQNQLYN |
| | ELNLGRREEYDVLDKRRGRDPEMGGKPRRKNPQEGLYNELQKDKMAEA |
| | YSEIGMKGERRRGKGHDGLYQGLSTATKDTYDALHMQALPPR (SEQ ID |
| | NO: 67) |
| CLL-1 CAR 4 | DIQLTQSPSSLSASVGDRVSFTCQASQDINNFLNWYQQKPGKAPKLLIYDA |
| | SNLETGVPSRFSGSGSGTDFTFTISSLQPEDIATYYCQQYGNLPFTFGGGTK |
| | VEIKRGGGGSGGGGSGGGGSQVQLQESGPGLVKPSETLSLTCTVSGGSISS |
| | YYWSWIRQPPGKGLEWIGYIYYSGSTNYNPSLKSRVTISVDTSKNQFSLKL |
| | SSVTAADTAVYYCVSLVYCGGDCYSGFDYWGQGTLVTVSSAAALDNEK |
| | SNGTIIHVKGKHLCPSPLFPGPSKPFWVLVVVGGVLACYSLLVTVAFIIFW |
| | VRSKRSRLLHSDYMNMTPRRPGPTRKHYQPYAPPRDFAAYRSRVKFSRS |
| | ADAPAYQQGQNQLYNELNLGRREEYDVLDKRRGRDPEMGGKPRRKNPQ |
| | EGLYNELQKDKMAEAYSEIGMKGERRRGKGHDGLYQGLSTATKDTYDA |
| | LHMQALPPR (SEQ ID NO: 68) |
| CLL-1 CAR 5 | DIQLTQSPSSLSASVGDRVSFTCQASQDINNFLNWYQQKPGKAPKLLIYDA |
| | SNLETGVPSRFSGSGSGTDFTFTISSLQPEDIATYYCQQYGNLPFTFGGGTK |
| | VEIKRGGGGSGGGGSGGGGSQVQLQESGPGLVKPSETLSLTCTVSGGSISS |
| | YYWSWIRQPPGKGLEWIGYIYYSGSTNYNPSLKSRVTISVDTSKNQFSLKL |
| | SSVTAADTAVYYCVSLVYCGGDCYSGFDYWGQGTLVTVSSAAAIEVMY |
| | PPPYLDNEKSNGTIIHVKGKHLCPSPLFPGPSKPFWVLVVVGGVLACYSLL |
| | VTVAFIIFWVRSKRSRLLHSDYMNMTPRRPGPTRKHYQPYAPPRDFAAYR |
| | SRVKFSRSADAPAYQQGQNQLYNELNLGRREEYDVLDKRRGRDPEMGG |
| | KPRRKNPQEGLYNELQKDKMAEAYSEIGMKGERRRGKGHDGLYQGLST |
| | ATKDTYDALHMQALPPR (SEQ ID NO. 69) |
| CLL-1 CAR 6 | DIQLTQSPSSLSASVGDRVSFTCQASQDINNFLNWYQQKPGKAPKLLIYDA |
| | SNLETGVPSRFSGSGSGTDFTFTISSLQPEDIATYYCQQYGNLPFTFGGGTK |
| | VEIKRGGGGSGGGGSGGGGSQVQLQESGPGLVKPSETLSLTCTVSGGSISS |
| | YYWSWIRQPPGKGLEWIGYIYYSGSTNYNPSLKSRVTISVDTSKNQFSLKL |
| | SSVTADTAVYYCVSLVYCGGDCYSGFDYWGQGTLVTVSSAAALSNSIMY |
| | FSHFVPVFLPAKPTTTPAPRPPTPAPTIASQPLSLRPEACRPAAGGAVHTRG |
| | LDFACDIYIWAPLAGTCGVLLLSLVITLYCNHRNRSKRSRLLHSDYMNMT |
| | PRRPGPTRKHYQPYAPPRDFAAYRSRVKFSRSADAPAYQQGQNQLYNEL |
| | NLGREEYDVLDKRRGRDPEMGGKPRRKNPQEGLYNELQKDKMAEAYSE |
| | IGMKGERRRGKGHDGLYQGLSTATKDTYDALHMQALPPR (SEQ ID NO. |
| | 70) |
| CLL-1 CAR 7 | QVQLQESGPGLVKPSQTLSLTCTVSGGSISSGGFYWSWIRQHPGKGLEWI |
| | GYIHHSGSTHYNPSLKSRVTISIDTSKNLFSLRLSSVTAADTAVYYCASLV |
| | YCGGDCYSGFDYWGQGTLVTVSSGGGGSGGGGSGGGGSDIQLTQSPSSL |
| | SASVGDRVSFTCQASQDINNFLNWYQQKPGKAPKLLIYDASNLETGVPSR |
| | FSGSGSGTDFTFTISSLQPEDIATYYCQQYGNLPFTFGGGTKVEIKRAAALD |
| | NEKSNGTIIHVKGKHLCPSPLFPGPSKPFWVLVVVGGVLACYSLLVTVAFI |
| | IFWVRSKRSRLLHSDYMNMTPRRPGPTRKHYQPYAPPRDFAAYRSRVKFS |
| | RSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTY |
| | DALHMQALPPR (SEQ ID NO. 71) |
| CLL-1 CAR 8 | QVQLQESGPGLVKPSQTLSLTCTVSGGSISSGGFYWSWIRQHPGKGLEWI |
| | GYIHHSGSTHYNPSLKSRVTISIDTSKNLFSLRLSSVTAADTAVYYCASLV |
| | YCGGDCYSGFDYWGQGTLVTVSSGGGGSGGGGSGGGGSDIQLTQSPSSL |
| | SASVGDRVSFTCQASQDINNFLNWYQQKPGKAPKLLIYDASNLETGVPSR |
| | FSGSGSGTDFTFTISSLQPEDIATYYCQQYGNLPFTFGGGTKVEIKRAAAIE |
| | VMYPPPYLDNEKSNGTIIHVKGKHLCPSPLFPGPSKPFWVLVVVGGVLAC |
| | YSLLVTVAFIIFWVRSKRSRLLHSDYMNMTPRRPGPTRKHYQPYAPPRDF |
| | AAYRSRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQ |
| | GLSTATKDTYDALHMQALPPR (SEQ ID NO. 72) |
| CLL-1 CAR 9 | QVQLQESGPGLVKPSQTLSLTCTVSGGSISSGGFYWSWIRQHPGKGLEWI |
| | GYIHHSGSTHYNPSLKSRVTISIDTSKNLFSLRLSSVTAADTAVYYCASLV |
| | YCGGDCYSGFDYWGQGTLVTVSSGGGGSGGGGSGGGGSDIQLTQSPSSL |
| | SASVGDRVSFTCQASQDINNFLNWYQQKPGKAPKLLIYDASNLETGVPSR |
| | FSGSGSGTDFTFTISSLQPEDIATYYCQQYGNLPFTFGGGTKVEIKRAAALS |
| | NSIMYFSHFVPVFLPAKPTTTPAPRPPTPAPTIASQPLSLRPEACRPAAGGA |
| | VHTRGLDFACDIYIWAPLAGTCGVLLLSLVITLYCNHRNRSKRSRLLHSD |
| | YMNMTPRRPGPTRKHYQPYAPPRDFAAYRSRVKFSRSADAPAYQQGQN |
| | QLYNELNLGRREEYDVLDKRRGRDPEMGGKPRRKNPQEGLYNELQKDK |
| | MAEAYSEIGMKGERRRGKGHDGLYQGLSTATKDTYDALHMQALPPR |
| | (SEQ ID NO: 73) |
| CLL-1 CAR | QVQLVQSGAEVKKPGASVKVSCKVSGYTLTELSMHWVRQAPGKGLEW |
| 10 | MGGFDPEDGETIYAQKFQGRVTVTEDTSTDTAYMELSSLRSEDTAVYYC |
| | ATESRGIGWPYFDYWGQGTLVTVSSGGGGSGGGGSGGGGSDIQMTQSPS |
| | SLSASVGDRVTITCRASQSISSYLNWYQQKPGKAPKLLISGASSLKSGVPS |
| | RFSGSGSGTDFTLTISSLPPEDFATYYCQQSYSTPITFGQGTRLEIKRAAAL |
| | DNEKSNGTIIHVKGKHLCPSPLFPGPSKPFWVLVVVGGVLACYSLLVTVA |
| | FIIFWVRSKRSRLLHSDYMNMTPRRPGPTRKHYQPYAPPRDFAAYRSRVK |
| | FSRSADAPAYQQGQNQLYNELNLGRREEYDVLDKRRGRDPEMGGKPRR |
| | KNPQEGLYNELQKDKMAEAYSEIGMKGERRRGKGHDGLYQGLSTATKD |
| | TYDALHMQALPPR (SEQ ID NO: 74) |
| CLL-1 CAR | QVQLVQSGAEVKKPGASVKVSCKVSGYTLTELSMHWVRQAPGKGLEW |
| 11 | MGGFDPEDGETIYAQKFQGRVTVTEDTSTDTAYMELSSLRSEDTAVYYC |
| | ATESRGIGWPYFDYWGQGTLVTVSSGGGGSGGGGSGGGGSDIQMTQSPS |
| | SLSASVGDRVTITCRASQSISSYLNWYQQKPGKAPKLLISGASSLKSGVPS |
| | RFSGSGSGTDFTLTISSLPPEDFATYYCQQSYSTPITFGQGTRLEIKRAAAIE |
| | VMYPPPYLDNEKSNGTIIHVKGKHLCPSPLFPGPSKPFWVLVVVGGVLAC |
| | YSLLVTVAFIIFWVRSKRSRLLHSDYMNMTPRRPGPTRKHYQPYAPPRDF |
| | AAYRSRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQ |
| | GLSTATKDTYDALHMQALPPR (SEQ ID NO: 75) |
| CLL-1 CAR | QVQLVQSGAEVKKPGASVKVSCKVSGYTLTELSMHWVRQAPGKGLEW |
| 12 | MGGFDPEDGETIYAQKFQGRVTVTEDTSTDTAYMELSSLRSEDTAVYYC |
| | ATESRGIGWPYFDYWGQGTLVTVSSGGGGSGGGGSGGGGSDIQMTQSPS |
| | SLSASVGDRVTITCRASQSISSYLNWYQQKPGKAPKLLISGASSLKSGVPS |
| | RFSGSGSGTDFTLTISSLPPEDFATYYCQQSYSTPITFGQGTRLEIKRAAALS |
| | NSIMYFSHFVPVFLPAKPTTTPAPRPPTPAPTIASQPLSLRPEACRPAAGGA |
| | VHTRGLDFACDIYIWAPLAGTCGVLLLSLVITLYCNHRNRSKRSRLLHSD |
| | YMNMTPRRPGPTRKHYQPYAPPRDFAAYRSRVKFSRSADAPAYQQGQN |
| | QLYNELNLGRREEYDVLDKRRGRDPEMGGKPRRKNPQEGLYNELQKDK |
| | MAEAYSEIGMKGERRRGKGHDGLYQGLSTATKDTYDALHMQALPPR |
| | (SEQ ID NO: 76) |
| CLL-1 CAR | QVQLVESGGGVVQPGRSLRLSCAASGFTFSSYGMHWVRQAPGKGLEWV |
| 13 | AVISYDGSDKYYVDSVKGRFTISRDNSKNRLYLQMNSLRAEDTAVYYCA |
| | RERYSGRDYWGQGTLVTVSSGGGGSGGGGSGGGGSEIVMTQSPATLSVS |
| | PGERATLSCRASQSVSSLLTWYQQKPGQAPRLLIFGASTRATGIPARFSGS |
| | GSGTGFTLTISSLQSEDFAVYYCQQYDTWPFTFGPGTKVDFKRAAALDNE |
| | KSNGTIIHVKGKHLCPSPLFPGPSKPFWVLVVVGGVLACYSLLVTVAFIIF |
| | WVRSKRSRLLHSDYMNMTPRRPGPTRKHYQPYAPPRDFAAYRSRVKFSR |
| | SADAPAYQQGQNQLYNELNLGRREEYDVLDKRRGRDPEMGGKPRRKNP |
| | QEGLYNELQKDKMAEAYSEIGMKGERRRGKGHDGLYQGLSTATKDTYD |
| | ALHMQALPPR (SEQ ID NO: 78) |
| CLL-1 CAR | QVQLVESGGGVVQPGRSLRLSCAASGFTFSSYGMHWVRQAPGKGLEWV |
| 14 | AVISYDGSDKYYVDSVKGRFTISRDNSKNRLYLQMNSLRAEDTAVYYCA |
| | RERYSGRDYWGQGTLVTVSSGGGGSGGGGSGGGGSEIVMTQSPATLSVS |
| | PGERATLSCRASQSVSSLLTWYQQKPGQAPRLLIFGASTRATGIPARFSGS |
| | GSGTGFTLTISSLQSEDFAVYYCQQYDTWPFTFGPGTKVDFKRAAAIEVM |
| | YPPPYLDNEKSNGTIIHVKGKHLCPSPLFPGPSKPFWVLVVVGGVLACYSL |
| | LVTVAFIIFWVRSKRSRLLHSDYMNMTPRRPGPTRKHYQPYAPPRDFAAY |
| | RSRVKFSRSADAPAYQQGQNQLYNELNLGRREEYDVLDKRRGRDPEMG |
| | GKPRRKNPQEGLYNELQKDKMAEAYSEIGMKGERRRGKGHDGLYQGLS |
| | TATKDTYDALHMQALPPR (SEQ ID NO: 79) |
| CLL-1 CAR | QVQLVESGGGVVQPGRSLRLSCAASGFTFSSYGMHWVRQAPGKGLEWV |
| 15 | AVISYDGSDKYYVDSVKGRFTISRDNSKNRLYLQMNSLRAEDTAVYYCA |
| | RERYSGRDYWGQGTLVTVSSGGGGSGGGGSGGGGSEIVMTQSPATLSVS |
| | PGERATLSCRASQSVSSLLTWYQQKPGQAPRLLIFGASTRATGIPARFSGS |
| | GSGTGFTLTISSLQSEDFAVYYCQQYDTWPFTFGPGTKVDFKRAAALSNSI |
| | MYFSHFVPVFLPAKPTTTPAPRPPTPAPTIASQPLSLRPEACRPAAGGAVHT |
| | RGLDFACDIYIWAPLAGTCGVLLLSLVITLYCNHRNRSKRSRLLHSDYMN |
| | MTPRRPGPTRKHYQPYAPPRDFAAYRSRVKFSRSADAPAYQQGQNQLYN |
| | ELNLGRREEYDVLDKRRGRDPEMGGKPRRKNPQEGLYNELQKDKMAEA |
| | YSEIGMKGERRRG KGHDGLYQGLSTATKDTYDALHMQALPPR (SEQ ID |
| | NO: 80) |
| CLL-1 CAR | EIVMTQSPATLSVSPGERATLSCRASQSVSSLLTWYQQKPGQAPRLLIFGA |
| 16 | STRATGIPARFSGSGSGTGFTLTISSLQSEDFAVYYCQQYDTWPFTFGPGT |
| | KVDFKRGGGGSGGGGSGGGGSQVQLVESGGGVVQPGRSLRLSCAASGFT |
| | FSSYGMHWVRQAPGKGLEWVAVISYDGSDKYYVDSVKGRFTISRDNSKN |
| | RLYLQMNSLRAEDTAVYYCARERYSGRDYWGQGTLVTVSSAAALDNEK |
| | SNGTIIHVKGKHLCPSPLFPGPSKPFWVLVVVGGVLACYSLLVTVAFIIFW |
| | VRSKRSRLLHSDYMNMTPRRPGPTRKHYQPYAPPRDFAAYRSRVKFSRS |
| | ADAPAYQQGQNQLYNELNLGRREEYDVLDKRRGRDPEMGGKPRRKNPQ |
| | EGLYNELQKDKMAEAYSEIGMKGERRRGKGHDGLYQGLSTATKDTYDA |
| | LHMQALPPR (SEQ ID NO: 81) |
| CLL-1 CAR | EIVMTQSPATLSVSPGERATLSCRASQSVSSLLTWYQQKPGQAPRLLIFGA |
| 17 | STRATGIPARFSGSGSGTGFTLTISSLQSEDFAVYYCQQYDTWPFTFGPGT |
| | KVDFKRGGGGSGGGGSGGGGSQVQLVESGGGVVQPGRSLRLSCAASGFT |
| | FSSYGMHWVRQAPGKGLEWVAVISYDGSDKYYVDSVKGRFTISRDNSKN |
| | RLYLQMNSLRAEDTAVYYCARERYSGRDYWGQGTLVTVSSAAAIEVMY |
| | PPPYLDNEKSNGTIIHVKGKHLCPSPLFPGPSKPFWVLVVVGGVLACYSLL |
| | VTVAFIIFWVRSKRSRLLHSDYMNMTPRRPGPTRKHYQPYAPPRDFAAYR |
| | SRVKFSRSADAPAYQQGQNQLYNELNLGRREEYDVLDKRRGRDPEMGG |
| | KPRRKNPQEGLYNELQKDKMAEAYSEIGMKGERRRGKGHDGLYQGLST |
| | ATKDTYDALHMQALPPR (SEQ ID NO: 82) |
| CLL-1 CAR | EIVMTQSPATLSVSPGERATLSCRASQSVSSLLTWYQQKPGQAPRLLIFGA |
| 18 | STRATGIPARFSGSGSGTGFTLTISSLQSEDFAVYYCQQYDTWPFTFGPGT |
| | KVDFKRGGGGSGGGGSGGGGSQVQLVESGGGVVQPGRSLRLSCAASGFT |
| | FSSYGMHWVRQAPGKGLEWVAVISYDGSDKYYVDSVKGRFTISRDNSKN |
| | RLYLQMNSLRAEDTAVYYCARERYSGRDYWGQGTLVTVSSAAALSNSI |
| | MYFSHFVPVFLPAKPTTTPAPRPPTPAPTIASQPLSLRPEACRPAAGGAVHT |
| | RGLDFACDIYIWAPLAGTCGVLLLSLVITLYCNHRNRSKRSRLLHSDYMN |
| | MTPRRPGPTRKHYQPYAPPRDFAAYRSRVKFSRSADAPAYQQGQNQLYN |
| | ELNLGRREEYDVLDKRRGRDPEMGGKPRRKNPQEGLYNELQKDKMAEA |
| | YSEIGMKGERRRGKGHDGLYQGLSTATKDTYDALHMQALPPR (SEQ ID |
| | NO: 83) |

In some embodiments, the CAR of the present disclosure comprises an amino acid sequence that is at least about 75% identical, at least about 80% identical, at least about 85% identical, at least about 90% identical, at least about 95% identical, at least about 96% identical, at least about 97% identical, at least about 98% identical, at least about 99% identical, or 100% identical to any one of the amino acid sequences SEQ ID NOS:65-76 or 78-83.

In some embodiments, the CAR or a TCR of the present disclosure can further comprises a leader sequence or peptide (also referred to herein as a “signal peptide”). In certain embodiments, the leader peptide comprises an amino acid sequence that is at least about 75%, at least about 80%, at least about 85%, at least about 90%, at least about 95%, at least about 96%, at least about 97%, at least about 98%, at least about 99%, or 100% identical to the amino acid sequence MALPVTALLLPLALLLHAARP (SEQ ID NO: 96). In some embodiments, the leader peptide comprises the amino acid sequence of SEQ ID NO: 96.

The term “effector function” refers to a specialized function of the cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain may be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

In some embodiments, the cell can be engineered to express an exogenous T cell receptor (TCR). Libraries of TCRs may be screened for their selectivity to target antigens. In this manner, natural TCRs, which have a high avidity and reactivity toward target antigens may be selected, cloned, and subsequently introduced into a population of T cells used for adoptive immunotherapy.

In one embodiment described herein, T cells are modified by introducing a polynucleotide encoding subunit of a TCR that may form TCRs that confer specificity to T cells for tumor cells expressing a target antigen. In some embodiments, the subunits have one or more amino acid substitutions, deletions, insertions, or modifications compared to the naturally occurring subunit, so long as the subunits retain the ability to form TCRs conferring upon transfected T cells the ability to home to target cells, and participate in immunologically-relevant cytokine signaling. The TCRs may also bind target cells displaying the relevant tumor-associated peptide with high avidity, and optionally mediate efficient killing of target cells presenting the relevant peptide in vivo.

The nucleic acids encoding TCRs may be isolated from their natural context in a (naturally-occurring) chromosome of a T cell, and may be incorporated into suitable vectors as described elsewhere herein. Both the nucleic acids and the vectors comprising them may be transferred into a cell, which cell may be a T cell. The modified T cells are then able to express one or more chains of a TCR (and in some aspects two chains) encoded by the transduced nucleic acid or nucleic acids. In some embodiments, the TCR is an exogenous TCR because it is introduced into T cells that do not normally express the introduced TCR. An aspect of the TCRs is that it has high avidity for a tumor antigen presented by a major histocompatibility complex (MHC) or similar immunological component. In contrast to TCRs, CARs are engineered to bind target antigens in an MHC independent manner.

The protein encoded by the nucleic acids described herein may be expressed with additional polypeptides attached to the amino-terminal or carboxyl-terminal portion of the α-chain or the β-chain of a TCR so long as the attached additional polypeptide does not interfere with the ability of the α-chain or the β-chain to form a functional T cell receptor and the MHC dependent antigen recognition.

Antigens that are recognized by the TCRs contemplated herein include, but are not limited to cancer antigens, including antigens on both hematological cancers and solid tumors and viral induced cancers. Other illustrative antigens include, but are not limited to HPV oncoproteins, including HPV-16 E6 and HPV-16 E7, alpha folate receptor, 5T4, αᵥβ₆ integrin, BCMA, TACI, B7-H3, B7-H6, CAIX, CD19, CD20, CD22, CD28, CD30, CD33, CD44, CD44v6, CD44v7/8, CD70, CD79a, CD79b, CD123, CD137 (4-1BB), CD138, CD171, CEA, CSPG4, CLL-1, EGFR, EGFR family including ErbB2 (HERII), EGFRvIII, EGP2, EGP40, EPCAM, EphA2, EpCAM, FAP, fetal AchR, FRa, GD2, GD3, Glypican-3 (GPC3), HLA-Al+MAGEI, HLA-A2+MAGE1, HLAA3+MAGE1, HLA-Al+NY-ES0-1, HLA-A2+NY-ES0-1, HLA-A3+NY-ES0-1, IL-11Ra, IL-13Ra2, Lambda, Lewis-Y, Kappa, Mesothelin, Mucl, Muc16, NCAM, NKG2D Ligands, NY-ES0-1, PRAME, PSCA, PSMA, RORI, SSX, Survivin, TAG72, TEMs, and VEGFRII.

In some embodiments, the polynucleotide that encodes the CAR or TCR is introduced to the cell after the cell is engineered to reduce the MHC class I and/or II expression or activity. In some embodiments, the polynucleotide that encodes the CAR or TCR is introduced to the cell before the cell is engineered to reduce the MHC class I and/or II expression or activity. In some embodiments, the two rounds of engineering are carried out at least one day apart (not on the same day or within 24 hours).

### Treatments

The cells, e.g., allogeneic cells, of the present disclosure can be used for treating various diseases and conditions, in particular cancer. In one embodiment, the cancer may comprise Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In some embodiments, the cells of the present disclosure may be used to treat myeloid diseases including but not limited to acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN), or combinations thereof. Additional diseases include inflammatory and/or autoimmune diseases such as rheumatoid arthritis, psoriasis, allergies, asthma, Crohn's disease, IBD, IBS, fibromyalga, mastocytosis, lupus, and Celiac disease.

In some embodiments, the cells of the present disclosure may be used to treat cancer that arise from B cells, e.g., B-cell lymphomas. In some embodiments, cells of the present disclosure may be used to treat diffuse large B-cell lymphoma (DLBCL) not otherwise specified, primary mediastinal large B-cell lymphoma, high grade B-cell lymphoma, and DLBCL arising from follicular lymphoma.

Another embodiment described herein is a method of treating a cancer in a subject in need thereof comprising administering an effective amount, e.g., therapeutically effective amount of a composition comprising a cell of the present disclosure. The quantity and frequency of administration will be determined by such factors as the condition of the patient, and the type and severity of the patient's disease, although appropriate dosages may be determined by clinical trials. In some embodiments, the cancer is characterized with the expression of an antigen targeted by the CAR or TCR molecule, such as CD19 and/or CD20.

In some embodiments, the cancer is characterized with the expression of an antigen targeted by the CAR or TCR molecule, such as CLL-1.

In other embodiments, methods comprising administering a therapeutically effective amount of modified T cells contemplated herein or a composition comprising the same, to a patient in need thereof, alone or in combination with one or more therapeutic agents, are provided. In certain embodiments, the cells of the disclosure are used in the treatment of patients at risk for developing a cancer. Thus, the present disclosure provides methods for the treatment or prevention of a cancer comprising administering to a subject in need thereof, a therapeutically effective amount of the modified T cells of the disclosure.

It will be appreciated that target doses for CAR⁺/CAR-T⁺/TCR⁺ cells can range from about 1×10⁶ to about 2×10¹⁰ cells/kg (e.g., about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, about 9×10⁷ cells/kg, about 1×10⁸ cells/kg, about 2×10⁸ cells/kg, about 3×10⁸ cells/kg, about 4×10⁸ cells/kg, about 5×10⁸ cells/kg, about 6×10⁸ cells/kg, about 7×10⁸ cells/kg, about 8×10⁸ cells/kg, about 9×10⁸ cells/kg, about 1×10⁹ cells/kg, about 2×10⁹ cells/kg, about 3×10⁹ cells/kg, about 4×10⁹ cells/kg, about 5×10⁹ cells/kg, about 6×10⁹ cells/kg, about 7×10⁹ cells/kg, about 8×10⁹ cells/kg, about 9×10⁹ cells/kg, 1×10¹⁰ cells/kg, or about 2×10¹⁰ cells/kg). It will be appreciated that doses above and below this range may be appropriate for certain subjects.

One of ordinary skill in the art would recognize that multiple administrations of the compositions of the disclosure may be required to affect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

The methods for administering the cell compositions described herein includes any method which is effective to result in reintroduction of ex vivo genetically modified immune effector cells that either directly express an TCR or CAR in the subject or on reintroduction of the genetically modified progenitors of immune effector cells that on introduction into a subject differentiate into mature immune effector cells that express the TCR or CAR. One method comprises transducing peripheral blood T cells ex vivo with a nucleic acid construct in accordance with the present disclosure and returning the transduced cells into the subject.

Another embodiment described herein is a method of contacting a cancer cell with an engineered immune cell of the present disclosure, wherein the cancer cell growth is inhibited or reduced.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield similar results.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control. The contents of all references cited throughout this application are expressly incorporated herein by reference.

### EXAMPLES

### Example 1. Testing of RFX5 Knock-Out CAR T Cells

This example evaluated the in vitro efficacy of research grade zinc finger nucleases (ZFNs) targeting the deletion of the RFX5 (regulatory factor X5) and its ability to provide protection from T cell and NK cells mediated rejection for use in an allogeneic (MHC-mismatched) CAR-T cell product.

Methods: Healthy donor T cells were transduced with CD19/CD20 bicistronic CARs (with lentiviral vectors (LLV)) followed by electroporation of ZFNs targeting TRAC (T Cell Receptor Alpha Constant) and RFX5. Edited and non-edited CAR T cells were expanded for 14 days and were examined for expression of CAR, TCRαβ, MHC class I, and MHC class II expression on days 7 and 10. The majority of cells were frozen on day 10 for all functional and alloreactivity assays, while a small portion of cells were carried through to day 14 to confirm cell viability and expansion was maintained in edited cell conditions similar to unedited cells throughout manufacturing. CAR functionality was assessed by co-culture of CAR-T cells and CD19/CD20 positive tumor target cell lines (Raji WT) at various E:T ratios for cytotoxicity (24 and 48 hours), proliferation (120 hours), and cytokine secretion (24 hours). CAR-T persistence and cytotoxic function was assessed by serial stimulation of CAR-T cells with CD19/CD20 positive Raji MHC KO tumor cells at 1:1 E:T ratio every 3-4 days. Additionally, killing of edited and non-edited CAR T cells by MHC-mismatched CD8 T cells and NK cells was measured.

Results: RFX5 KO CAR-T cells had similar manufacturability and CAR functionality as 62 2M KO CAR-T cells. As shown in Tables 7A-C, RFX5 knockout (KO) allogeneic (Allo) CAR-T cells had good viability & similar expansion to B2M KO Allo CAR-T cells.

TABLE 7A
Raw Cell Counts (×10⁶)

| | | | LVV TRAC + | LVV TRAC + |
|---|---|---|---|---|
| Day | NTD | LVV | B2M KO | RFX5 KO |
| 3 | 9.9 | 9.9 | 9.9 | 9.9 |
| 5 | 44.4 | 41.6 | 9.8 | 10.2 |
| 7 | 172.15 | 163.6 | 5.5 | 34.01 |
| 10 | 544.4 | 651 | 244.5 | 244.5 |
| 12 | 1.7 | 2.4 | 4.4 | 3.6 |
| 14 | 1.9 | 11.2 | 19.5 | 15.8 |

TABLE 7B
Fold Expansion

| | | | LVV TRAC + | LVV TRAC + |
|---|---|---|---|---|
| Day | NTD | LVV | B2M KO | RFX5 KO |
| 3 | 1 | 1 | 1 | 1 |
| 5 | 4.48 | 4.20 | 0.99 | 1.03 |
| 7 | 17.39 | 16.53 | 0.56 | 3.44 |
| 10 | 54.99 | 65.76 | 24.70 | 24.70 |

TABLE 7C
Viability

| | | | LVV TRAC + | LVV TRAC + |
|---|---|---|---|---|
| Day | NTD | LVV | B2M KO | RFX5 KO |
| 0 | 96.1 | 96.1 | 96.1 | 96.1 |
| 3 | 89.7 | 88 | 88 | 88 |
| 5 | 88.1 | 89 | 86 | 89 |
| 7 | 93.3 | 93.2 | 90.2 | 91.5 |
| 10 | 98 | 92.1 | 95.8 | 95.6 |
| 12 | 96.3 | 94.3 | 89.2 | 91.2 |
| 14 | 97 | 91.2 | 95.6 | 94.3 |

Also, as shown in Table 8, the RFX5 or B2M KO did not affect the expression of the exogenous CAR molecules.

TABLE 8
CAR Expression

| CD19 CAR % | CD4/CD19 CAR+ | CD8/CD19 CAR+ |
|---|---|---|
| NTD | 0.022 | 0.031 |
| LVV | 88.6 | 82.2 |
| LVV TRAC + B2M KO | 83.9 | 68.8 |
| LVV TRAC + RFX5 KO | 85.7 | 64.8 |
| CD19 CAR MFI | CD4/CD19 CAR+ MFI | CD8/CD19 CAR+ MFI |
| LVV | 707 | 557 |
| LVV TRAC + B2M KO | 580 | 455 |
| LVV TRAC + RFX5 KO | 880 | 555 |
| CD20 CAR % | CD4/CD20 CAR+ | CD8/CD20 CAR+ |
| NTD | 0 | 0 |
| LVV | 91.8 | 88.9 |
| LVV TRAC + B2M KO | 88.7 | 78.8 |
| LVV TRAC + RFX5 KO | 88.1 | 72.1 |
| CD20 CAR MFI | CD4/CD20 CAR+ MFI | CD8/CD20 CAR+ MFI |
| LVV | 7461 | 6583 |
| LVV TRAC + B2M KO | 6497 | 4780 |
| LVV TRAC + RFX5 KO | 7649 | 4387 |

Tables 9A-C show that CD19/CD20 B2M KO & RFX5 KO CAR-Ts had similar activities.

TABLE 9A
Serial Killing - Raji MHC I/II KO line

| | | | | LVV TRAC + B2M | | | LVV TRAC + RFX5 | | |
|---|---|---|---|---|---|---|---|---|---|
| Round | LVV | | | KO | | | KO | | |
| 1 | 65.69 | 64.76 | 61.46 | 72.92 | 76.29 | 74.80 | 67.96 | 65.05 | 71.57 |
| 2 | 27.72 | 27.72 | 28.40 | 73.39 | 70.41 | 73.64 | 81.88 | 76.21 | 69.51 |
| 3 | 0 | 0 | 0 | 11.18 | 12.75 | 4.73 | −1.38 | 25.03 | 25.79 |
| 4 | 0 | 0 | 0 | 3.86 | 4.69 | −2.87 | 8.68 | 14.17 | 10.16 |

TABLE 9B
Cytokine Production (pg/mL) - 24 hour, Raji WT line

| | NTD | | | LVV | | | LVV TRAC + B2M KO | | |
|---|---|---|---|---|---|---|---|---|---|
| IFNγ | 551.1 | 753.8 | 485.1 | 214065 | 176671 | 177473 | 142278 | 144393 | 150414 |
| IL-2 | 45.9 | 61.6 | 51.8 | 3998 | 3699.6 | 3496.5 | 11345.7 | 11630.8 | 12364.3 |
| TNFα | 9.4 | 9.8 | 9.5 | 1035.2 | 852.7 | 842.9 | 1431.3 | 1420.3 | 1367.7 |

| | LVV TRAC + RFX5 KO | | |
|---|---|---|---|
| IFNγ | 129964.4 | 89862.3 | 93004.4 |
| IL-2 | 12061.9 | 11343.9 | 12480.2 |
| TNFα | 1471.4 | 1327.9 | 1483.1 |

TABLE 9C
Cytotoxicity - 48 hour, Raji WT cell line

| E:T | | | | | | | LVV TRAC + B2M | | |
|---|---|---|---|---|---|---|---|---|---|
| RATIO: | NTD | | | LVV | | | KO | | |
| 1:1 | −29.97 | −26.23 | −20.40 | 98.39 | 97.01 | 97.92 | 94.47 | 90.29 | 89.78 |
| 1:3 | −11.48 | −9.09 | 3.94 | 77.91 | 70.96 | 72.92 | 51.53 | 51.60 | 51.89 |
| 1:9 | −19.16 | −5.43 | −10.04 | 42.99 | 35.88 | 40.60 | 27.87 | 30.67 | 23.84 |

| E:T | LVV TRAC + RFX5 | | |
|---|---|---|---|
| RATIO: | KO | | |
| 1:1 | 93.47 | 88.24 | 95.19 |
| 1:3 | 52.22 | 48.35 | 53.96 |
| 1:9 | 24.45 | 24.42 | 24.03 |

RFX5 KO CAR T cells had decreased HLA Class I expression with a similar MFI as β2M KO CAR T cells, which reduced host alloreactivity response (Table 10). The table shows that RFX5 KO resulted in MHC Class I knockdown (down-regulation), While B2M KO resulted in MHC Class I knockout (elimination). Meanwhile, similar TCR KO was observed in both TRAC+B2M KO & TRAC+RFX5 KO cells. MHC Class I MFI was similar for both Class I knockout and knockdown cells. Efficient MHC Class II KO, however, was only observed in TRAC+RFX5 KO cells.

TABLE 10
TCR and MHC molecule expression

| TCRab | CD4 | CD8 |
|---|---|---|
| NTD | 98.7 | 97.6 |
| LVV | 93.2 | 90.6 |
| LVV TRAC + B2M KO | 1.71 | 1.96 |
| LVV TRAC + RFX5 KO | 1.6 | 2.36 |
| Class I | | |
| NTD | 99.7 | 100 |
| LVV | 99.9 | 100 |
| LVV TRAC + B2M KO | 10.3 | 10.6 |
| LVV TRAC + RFX5 KO | 56.9 | 63.5 |
| Class II | | |
| NTD | 72.2 | 89.1 |
| LVV | 95.5 | 99.2 |
| LVV TRAC + B2M KO | 93.5 | 97.6 |
| LVV TRAC + RFX5 KO | 14 | 22 |

| Class I | | | LVV TRAC + | LVV TRAC + |
|---|---|---|---|---|
| MFI+ | NTD | LVV | B2M KO | RFX5 KO |
| Day 10 | 3845 | 6372 | 2455 | 1546 |

Knocking out RFX5 provided enhanced protection from host NK killing compared to knocking out β2M as well as reduced rejection from mismatched host CD8 T cells compared to non-edited CAR-T cells (Tables 11A-B). Table 11A-B shows that RFX5 KO provided protection from host NKs and reduced CD8 rejection. Table 11A: RFX5 KO improved NK protection compared to B2M KO in 3 out of 3 donors; Table 11B: RFX5 KO reduced CD8 rejection to a similar level as B2M KO in ⅓ mismatched donors, and reduced CD8 rejection compared to unedited cells in ⅔ mismatched donors. RFX5 KO cells exhibited an absence of MHC class II expression (Table 10), which is predicted to minimize responses by host CD4 T cells.

TABLE 11A
NK Killing

| NK Assay | NK Donor 1 | | | NK Donor 2 | | | NK Donor 3 | | |
|---|---|---|---|---|---|---|---|---|---|
| NTD | 26.3 | 29 | | 31.1 | 33.1 | 28.7 | 29.1 | 30.5 | 30.8 |
| LVV | 32.3 | 31.6 | 33.1 | 37.6 | 34.4 | 36.7 | 40.2 | 34.9 | 37.5 |
| LVV | 67.1 | 61.9 | 71.2 | 70.7 | 72.5 | 72.8 | 84.3 | 85.5 | 86.5 |
| TRAC + | | | | | | | | | |
| B2M KO | | | | | | | | | |
| LVV | 43.8 | 36.7 | 40.3 | 53.8 | 52.8 | 50 | 48 | 51 | 51.3 |
| TRAC + | | | | | | | | | |
| RFX5 KO | | | | | | | | | |

TABLE 11B
CD8 Killing

| CD8 | | | | | | | LVV TRAC + | | | LVV TRAC + | | |
|---|---|---|---|---|---|---|---|---|---|---|---|---|
| Assay | NTD | | | LVV | | | B2M KO | | | RFX5 KO | | |
| CD8 | 90.5 | 97 | 98.1 | 94.2 | 87.5 | 91.3 | 28.7 | 26.4 | 28.3 | 56.6 | 51.6 | 69.2 |
| Donor 1 | | | | | | | | | | | | |
| CD8 | 93.6 | 93.5 | 93.5 | 80.8 | 80.7 | 88.4 | 18.1 | 24.5 | 21.5 | 53.1 | 45.6 | 64.3 |
| Donor 2 | | | | | | | | | | | | |
| CD8 | 78.2 | 73.3 | 76.6 | 43.2 | 41.4 | 50.9 | 23.4 | 26.5 | 23.4 | 28.9 | 30.1 | 33.7 |
| Donor 3 | | | | | | | | | | | | |

This example demonstrated that the deletion of the transcription factor, RFX5, allowed generation of CAR-T cells with low levels of MHC class I and an absence of MHC class II expression that led to reduced killing by MHC-mismatched CD8 T cells and NK cells.

### Example 2. Clinical Testing of Allogeneic TRAC-RFX5 Anti-CD19/CD20 CAR

Healthy donor-derived allogeneic (HD Allo) products directed to CD19 and CD20 will be developed for the treatment of cancer irrespective of patients' HLA type, utilizing zinc finger nucleases (ZFNs) to knock out genes. The product will comprise of healthy donor-derived T cells that are directed to CD19 and CD20. Using ZFNs, the TRAC gene will be disrupted to address risks of graft-versus-host disease (GVHD) and the RFX5 gene will be disrupted to reduce expression of MHC class I and II to minimize host rejection.

The TRAC and RFX5 genes can alternatively be disrupted by any of the gene editing approaches described herein or known in the art (e.g., via sgRNAs and CRISPR/cas9).

All modifications are designed to achieve the following essential product attributes: (i) comparable or improved in vivo activity profile relative to allogeneic anti-CD19/CD20 CAR with disruption of the TRAC and B2M loci (allogeneic TRAC-B2M CD19/CD20), and conventional, non-gene edited anti-CD19 CAR T cells, (ii) minimal to zero risk of GVHD, and (iii) minimal risk of rejection by the patient's endogenous CD4 and CD8 T cells and NK cells as demonstrated using in vitro assays. The following objectives are anticipated to be achieved.
comparable or improved in vivo activity relative to allogeneic TRAC/B2M KO CD19/CD20 CAR-T cells as evidenced by tumor cell clearance;
comparable or improved CAR expression and cell expansion kinetics (in vivo and during manufacturing) compared to allogeneic TRAC-B2M KO CD19/CD20 CAR-T cells;
reduced mismatched NK cell rejection in vitro relative to allogeneic TRAC-B2M KO CD19/CD20 CAR-T cells as evidenced by reduced NK cell killing of allogeneic CART cells; and
reduced mismatched CD4 T cell recognition and CD8 T cell rejection in vitro relative to nonedited anti-CD19 or anti-CD19/20 CAR-T cells as evidenced by reduced killing and/or cytokine secretion.

### Example 3. Testing of RFX5 Knock-Out CAR T Cells

This example evaluated the in vitro efficacy of research grade Clustered Regularly Interspaced Palindromic Repeats (CRISPR) and the CRISPR associated protein 9 (Cas9) (CRISPR/Cas9) ribonucleoprotein targeting the deletion of the RFX5 gene and its ability to provide protection from T cell and NK cells mediated rejection for use in an allogeneic (MHC-mismatched) CAR-T cell product.

Methods: Healthy donor T cells were transduced with a CLL-1 CAR (with lentiviral vectors (LLV)) followed by electroporation of CRISPR/Cas9 ribonucleoproteins with single guide RNAs (sgRNAs) targeting TRAC (T Cell Receptor Alpha Constant) and B2M (beta-2-microglobulin) or targeting TRAC and RFX5. β2M knockout (KO) allogeneic (Allo) CAR-T cells (i.e., TRAC+B2M KO, where both TRAC and B2M were knocked out), RFX5 KO Allo CAR-T cells (i.e., TRAC+RFX5 KO, where both TRAC and RFX5 were knocked out), non-edited CAR-T cells (i.e., LVV) and non-transduced control cells (i.e., NTD) were expanded for 10 days and were examined for expression of CAR, TCRαβ, MHC class I, and MHC class II expression on day 10. The cells were frozen on day 10 for all functional and alloreactivity assays. Killing of edited and non-edited CAR T cells by MHC-mismatched NK cells was measured.

Results: RFX5 KO CAR-T cells had similar manufacturability and CAR functionality as β2M KO CAR-T cells. As shown in Tables 12A-C, RFX5 knockout (KO) allogeneic (Allo) CAR-T cells had good viability and similar expansion to B2M KO Allo CAR-T cells.

TABLE 12A
Raw Cell Counts (×10⁶)

| | | | LVV TRAC + | LVV TRAC + |
|---|---|---|---|---|
| Day | NTD | LVV | B2M KO | RFX5 KO |
| 3 | 9 | 8 | 8 | 8 |
| 5 | 54 | 47 | 37 | 40 |
| 7 | 258 | 239 | 205 | 227 |
| 10 | 876 | 712 | 720 | 828 |

TABLE 12B
Fold Expansion

| | | | LVV TRAC + | LVV TRAC + |
|---|---|---|---|---|
| Day | NTD | LVV | B2M KO | RFX5 KO |
| 3 | 1 | 1 | 1 | 1 |
| 5 | 5.95 | 6.15 | 4.82 | 5.15 |
| 7 | 28.68 | 31.22 | 26.61 | 29.48 |
| 10 | 97.33 | 93.14 | 93.49 | 107.53 |

TABLE 12C
Viability

| | | | LVV TRAC + | LVV TRAC + |
|---|---|---|---|---|
| Day | NTD | LVV | B2M KO | RFX5 KO |
| 0 | 93.0 | | | |
| 3 | 93.2 | 93.5 | 90.0 | 90.5 |
| 5 | 94.5 | 93.6 | 95.0 | 95.9 |
| 7 | 96.8 | 96.1 | 96.4 | 96.7 |
| 10 | 96.2 | 89.9 | 90.7 | 91.9 |

Also, as shown in Table 13, the RFX5 or B2M KO did not affect the expression of the exogenous CAR molecules.

TABLE 13
CAR Expression at Day 10

| CLL1 CAR % | CD4/CLL1 CAR+ | CD8/CLL1 CAR+ |
|---|---|---|
| NTD | 0.032 | 0.033 |
| LVV | 90.8 | 92.5 |
| LVV TRAC + B2M KO | 91.1 | 92.8 |
| LVV TRAC + RFX5 KO | 91.9 | 93.7 |

Table 14 shows that RFX5 KO resulted in MHC Class I knockdown (down-regulation). Meanwhile, similar TCR KO was observed in both TRAC+B2M KO and TRAC+RFX5 KO cells. Efficient MHC Class II KO, however, was only observed in TRAC+RFX5 KO cells.

TABLE 14
TCR and MHC molecule expression at Day 10

| TCRab | CD4 | CD8 |
|---|---|---|
| NTD | 90.9 | 86.9 |
| LVV | 95.3 | 92.7 |
| LVV TRAC + B2M KO | 1.45 | 2.03 |
| LVV TRAC + RFX5 KO | 1.51 | 2.24 |
| Class I | | |
| NTD | 98.81 | 99.31 |
| LVV | 98.67 | 99 |
| LVV TRAC + B2M KO | 16.19 | 16.2 |
| LVV TRAC + RFX5 KO | 66.44 | 53.5 |
| Class II | | |
| NTD | 67.2 | 79.2 |
| LVV | 67.3 | 93.4 |
| LVV TRAC + B2M KO | 63.6 | 90.5 |
| LVV TRAC + RFX5 KO | 12.4 | 18.8 |

Knocking out RFX5 provided enhanced protection from host NK killing compared to knocking out β2M (Table 15). Table 15: RFX5 KO improved NK protection compared to B2M KO in 3 out of 3 donors.

TABLE 15
NK Killing (% Death)

| NK Assay | NK Donor 1 | | | NK Donor 2 | | | NK Donor 3 | | |
|---|---|---|---|---|---|---|---|---|---|
| NTD | 17.5 | 17 | 15.9 | 22.6 | 20.6 | 21.2 | 14.9 | 14.8 | 15 |
| LVV | 35.3 | 31.7 | 34.7 | 41.4 | 40.7 | 38.4 | 30.9 | 33.1 | 32 |
| LVV TRAC + | 70.9 | 71.4 | 68.2 | 74.3 | 70.8 | 73.4 | 61.8 | 62.8 | 65.6 |
| B2M KO | | | | | | | | | |
| LVV TRAC + | 34.9 | 35.8 | 37.1 | 44.1 | 41.1 | 42.4 | 36.5 | 33.7 | 33 |
| RFX5 KO | | | | | | | | | |

This example demonstrated that the deletion of the transcription factor, RFX5, allowed generation of CAR-T cells with low levels of MHC class I and an absence of MHC class II expression that led to reduced killing by NK cells as compared to B2M KO cells.

### Example 4. Testing of RFX5 Knock-Out CAR T Cells

This Example will evaluate the in vivo efficacy of RFX5 KO CAR T cells in mouse models for B-cell lymphoma and acute myeloid leukemia.

Methods: RFX5 KO CAR-T cells and corresponding controls generated by the methods described in Examples 1 and 3 will be used for in vivo studies. 6-7-week-old, female NSG mice, 5 mice/group, will be evaluated. Two doses of CAR+ T cells will be tested: 1×10⁶ or 2×10⁶ CAR+ T cells/mouse and 5×10⁶ or 10×10⁶ CAR+ T cells/mouse. Tumor cells expressing relevant antigen will be implanted intravenously via the lateral tail vein into 6-7-week-old female NSG mice.

After tumor implantation, the following groups of B-cell lymphoma or AML tumor bearing mice will be evaluated: (1) no treatment; (2) treatment with non-transduced (NTD) T cells; (3) treatment with non-edited CAR-T cells; (4) treatment with allogeneic RFX5 and TRAC KO CAR-T cells; and (5) treatment with allogeneic B2M and TRAC KO CAR-T cells.

Tumor killing will be assessed by assessing the effects of T-cell products on tumor burden by bioluminescence (bioluminescence correlates positively with tumor burden) and animal survival. RFX5 KO CAR-T cell expansion kinetics and persistence will be assessed by ex-vivo analysis of blood samples taken 24 hours after CAR-T cell infusion followed by weekly sampling to track CAR expression, TCR, MHC Class I, MHC Class II, and other relevant T cell phenotyping markers.

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. An isolated human immune cell engineered to have (i) MHC class I activity or expression that is from 10% to 80% lower as compared to a reference cell; (ii) MHC class II activity or expression that is at least 75% lower as compared to a reference cell; and (iii) an exogenous polynucleotide encoding a chimeric antigen receptor (CAR) or a T-cell receptor (TCR).

2. The immune cell of claim 1, which is a T cell or a NK cell.

3. The immune cell of claim 1, wherein an endogenous gene of RFX5 (regulatory factor X5), TAP1 (Transporter associated with antigen processing 1), TAP2 (Antigen peptide transporter 2), TRAC (T Cell Receptor Alpha Constant) and/or CIITA (class II, major histocompatibility complex, transactivator) is inactivated or deficient.

4. (canceled)

5. The immune cell of claim 1, wherein an endogenous gene of RFX5 is inactivated or deficient.

6. (canceled)

7. (canceled)

8. The immune cell of claim 1, wherein the endogenous gene of B2M (Beta-2-microglobulin) is not engineered or has normal activity of B2M.

9. The immune cell of claim 1, wherein the CAR or the TCR recognizes CD19 and/or CD20 or CLL-1.

10. (canceled)

11. The immune cell of claim 9, wherein the CAR comprises the amino acid sequence of SEQ ID NO:26-27, 65-76 or 78-83 or a sequence having 90% sequence identity to SEQ ID NO:26-27, 65-76 or 78-83.

12. (canceled)

13. (canceled)

14. The immune cell of claim 1, wherein the immune cell is edited by CRISPR/Cas9, a zinc finger nuclease (ZFN), a TALEN, a MegaTAL, a meganuclease, Cpf1, homologous recombination, a single stranded oligodeoxynucleotide (ssODN), or base editing.

15. The immune cell of claim 1, wherein the reference immune cell has not been edited to have reduced MHC class I expression or reduced MHC class II expression.

16. A method for preparing an allogeneic cell with reduced activity in inducing graft-versus-host disease (GVHD) or host rejection, comprising engineering a cell to (i) have reduced expression or activity of MHC class I by 10% to 80% as compared to a reference cell, (ii) have reduced expression or activity of MHC class II by at least 75% as compared to a reference cell, and (iii) have an exogenous polynucleotide encoding a chimeric antigen receptor (CAR) or a T-cell receptor (TCR).

17. The method of claim 16, which reduces, in the allogeneic cell, the expression or activity of RFX5 (regulatory factor X5), TAP1 (Transporter associated with antigen processing 1), TAP2 (Antigen peptide transporter 2), TRAC (T Cell Receptor Alpha Constant) and/or CIITA (class II, major histocompatibility complex, transactivator).

18-21. (canceled)

22. The method of claim 16, wherein an endogenous gene of B2M (Beta-2-microglobulin) in the cell is not engineered.

23. (canceled)

24. The method of claim 16, wherein the allogeneic cell is edited by CRISPR/Cas9, a zinc finger nuclease (ZFN), a TALEN, a MegaTAL, a meganuclease, Cpf1, homologous recombination,a single stranded oligodeoxynucleotide (ssODN), or base editing.

25. The method of claim 16, wherein the allogeneic cell is a T cell or a NK cell.

26. (canceled)

27. The method of claim 16, wherein the CAR or the TCR recognizes CD19 and/or CD20 or CLL-1.

28. The method of claim 27, wherein the CAR comprises the amino acid sequence of SEQ ID NOS:26-27, 65-76 or 78-83 or an amino acid sequence having 90% identity to SEQ ID NO:26-27, 65-76 or 78-83.

29. (canceled)

30. (canceled)

31. The method of of claim 16, wherein the CAR or TCR is introduced to the cell prior to an editing of a gene.

32. A method for treating cancer in a patient in need thereof, comprising administering to the patient the immune cell of claim 1.

33. (canceled)

34. The method of claim 32, wherein the cancer is lymphoma, leukemia, myeloma, acute lymphoblastic leukemia, acute myelogenous leukemia, chronic lymphocytic leukemia, chronic myelogenous leukemia, Hodgkin's lymphoma, or non-Hodgkin's lymphoma.

35. A method of contacting a cancer cell with the immune cell of claim 1, wherein the cancer cell growth is inhibited or reduced.


SEQUENCE LISTING (96 sequences)

SEQ ID NO: 1 - protein, 120 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARGGGSWYSNWFDPWGQGTMVTVSS

SEQ ID NO: 2 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQDRSLPPTFGGGTKVEIK

SEQ ID NO: 3 - protein, 121 aa, synthetic construct
QVQLQQWGAGLLKPSETLSLTCAVYGGSFSGIHWNWIRQPPGKGLEWIGDIDTSGSTNYNPSLKSRVTISVDTSKNQFSLKLSSVTAADTAVYYCARLGQESATYLGMDVWGQGTTVTVSS

SEQ ID NO: 4 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYTYPFTFGGGTKVEIK

SEQ ID NO: 5 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSS

SEQ ID NO: 6 - protein, 107 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIK

SEQ ID NO: 7 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 8 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 9 - protein, 124 aa, synthetic construct
QVQLQESGPGLVKPSETLSLTCTVSGGSISSPDHYWGWIRQPPGKGLEWIGSIYASGSTFYNPSLKSRVTISVDTSKNQFSLKLSSVTAADTAVYYCARETDYSSGMGYGMDVWGQGTTVTVSS

SEQ ID NO: 10 - protein, 107 aa, synthetic construct; identical to SEQ ID NO: 6

SEQ ID NO: 11 - protein, 123 aa, synthetic construct
QITLKESGPTLVKPTQTLTLTCTFSGFSLDTEGVGVGWIRQPPGKALEWLALIYFNDQKRYSPSLKSRLTITKDTSKNQVVLTMTNMDPVDTAVYYCARDTGYSRWYYGMDVWGQGTTVTVSS

SEQ ID NO: 12 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAYAYPITFGGGTKVEIK

SEQ ID NO: 13 - protein, 127 aa, synthetic construct
QVQLQQWGAGLLKPSETLSLTCAVYGGSFEKYYWSWIRQPPGKGLEWIGEIYHSGLTNYNPSLKSRVTISVDTSKNQFSLKLSSVTAADTAVYYCARVRYDSSDSYYYSYDYGMDVWGQGTTVTVSS

SEQ ID NO: 14 - protein, 113 aa, synthetic construct
DIVLTQSPDSLAVSLGERATINCKSSQSVLYSSNNKNYLAWYQQKPGQPPKLLIYWASSRESGVPDRFSGSGSGTDFTLTISSLQAEDVAVYYCQQSYSFPWTFGGGTKVEIK

SEQ ID NO: 15 - protein, 127 aa, synthetic construct
QVQLQQWGAGLLKPSETLSLTCAVYGGSFSRYVWSWIRQPPGKGLEWIGEIDSSGKTNYNPSLKSRVTISVDTSKNQFSLKLSSVTAADTAVYYCARVRYDSSDSYYYSYDYGMDVWGQGTTVTVSS

SEQ ID NO: 16 - protein, 113 aa, synthetic construct; identical to SEQ ID NO: 14

SEQ ID NO: 17 - protein, 127 aa, synthetic construct
QVQLQQWGAGLLKPSETLSLTCAVYGGSFSGYAWSWIRQPPGKGLEWIGEIDHRGFTNYNPSLKSRVTISVDTSKNQFSLKLSSVTAADTAVYYCARVRYDSSDSYYYSYDYGMDVWGQGTTVTVSS

SEQ ID NO: 18 - protein, 113 aa, synthetic construct; identical to SEQ ID NO: 14

SEQ ID NO: 19 - protein, 123 aa, synthetic construct
QVQLQQWGAGLLKPSETLSLTCAVYGGSFQKYYWSWIRQPPGKGLEWIGEIDTSGFTNYNPSLKSRVTISVDTSKNQFSLKLSSVTAADTAVYYCARVGRYSYGYYITAFDIWGQGTTVTVSS

SEQ ID NO: 20 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQHYSFPFTFGGGTKVEIK

SEQ ID NO: 21 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 23 - protein, 120 aa, synthetic construct
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 24 - protein, 107 aa, synthetic construct
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 25 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 1018 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGEGRGSLLTCGDVEENPGPMALPVTALLLPLALLLHAARPQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGSTSGSGKPGSGEGSTKGDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 733 aa, synthetic construct
MLLLVTSLLLCELPHPAFLLIPDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKGGGGSGKPGSGEGGSQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGGGGSGKPGSDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 3057 bp, synthetic construct
  source 1..3057: mol_type = other DNA
atgctgctgctggtgacatctctgctgctttgcgagctgccccaccctgccttcctgcttatccccgacattcagatgacccagaccaccagcagcctgagcgccagcttaggagatagagttaccatcagctgcagagccagccaggacatcagcaaatacctgaactggtatcagcagaagcccgacggcactgtgaaactgcttatttaccacacctccagactgcacagcggcgttcccagcagattctctggcagcggatctggaaccgactacagcctcaccatctccaacctggagcaggaggacatcgccacctacttctgccagcagggcaacacactgccctacaccttcggaggaggaaccaagctggagatcaccgggggaggaggctctggaggcggcggatcaggaggagggggatctgaggttaagctgcaggagagcggccctggcctggtggctcctagccaatctttatctgtgacctgcactgtgtccggcgttagcctgcccgattatggcgtttcctggatcagacagccccccagaaagggcctggaatggctgggcgttatctggggcagcgagaccacatactacaacagcgccctgaagagcagacttacgattatcaaggacaacagcaagagccaggttttcctgaagatgaacagcctgcagaccgacgacaccgccatctactactgcgctaagcactactactacggcggcagctacgccatggactactggggccagggaacaagcgttaccgttagcagcgctgctgcactggacaacgagaagagcaacggcaccatcatccacgttaagggcaagcacctgtgccccagccctctgttccctggaccttctaagcctttctgggttctggtggtggtcggcggcgttttagcctgttacagccttctggtgactgtggccttcatcatcttttgggttagaagcaagagaagcagactgctccacagcgactacatgaacatgacccccagacggcctggccccaccagaaagcattaccagccctacgctcctcccagagacttcgccgcctacaggagcagagttaaattcagcagatccgccgatgcccccgcttaccaacagggacaaaaccagctgtacaatgagctcaacctggggagaagagaagaatacgacgttctggataagagaaggggcagagatcccgaaatggggggcaagcccagacgcaagaaccctcaggaggggctttacaacgaactgcagaaggataagatggctgaggcttactcggagattgggatgaagggggagagaaggcggggcaagggacacgatggcttataccaggggctgagcaccgccaccaaggacacatacgacgctcttcatatgcaggctctgcccccaagaagggctaagagatctggctctggcgagggcagaggcagcttgcttacatgtggcgatgtggaggagaaccccgggcccatggctcttcctgtgacagctcttctgctgcccctggccctgcttctgcatgctgctagacctcagcttcagctccaagagagcggacctggcttagtgaagcccagcgaaaccctgtccctcacctgcaccgtttctggcggaagcatcagcagctccagctattactggggatggatcaggcagccccctggcaagggtttagaatggatcggctcgatatattactccggcagcacctactataaccccagcttgaagagccgggttaccatttctgtggacacatcaaagaaccagttcagcctgaagctgagctctgtgactgccgccgacacagctgtgtactactgtgccagagagacagactactccagcggcatgggctacggcatggatgtgtggggacaaggaaccaccgttactgtgagcagcggttccaccagcggctcaggcaagcctggctcaggagaaggaagcaccaagggggatatacagatgacacagagcccctccagcctgtccgccagcgttggcgatcgtgtaacgatcacctgccgggcctctcagagcatcaactcctacctcaattggtatcaacagaagccaggcaaggcccccaaattactcatctacgccgccagcagcttacagagcggggttccctctagattctccggctccggttctggaacagatttcaccctcactatctccagcttgcagcccgaggatttcgccacttattactgtcagcagagcctggccgaccccttcacattcggcggaggcacaaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatga

SEQ ID NO: 29 - DNA, 2202 bp, synthetic construct
  source 1..2202: mol_type = other DNA
atgcttctcctggtgacaagccttctgctctgtgagttaccacacccagcattcctcctgatcccagacatccagatgacccagtctccatcctccctgtctgcatctgtaggagacagagtcaccatcacttgccgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgccagcaaagcctcgccgaccctttcacttttggcggagggaccaaggttgagatcaaaggggggggtggaagtgggaagcctggcagcggcgagggcggcagtcagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagttctgtgaccgccgcagacacggcggtgtactactgcgccagagagactgactacagcagcggaatgggatacggaatggacgtatggggccagggaacaactgtcaccgtctcctcaggcggtggcggcagtgggaagcctggcagcgatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggggggggtggaagtgggggcggtggcagcggcggtggcggcagtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagccgctgccctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgatga

SEQ ID NO: 30 - protein, 467 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 813 bp, synthetic construct
  source 1..813: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggctattactggagctggatccggcagcctcctggaaaaggattagaatggatcggcgagatagaccacagcgggagcacaaactacaaccccagcctgaaatcgcgggttacaatctctgtggacacaagcaagaatcagttctccctgaagctgagcagcgttactgccgccgacacagctgtgtactattgcgccagaggcggaggctcctggtacagcaactggttcgatccttggggccaaggcaccatggtgaccgtttccagcggctctacaagcggcagcgggaaacctggttctggagagggcagcacaaagggcgacatccagatgacacagagccccagcacccttagcgcctctgtgggagatagggttaccattacctgcagggcttcccagagcatcagcagctggctggcatggtatcaacagaagcctggcaaggctcccaagctgctcatctatgacgcctccagcctggaaagcggggttccctccagatttagcggctcaggctccggaacagagttcacccttaccatctctagcctgcaacccgacgacttcgctacttattactgtcaacaagacagaagcttgccccccacattcggcggagggaccaaggttgagatcaag

SEQ ID NO: 32 - DNA, 834 bp, synthetic construct
  source 1..834: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggcatccactggaactggatccggcagcctcctggcaaaggccttgaatggatcggcgatatcgacaccagcggctccaccaactacaaccccagcctgaaatcgagggttacaatctctgtggacacaagcaagaatcagttctccctgaagctgagcagcgttactgccgccgacacagctgtgtactattgcgccagactgggccaggaaagcgctacctaccttggcatggatgtgtgggggcagggcaccaccgttactgttagctctggctcaacaagcggcagcggcaagcctggctcaggagaaggaagcacaaagggcgacattgtaatgactcagagccccgacagcctggccgttagcttaggcgaaagggctacaatcaattgcaagagcagccagagcgttctgtacagcagcaacaacaagaactacctcgcatggtatcaacagaagccaggccagcctcccaagctgctcatctactgggcttccaccagagagagcggggttcccgatagattctccggctccggttctggaacagatttcacgctcacaatcagcagcttacaggccgaggatgtggctgtctactattgtcagcagttgtacacctaccccttcacattcggcggaggcaccaaggttgagatcaag

SEQ ID NO: 33 - DNA, 825 bp, synthetic construct
  source 1..825: mol_type = other DNA
ggtacccccgggcccatggctcttcctgtgacagctcttctgctgcccctggccctgcttctgcatgctgctagacctcagcttcagctccaagagagcggacctggcttagtgaagcccagcgaaaccctgtccctcacctgcaccgtttctggcggaagcatcagcagctccagctattactggggatggatcaggcagccccctggcaagggtttagaatggatcggctcgatatattactccggcagcacctactataaccccagcttgaagagccgggttaccatttctgtggacacatcaaagaaccagttcagcctgaagctgagctctgtgactgccgccgacacagctgtgtactactgtgccagagagacagactactccagcggcatgggctacggcatggatgtgtggggacaaggaaccaccgttactgtgagcagcggttccaccagcggctcaggcaagcctggctcaggagaaggaagcaccaagggggatatacagatgacacagagcccctccagcctgtccgccagcgttggcgatcgtgtaacgatcacctgccgggcctctcagagcatcaactcctacctcaattggtatcaacagaagccaggcaaggcccccaaattactcatctacgccgccagcagcttacagagcggggttccctctagattctccggctccggttctggaacagatttcaccctcactatctccagcttgcagcccgaggatttcgccacttattactgtcagcagagcctggccgaccccttcacattcggcggaggcacaaaggttgagatcaag

SEQ ID NO: 34 - DNA, 828 bp, synthetic construct
  source 1..828: mol_type = other DNA
ggtacccccgggcccatggctcttcctgtgacagctcttctgctgcccctggccctgcttctgcatgctgctagacctcaggttcagcttgtgcagagcggagctgaagttaagaagcctggcgcctctgtgaaggttagctgcaaggccagcggctacacattcaaggaatatggcatctcctgggttaggcaggctcccggccaaggcttagaatggatgggctggatctccgcctactccggccacacctactacgcccagaagcttcagggcagggttaccatgaccaccgacaccagcacctctaccgcctatatggagctgaggagcctgagatcggacgacacagctgtgtattactgcgccagaggcccccactacgacgactggtctggatttatcatctggttcgacccctgggggcagggcaccctggtcacagtttcttctggctccaccagcggaagcggcaagccaggctcaggcgaaggatctacaaaaggcgacatccaaatgacacagagccccagcagcttgagcgcctccgttggcgacagagttacaatcacctgcagggcctctcagagcatcagcagctatttgaattggtatcaacagaagccaggaaaggcccctaagctgctcatctacgctgccagctcgctccaatctggcgttcctagcagatttagcggctccggcagcggcacagactttactcttaccattagctccctgcagcccgaggacttcgctacctactattgccagcaaagctacagattccctcccacctttggccagggcacaaaggttgagatcaag

SEQ ID NO: 35 - DNA, 825 bp, synthetic construct
  source 1..825: mol_type = other DNA
ggtacccccgggcccatggctcttcctgtgacagctcttctgctgcccctggccctgcttctgcatgctgctagacctcaggttcagttacaagagagcggacctggcttagtgaagcccagcgaaaccctgtccctcacctgcaccgtttctggcggaagcatcagctctcccgaccattactggggatggatcaggcagccccctggcaagggtttggaatggatcggcagcatctacgccagcggcagcacattctacaacccctcgctcaaaagcagggttactatttctgtggacacaagcaaaaatcagttcagcctgaagctgagctctgtgactgccgccgacacagctgtgtactactgtgccagagagacagactactccagcgggatgggctacggcatggatgtgtggggacaaggaaccaccgttactgtgagcagcggctccacaagcggctcaggcaagcctggctcaggagaaggaagcaccaagggggacattcaaatgacccaaagcccctccagcctgtccgccagcgttggcgatagggttaccattacctgcagggccagccaaagcatcaactcctacctaaattggtatcaacagaagccaggcaaggcccccaaactactcatttacgccgccagcagcttacagagcggggttccctctagattctccggcagcggttctggaacagatttcactctcacaatatcttcgctgcagcccgaggatttcgctacctactattgccagcaatccctggccgaccccttcacattcggcggaggcacaaaggttgagatcaag

SEQ ID NO: 36 - DNA, 822 bp, synthetic construct
  source 1..822: mol_type = other DNA
ggtacccccgggcccatggctcttcctgtgacagctcttctgctgcccctggccctgcttctgcatgctgctagacctcagatcacattaaaagagagcggacctacactggtgaagcccacccaaacgcttaccctcacctgcacctttagcgggttcagcctggacacagagggcgttggcgttggatggatcaggcagcctcctggcaaagccctcgaatggcttgccctcatctacttcaacgaccagaagagatacagcccctccttaaaatctcggctcacaatcaccaaagacacaagcaaaaatcaggttgtgctcaccatgaccaacatggaccctgtggacaccgctgtgtactactgtgccagagacaccggctacagcagatggtactacgggatggacgtttggggccaaggcaccactgtgaccgtttccagcggctctacaagcggcagcgggaaacctggttctggagagggcagcacaaagggcgacatccagatgacgcaatcccccagctctgtgagcgcctctgtgggagacagagttacaatcacatgccgggcctcccagggcatcagctcttggctggcatggtatcaacagaagcctggcaaggctcccaagctgctcatctatgccgcctcctccttacaatctggagttccctccaggttcagcgggagcggctcaggaacagacttcacccttaccatctctagcctgcaacccgaggacttcgctacttattactgtcagcaggcctacgcctaccccatcacattcggcggaggaacaaaggttgagatcaag

SEQ ID NO: 37 - DNA, 852 bp, synthetic construct
  source 1..852: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcgagaaatactactggagctggatccggcagcctcccggcaaaggcttagaatggatcggcgagatttatcacagcgggctcaccaactacaaccccagcctgaaatctcgagttacaatctctgtggacacaagcaagaatcagttctccctgaagctgagcagcgttactgccgccgacacagctgtgtactattgcgccagagttagatacgacagcagcgacagctattactacagctatgactacggcatggatgtgtgggggcagggcaccaccgttactgtctcctctggatctaccagcggcagcggcaagcctggatctggcgaaggaagcacaaagggcgacattgtgctcacccagagccccgacagcctggctgtgtctttaggcgaaagggctaccatcaactgcaagagcagccagagcgttctgtacagcagcaacaacaagaactaccttgcttggtatcaacagaagcctggccagccccctaagctgctcatctactgggcctctagcagagagagcggggttcccgatcggtttagcggctccggctcaggaaccgatttcaccctcactatctccagcctccaggccgaggatgtggctgtctactattgtcagcagagctatagcttcccctggacattcggcggaggcaccaaggttgagatcaag

SEQ ID NO: 38 - DNA, 852 bp, synthetic construct
  source 1..852: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagccgctatgtgtggagctggatccggcagcctcctggcaaaggccttgaatggatcggagagatagacagcagcggcaagaccaactacaaccccagcctgaaatcacgcgttacaatctctgtggacacaagcaagaatcagttctccctgaagctgagcagcgttactgccgccgacacagctgtgtactattgcgccagagttagatacgacagctccgacagctattactacagctatgactacggcatggatgtgtgggggcagggcaccaccgttacagttagctctggaagcaccagcggctccggcaagcctggatctggtgaaggaagcacaaagggcgacattgtgctcacccagagccccgacagcctggctgtgtctttaggcgaaagggctaccatcaactgcaagagcagccagagcgttctgtacagcagcaacaacaagaactaccttgcatggtatcaacagaagcctggccagcctcccaagctgctcatctactgggcctctagcagagagagcggggttcccgatcgctttagcggcagcggttctggcaccgatttcactcttacaatcagcagcttacaggccgaggatgtggctgtctactattgtcagcagagctatagcttcccctggacattcggcggaggcaccaaggttgagatcaag

SEQ ID NO: 39 - DNA, 852 bp, synthetic construct
  source 1..852: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagcggctacgcttggagctggattagacagcctcctggcaaaggactagaatggatcggagagatcgaccacagaggcttcaccaactacaaccccagcctgaaatccagagttacaatctctgtggacacaagcaagaatcagttctccctgaagctgagcagcgttactgccgccgacacagctgtgtactattgcgccagggttagatacgacagcagcgacagctattactacagctatgactacggcatggatgtgtgggggcagggcaccaccgttacggttagctctggatctaccagcggcagcggcaagcctggctcaggagaaggaagcacaaagggcgacattgtgctcacccagagccccgacagcctggccgtttctttaggcgaaagggctaccatcaactgcaagagcagccagagcgttctgtacagcagcaacaacaagaactaccttgcatggtatcaacagaagccaggccagcctcccaagctgctcatctactgggcctctagcagagagagcggggttcccgatagattttcgggatcaggctccggcaccgatttcactcttacgatcagcagcttacaggccgaggatgtggctgtctactattgtcagcagagctatagcttcccctggacattcggcggaggcaccaaggttgagatcaag

SEQ ID NO: 40 - DNA, 840 bp, synthetic construct
  source 1..840: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttccagaaatactactggagctggatccggcagcctcccggcaaaggcttagaatggatcggagagatagacaccagcggcttcaccaactacaaccccagcctgaaatctagggttacaatctctgtggacacaagcaagaatcagttctccctgaagctgagcagcgttactgccgccgacacagctgtgtactattgcgccagagttggcagatacagctacggctactacatcaccgccttcgacatttggggccaaggcaccactgtgaccgtttccagcggaagcactagcggcagcgggaaacctggttctggagagggctcaaccaagggcgacatcgtgatgacacagagccccgactctctggctgtgtccctgggagagagagccaccatcaactgcaagagcagccagagcgttctgtacagcagcaacaacaagaactacctggcatggtatcaacagaagcctggccagccccctaagctgctcatctactgggcttccaccagagaatcaggcgttccagacaggttctccggctcgggttcaggcacagacttcacccttaccatctcttccctgcaggccgaagatgtggccgtttactactgtcagcagcactacagcttccctttcacattcggcggaggcaccaaggttgagatcaag

SEQ ID NO: 41 - DNA, 1577 bp, synthetic construct
  source 1..1577: mol_type = other DNA
ggtacccccgggcccatggctcttcctgtgacagctcttctgctgcccctggccctgcttctgcatgctgctagacctcagcttcagctccaagagagcggacctggcttagtgaagcccagcgaaaccctgtccctcacctgcaccgtttctggcggaagcatcagcagctccagctattactggggatggatcaggcagccccctggcaagggtttagaatggatcggctcgatatattactccggcagcacctactataaccccagcttgaagagccgggttaccatttctgtggacacatcaaagaaccagttcagcctgaagctgagctctgtgactgccgccgacacagctgtgtactactgtgccagagagacagactactccagcggcatgggctacggcatggatgtgtggggacaaggaaccaccgttactgtgagcagcggttccaccagcggctcaggcaagcctggctcaggagaaggaagcaccaagggggatatacagatgacacagagcccctccagcctgtccgccagcgttggcgatcgtgtaacgatcacctgccgggcctctcagagcatcaactcctacctcaattggtatcaacagaagccaggcaaggcccccaaattactcatctacgccgccagcagcttacagagcggggttccctctagattctccggctccggttctggaacagatttcaccctcactatctccagcttgcagcccgaggatttcgccacttattactgtcagcagagcctggccgaccccttcacattcggcggaggcacaaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 42 - DNA, 1577 bp, synthetic construct
  source 1..1577: mol_type = other DNA
ggtacccccgggcccatggctcttcctgtgacagctcttctgctgcccctggccctgcttctgcatgctgctagacctcaggttcagttacaagagagcggacctggcttagtgaagcccagcgaaaccctgtccctcacctgcaccgtttctggcggaagcatcagctctcccgaccattactggggatggatcaggcagccccctggcaagggtttggaatggatcggcagcatctacgccagcggcagcacattctacaacccctcgctcaaaagcagggttactatttctgtggacacaagcaaaaatcagttcagcctgaagctgagctctgtgactgccgccgacacagctgtgtactactgtgccagagagacagactactccagcgggatgggctacggcatggatgtgtggggacaaggaaccaccgttactgtgagcagcggctccacaagcggctcaggcaagcctggctcaggagaaggaagcaccaagggggacattcaaatgacccaaagcccctccagcctgtccgccagcgttggcgatagggttaccattacctgcagggccagccaaagcatcaactcctacctaaattggtatcaacagaagccaggcaaggcccccaaactactcatttacgccgccagcagcttacagagcggggttccctctagattctccggcagcggttctggaacagatttcactctcacaatatcttcgctgcagcccgaggatttcgctacctactattgccagcaatccctggccgaccccttcacattcggcggaggcacaaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 43 - DNA, 1574 bp, synthetic construct
  source 1..1574: mol_type = other DNA
ggtacccccgggcccatggctcttcctgtgacagctcttctgctgcccctggccctgcttctgcatgctgctagacctcagatcacattaaaagagagcggacctacactggtgaagcccacccaaacgcttaccctcacctgcacctttagcgggttcagcctggacacagagggcgttggcgttggatggatcaggcagcctcctggcaaagccctcgaatggcttgccctcatctacttcaacgaccagaagagatacagcccctccttaaaatctcggctcacaatcaccaaagacacaagcaaaaatcaggttgtgctcaccatgaccaacatggaccctgtggacaccgctgtgtactactgtgccagagacaccggctacagcagatggtactacgggatggacgtttggggccaaggcaccactgtgaccgtttccagcggctctacaagcggcagcgggaaacctggttctggagagggcagcacaaagggcgacatccagatgacgcaatcccccagctctgtgagcgcctctgtgggagacagagttacaatcacatgccgggcctcccagggcatcagctcttggctggcatggtatcaacagaagcctggcaaggctcccaagctgctcatctatgccgcctcctccttacaatctggagttccctccaggttcagcgggagcggctcaggaacagacttcacccttaccatctctagcctgcaacccgaggacttcgctacttattactgtcagcaggcctacgcctaccccatcacattcggcggaggaacaaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 44 - DNA, 1592 bp, synthetic construct
  source 1..1592: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttccagaaatactactggagctggatccggcagcctcccggcaaaggcttagaatggatcggagagatagacaccagcggcttcaccaactacaaccccagcctgaaatctagggttacaatctctgtggacacaagcaagaatcagttctccctgaagctgagcagcgttactgccgccgacacagctgtgtactattgcgccagagttggcagatacagctacggctactacatcaccgccttcgacatttggggccaaggcaccactgtgaccgtttccagcggaagcactagcggcagcgggaaacctggttctggagagggctcaaccaagggcgacatcgtgatgacacagagccccgactctctggctgtgtccctgggagagagagccaccatcaactgcaagagcagccagagcgttctgtacagcagcaacaacaagaactacctggcatggtatcaacagaagcctggccagccccctaagctgctcatctactgggcttccaccagagaatcaggcgttccagacaggttctccggctcgggttcaggcacagacttcacccttaccatctcttccctgcaggccgaagatgtggccgtttactactgtcagcagcactacagcttccctttcacattcggcggaggcaccaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 45 - DNA, 1604 bp, synthetic construct
  source 1..1604: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcgagaaatactactggagctggatccggcagcctcccggcaaaggcttagaatggatcggcgagatttatcacagcgggctcaccaactacaaccccagcctgaaatctcgagttacaatctctgtggacacaagcaagaatcagttctccctgaagctgagcagcgttactgccgccgacacagctgtgtactattgcgccagagttagatacgacagcagcgacagctattactacagctatgactacggcatggatgtgtgggggcagggcaccaccgttactgtctcctctggatctaccagcggcagcggcaagcctggatctggcgaaggaagcacaaagggcgacattgtgctcacccagagccccgacagcctggctgtgtctttaggcgaaagggctaccatcaactgcaagagcagccagagcgttctgtacagcagcaacaacaagaactaccttgcttggtatcaacagaagcctggccagccccctaagctgctcatctactgggcctctagcagagagagcggggttcccgatcggtttagcggctccggctcaggaaccgatttcaccctcactatctccagcctccaggccgaggatgtggctgtctactattgtcagcagagctatagcttcccctggacattcggcggaggcaccaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 46 - DNA, 1604 bp, synthetic construct
  source 1..1604: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagccgctatgtgtggagctggatccggcagcctcctggcaaaggccttgaatggatcggagagatagacagcagcggcaagaccaactacaaccccagcctgaaatcacgcgttacaatctctgtggacacaagcaagaatcagttctccctgaagctgagcagcgttactgccgccgacacagctgtgtactattgcgccagagttagatacgacagctccgacagctattactacagctatgactacggcatggatgtgtgggggcagggcaccaccgttacagttagctctggaagcaccagcggctccggcaagcctggatctggtgaaggaagcacaaagggcgacattgtgctcacccagagccccgacagcctggctgtgtctttaggcgaaagggctaccatcaactgcaagagcagccagagcgttctgtacagcagcaacaacaagaactaccttgcatggtatcaacagaagcctggccagcctcccaagctgctcatctactgggcctctagcagagagagcggggttcccgatcgctttagcggcagcggttctggcaccgatttcactcttacaatcagcagcttacaggccgaggatgtggctgtctactattgtcagcagagctatagcttcccctggacattcggcggaggcaccaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 47 - DNA, 1604 bp, synthetic construct
  source 1..1604: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagcggctacgcttggagctggattagacagcctcctggcaaaggactagaatggatcggagagatcgaccacagaggcttcaccaactacaaccccagcctgaaatccagagttacaatctctgtggacacaagcaagaatcagttctccctgaagctgagcagcgttactgccgccgacacagctgtgtactattgcgccagggttagatacgacagcagcgacagctattactacagctatgactacggcatggatgtgtgggggcagggcaccaccgttacggttagctctggatctaccagcggcagcggcaagcctggctcaggagaaggaagcacaaagggcgacattgtgctcacccagagccccgacagcctggccgtttctttaggcgaaagggctaccatcaactgcaagagcagccagagcgttctgtacagcagcaacaacaagaactaccttgcatggtatcaacagaagccaggccagcctcccaagctgctcatctactgggcctctagcagagagagcggggttcccgatagattttcgggatcaggctccggcaccgatttcactcttacgatcagcagcttacaggccgaggatgtggctgtctactattgtcagcagagctatagcttcccctggacattcggcggaggcaccaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 48 - DNA, 1565 bp, synthetic construct
  source 1..1565: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggctattactggagctggatccggcagcctcctggaaaaggattagaatggatcggcgagatagaccacagcgggagcacaaactacaaccccagcctgaaatcgcgggttacaatctctgtggacacaagcaagaatcagttctccctgaagctgagcagcgttactgccgccgacacagctgtgtactattgcgccagaggcggaggctcctggtacagcaactggttcgatccttggggccaaggcaccatggtgaccgtttccagcggctctacaagcggcagcgggaaacctggttctggagagggcagcacaaagggcgacatccagatgacacagagccccagcacccttagcgcctctgtgggagatagggttaccattacctgcagggcttcccagagcatcagcagctggctggcatggtatcaacagaagcctggcaaggctcccaagctgctcatctatgacgcctccagcctggaaagcggggttccctccagatttagcggctcaggctccggaacagagttcacccttaccatctctagcctgcaacccgacgacttcgctacttattactgtcaacaagacagaagcttgccccccacattcggcggagggaccaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 49 - DNA, 1580 bp, synthetic construct
  source 1..1580: mol_type = other DNA
ggtacccccgggcccatggctcttcctgtgacagctcttctgctgcccctggccctgcttctgcatgctgctagacctcaggttcagcttgtgcagagcggagctgaagttaagaagcctggcgcctctgtgaaggttagctgcaaggccagcggctacacattcaaggaatatggcatctcctgggttaggcaggctcccggccaaggcttagaatggatgggctggatctccgcctactccggccacacctactacgcccagaagcttcagggcagggttaccatgaccaccgacaccagcacctctaccgcctatatggagctgaggagcctgagatcggacgacacagctgtgtattactgcgccagaggcccccactacgacgactggtctggatttatcatctggttcgacccctgggggcagggcaccctggtcacagtttcttctggctccaccagcggaagcggcaagccaggctcaggcgaaggatctacaaaaggcgacatccaaatgacacagagccccagcagcttgagcgcctccgttggcgacagagttacaatcacctgcagggcctctcagagcatcagcagctatttgaattggtatcaacagaagccaggaaaggcccctaagctgctcatctacgctgccagctcgctccaatctggcgttcctagcagatttagcggctccggcagcggcacagactttactcttaccattagctccctgcagcccgaggacttcgctacctactattgccagcaaagctacagattccctcccacctttggccagggcacaaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 50 - DNA, 1586 bp, synthetic construct
  source 1..1586: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggcatccactggaactggatccggcagcctcctggcaaaggccttgaatggatcggcgatatcgacaccagcggctccaccaactacaaccccagcctgaaatcgagggttacaatctctgtggacacaagcaagaatcagttctccctgaagctgagcagcgttactgccgccgacacagctgtgtactattgcgccagactgggccaggaaagcgctacctaccttggcatggatgtgtgggggcagggcaccaccgttactgttagctctggctcaacaagcggcagcggcaagcctggctcaggagaaggaagcacaaagggcgacattgtaatgactcagagccccgacagcctggccgttagcttaggcgaaagggctacaatcaattgcaagagcagccagagcgttctgtacagcagcaacaacaagaactacctcgcatggtatcaacagaagccaggccagcctcccaagctgctcatctactgggcttccaccagagagagcggggttcccgatagattctccggctccggttctggaacagatttcacgctcacaatcagcagcttacaggccgaggatgtggctgtctactattgtcagcagttgtacacctaccccttcacattcggcggaggcaccaaggttgagatcaaggcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaacagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 51 - DNA, 1499 bp, synthetic construct
  source 1..1499: mol_type = other DNA
ggtacccccgggcccatggctcttcctgtgacagctcttctgctgcccctggccctgcttctgcatgctgctagacctcagcttcagctccaagagagcggacctggcttagtgaagcccagcgaaaccctgtccctcacctgcaccgtttctggcggaagcatcagcagctccagctattactggggatggatcaggcagccccctggcaagggtttagaatggatcggctcgatatattactccggcagcacctactataaccccagcttgaagagccgggttaccatttctgtggacacatcaaagaaccagttcagcctgaagctgagctctgtgactgccgccgacacagctgtgtactactgtgccagagagacagactactccagcggcatgggctacggcatggatgtgtggggacaaggaaccaccgttactgtgagcagcggttccaccagcggctcaggcaagcctggctcaggagaaggaagcaccaagggggatatacagatgacacagagcccctccagcctgtccgccagcgttggcgatcgtgtaacgatcacctgccgggcctctcagagcatcaactcctacctcaattggtatcaacagaagccaggcaaggcccccaaattactcatctacgccgccagcagcttacagagcggggttccctctagattctccggctccggttctggaacagatttcaccctcactatctccagcttgcagcccgaggatttcgccacttattactgtcagcagagcctggccgaccccttcacattcggcggaggcacaaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 52 - DNA, 1499 bp, synthetic construct
  source 1..1499: mol_type = other DNA
ggtacccccgggcccatggctcttcctgtgacagctcttctgctgcccctggccctgcttctgcatgctgctagacctcaggttcagttacaagagagcggacctggcttagtgaagcccagcgaaaccctgtccctcacctgcaccgtttctggcggaagcatcagctctcccgaccattactggggatggatcaggcagccccctggcaagggtttggaatggatcggcagcatctacgccagcggcagcacattctacaacccctcgctcaaaagcagggttactatttctgtggacacaagcaaaaatcagttcagcctgaagctgagctctgtgactgccgccgacacagctgtgtactactgtgccagagagacagactactccagcgggatgggctacggcatggatgtgtggggacaaggaaccaccgttactgtgagcagcggctccacaagcggctcaggcaagcctggctcaggagaaggaagcaccaagggggacattcaaatgacccaaagcccctccagcctgtccgccagcgttggcgatagggttaccattacctgcagggccagccaaagcatcaactcctacctaaattggtatcaacagaagccaggcaaggcccccaaactactcatttacgccgccagcagcttacagagcggggttccctctagattctccggcagcggttctggaacagatttcactctcacaatatcttcgctgcagcccgaggatttcgctacctactattgccagcaatccctggccgaccccttcacattcggcggaggcacaaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 53 - DNA, 1496 bp, synthetic construct
  source 1..1496: mol_type = other DNA
ggtacccccgggcccatggctcttcctgtgacagctcttctgctgcccctggccctgcttctgcatgctgctagacctcagatcacattaaaagagagcggacctacactggtgaagcccacccaaacgcttaccctcacctgcacctttagcgggttcagcctggacacagagggcgttggcgttggatggatcaggcagcctcctggcaaagccctcgaatggcttgccctcatctacttcaacgaccagaagagatacagcccctccttaaaatctcggctcacaatcaccaaagacacaagcaaaaatcaggttgtgctcaccatgaccaacatggaccctgtggacaccgctgtgtactactgtgccagagacaccggctacagcagatggtactacgggatggacgtttggggccaaggcaccactgtgaccgtttccagcggctctacaagcggcagcgggaaacctggttctggagagggcagcacaaagggcgacatccagatgacgcaatcccccagctctgtgagcgcctctgtgggagacagagttacaatcacatgccgggcctcccagggcatcagctcttggctggcatggtatcaacagaagcctggcaaggctcccaagctgctcatctatgccgcctcctccttacaatctggagttccctccaggttcagcgggagcggctcaggaacagacttcacccttaccatctctagcctgcaacccgaggacttcgctacttattactgtcagcaggcctacgcctaccccatcacattcggcggaggaacaaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 54 - DNA, 1514 bp, synthetic construct
  source 1..1514: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttccagaaatactactggagctggatccggcagcctcccggcaaaggcttagaatggatcggagagatagacaccagcggcttcaccaactacaaccccagcctgaaatctagggttacaatctctgtggacacaagcaagaatcagttctccctgaagctgagcagcgttactgccgccgacacagctgtgtactattgcgccagagttggcagatacagctacggctactacatcaccgccttcgacatttggggccaaggcaccactgtgaccgtttccagcggaagcactagcggcagcgggaaacctggttctggagagggctcaaccaagggcgacatcgtgatgacacagagccccgactctctggctgtgtccctgggagagagagccaccatcaactgcaagagcagccagagcgttctgtacagcagcaacaacaagaactacctggcatggtatcaacagaagcctggccagccccctaagctgctcatctactgggcttccaccagagaatcaggcgttccagacaggttctccggctcgggttcaggcacagacttcacccttaccatctcttccctgcaggccgaagatgtggccgtttactactgtcagcagcactacagcttccctttcacattcggcggaggcaccaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 55 - DNA, 1526 bp, synthetic construct
  source 1..1526: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcgagaaatactactggagctggatccggcagcctcccggcaaaggcttagaatggatcggcgagatttatcacagcgggctcaccaactacaaccccagcctgaaatctcgagttacaatctctgtggacacaagcaagaatcagttctccctgaagctgagcagcgttactgccgccgacacagctgtgtactattgcgccagagttagatacgacagcagcgacagctattactacagctatgactacggcatggatgtgtgggggcagggcaccaccgttactgtctcctctggatctaccagcggcagcggcaagcctggatctggcgaaggaagcacaaagggcgacattgtgctcacccagagccccgacagcctggctgtgtctttaggcgaaagggctaccatcaactgcaagagcagccagagcgttctgtacagcagcaacaacaagaactaccttgcttggtatcaacagaagcctggccagccccctaagctgctcatctactgggcctctagcagagagagcggggttcccgatcggtttagcggctccggctcaggaaccgatttcaccctcactatctccagcctccaggccgaggatgtggctgtctactattgtcagcagagctatagcttcccctggacattcggcggaggcaccaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 56 - DNA, 1526 bp, synthetic construct
  source 1..1526: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagccgctatgtgtggagctggatccggcagcctcctggcaaaggccttgaatggatcggagagatagacagcagcggcaagaccaactacaaccccagcctgaaatcacgcgttacaatctctgtggacacaagcaagaatcagttctccctgaagctgagcagcgttactgccgccgacacagctgtgtactattgcgccagagttagatacgacagctccgacagctattactacagctatgactacggcatggatgtgtgggggcagggcaccaccgttacagttagctctggaagcaccagcggctccggcaagcctggatctggtgaaggaagcacaaagggcgacattgtgctcacccagagccccgacagcctggctgtgtctttaggcgaaagggctaccatcaactgcaagagcagccagagcgttctgtacagcagcaacaacaagaactaccttgcatggtatcaacagaagcctggccagcctcccaagctgctcatctactgggcctctagcagagagagcggggttcccgatcgctttagcggcagcggttctggcaccgatttcactcttacaatcagcagcttacaggccgaggatgtggctgtctactattgtcagcagagctatagcttcccctggacattcggcggaggcaccaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 57 - DNA, 1526 bp, synthetic construct
  source 1..1526: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagcggctacgcttggagctggattagacagcctcctggcaaaggactagaatggatcggagagatcgaccacagaggcttcaccaactacaaccccagcctgaaatccagagttacaatctctgtggacacaagcaagaatcagttctccctgaagctgagcagcgttactgccgccgacacagctgtgtactattgcgccagggttagatacgacagcagcgacagctattactacagctatgactacggcatggatgtgtgggggcagggcaccaccgttacggttagctctggatctaccagcggcagcggcaagcctggctcaggagaaggaagcacaaagggcgacattgtgctcacccagagccccgacagcctggccgtttctttaggcgaaagggctaccatcaactgcaagagcagccagagcgttctgtacagcagcaacaacaagaactaccttgcatggtatcaacagaagccaggccagcctcccaagctgctcatctactgggcctctagcagagagagcggggttcccgatagattttcgggatcaggctccggcaccgatttcactcttacgatcagcagcttacaggccgaggatgtggctgtctactattgtcagcagagctatagcttcccctggacattcggcggaggcaccaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 58 - DNA, 1487 bp, synthetic construct
  source 1..1487: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggctattactggagctggatccggcagcctcctggaaaaggattagaatggatcggcgagatagaccacagcgggagcacaaactacaaccccagcctgaaatcgcgggttacaatctctgtggacacaagcaagaatcagttctccctgaagctgagcagcgttactgccgccgacacagctgtgtactattgcgccagaggcggaggctcctggtacagcaactggttcgatccttggggccaaggcaccatggtgaccgtttccagcggctctacaagcggcagcgggaaacctggttctggagagggcagcacaaagggcgacatccagatgacacagagccccagcacccttagcgcctctgtgggagatagggttaccattacctgcagggcttcccagagcatcagcagctggctggcatggtatcaacagaagcctggcaaggctcccaagctgctcatctatgacgcctccagcctggaaagcggggttccctccagatttagcggctcaggctccggaacagagttcacccttaccatctctagcctgcaacccgacgacttcgctacttattactgtcaacaagacagaagcttgccccccacattcggcggagggaccaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 59 - DNA, 1502 bp, synthetic construct
  source 1..1502: mol_type = other DNA
ggtacccccgggcccatggctcttcctgtgacagctcttctgctgcccctggccctgcttctgcatgctgctagacctcaggttcagcttgtgcagagcggagctgaagttaagaagcctggcgcctctgtgaaggttagctgcaaggccagcggctacacattcaaggaatatggcatctcctgggttaggcaggctcccggccaaggcttagaatggatgggctggatctccgcctactccggccacacctactacgcccagaagcttcagggcagggttaccatgaccaccgacaccagcacctctaccgcctatatggagctgaggagcctgagatcggacgacacagctgtgtattactgcgccagaggcccccactacgacgactggtctggatttatcatctggttcgacccctgggggcagggcaccctggtcacagtttcttctggctccaccagcggaagcggcaagccaggctcaggcgaaggatctacaaaaggcgacatccaaatgacacagagccccagcagcttgagcgcctccgttggcgacagagttacaatcacctgcagggcctctcagagcatcagcagctatttgaattggtatcaacagaagccaggaaaggcccctaagctgctcatctacgctgccagctcgctccaatctggcgttcctagcagatttagcggctccggcagcggcacagactttactcttaccattagctccctgcagcccgaggacttcgctacctactattgccagcaaagctacagattccctcccacctttggccagggcacaaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 60 - DNA, 1508 bp, synthetic construct
  source 1..1508: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggcatccactggaactggatccggcagcctcctggcaaaggccttgaatggatcggcgatatcgacaccagcggctccaccaactacaaccccagcctgaaatcgagggttacaatctctgtggacacaagcaagaatcagttctccctgaagctgagcagcgttactgccgccgacacagctgtgtactattgcgccagactgggccaggaaagcgctacctaccttggcatggatgtgtgggggcagggcaccaccgttactgttagctctggctcaacaagcggcagcggcaagcctggctcaggagaaggaagcacaaagggcgacattgtaatgactcagagccccgacagcctggccgttagcttaggcgaaagggctacaatcaattgcaagagcagccagagcgttctgtacagcagcaacaacaagaactacctcgcatggtatcaacagaagccaggccagcctcccaagctgctcatctactgggcttccaccagagagagcggggttcccgatagattctccggctccggttctggaacagatttcacgctcacaatcagcagcttacaggccgaggatgtggctgtctactattgtcagcagttgtacacctaccccttcacattcggcggaggcaccaaggttgagatcaaggctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggttagattcagcgttgtgaagagaggccggaagaagctgctgtacatcttcaagcagcccttcatgagacctgtgcagaccacacaggaggaagacggctgcagctgtagattccccgaggaagaggagggcggctgtgagctgagagttaagttcagcaggagcgccgacgcccctgcctaccagcaaggacagaatcaactgtacaacgagctgaacctgggcagacgggaggaatacgatgtgctggacaagaggagaggcagagaccccgagatgggcggcaaacctagaagaaagaacccccaggagggcctgtataacgagctccagaaggacaagatggccgaggcctacagcgagatcggcatgaagggcgaaagaagaagaggcaagggccacgacggcctctaccagggcttaagcacagctacaaaggacacctacgacgccctgcacatgcaggccctgccccctagatgattaattaaatcgat

SEQ ID NO: 61 - DNA, 1577 bp, synthetic construct; identical to SEQ ID NO: 41
  source 1..1577: mol_type = other DNA

SEQ ID NO: 62 - DNA, 1577 bp, synthetic construct; identical to SEQ ID NO: 42
  source 1..1577: mol_type = other DNA

SEQ ID NO: 63 - DNA, 1574 bp, synthetic construct; identical to SEQ ID NO: 43
  source 1..1574: mol_type = other DNA

SEQ ID NO: 64 - DNA, 1592 bp, synthetic construct; identical to SEQ ID NO: 44
  source 1..1592: mol_type = other DNA

SEQ ID NO: 65 - protein, 458 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 467 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 494 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 458 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 467 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 492 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTADTAVYYCVSLVYCGGDCYSGFDYWGQGTLVTVSSAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 460 aa, synthetic construct
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 469 aa, synthetic construct
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 496 aa, synthetic construct
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 457 aa, synthetic construct
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 466 aa, synthetic construct
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 493 aa, synthetic construct
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - skipped

SEQ ID NO: 78 - protein, 453 aa, synthetic construct
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 462 aa, synthetic construct
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 489 aa, synthetic construct
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 453 aa, synthetic construct
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 462 aa, synthetic construct
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 489 aa, synthetic construct
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - DNA, 20 bp, synthetic construct
  source 1..20: mol_type = other DNA
cttcaagagcaacagtgctg

SEQ ID NO: 85 - DNA, 20 bp, synthetic construct
  source 1..20: mol_type = other DNA
ctctcagctggtacacggca

SEQ ID NO: 86 - DNA, 20 bp, synthetic construct
  source 1..20: mol_type = other DNA
tctctcagctggtacacggc

SEQ ID NO: 87 - DNA, 20 bp, synthetic construct
  source 1..20: mol_type = other DNA
gctggtacacggcagggtca

SEQ ID NO: 88 - DNA, 20 bp, synthetic construct
  source 1..20: mol_type = other DNA
ggccgagatgtctcgctccg

SEQ ID NO: 89 - DNA, 20 bp, synthetic construct
  source 1..20: mol_type = other DNA
cgcgagcacagctaaggcca

SEQ ID NO: 90 - DNA, 20 bp, synthetic construct
  source 1..20: mol_type = other DNA
gagtagcgcgagcacagcta

SEQ ID NO: 91 - DNA, 20 bp, synthetic construct
  source 1..20: mol_type = other DNA
aagtcaacttcaatgtcgga

SEQ ID NO: 92 - DNA, 20 bp, synthetic construct
  source 1..20: mol_type = other DNA
tcggagcctctgaagaaggg

SEQ ID NO: 93 - DNA, 20 bp, synthetic construct
  source 1..20: mol_type = other DNA
gccaagtacactgagcaatg

SEQ ID NO: 94 - DNA, 20 bp, synthetic construct
  source 1..20: mol_type = other DNA
caggggtggcatagacacca

SEQ ID NO: 95 - DNA, 20 bp, synthetic construct
  source 1..20: mol_type = other DNA
gccaagtacactgagcaatg

SEQ ID NO: 96 - protein, 21 aa, synthetic construct
MALPVTALLLPLALLLHAARP